(19) United States
(12) Patent Application Publication
(10) Pub. No.: US 2014/0334653 A1
(43) Pub. Date: Nov. 13, 2014

(54) COMBINATION SPEAKER AND LIGHT SOURCE RESPONSIVE TO STATE(S) OF AN ORGANISM BASED ON SENSOR DATA

(71) Applicants: Michael Edward Smith Luna, San Jose, CA (US); Derek Boyd Barrentine, Gilroy, CA (US); Scott Fullam, Palo Alto, CA (US); Sankalita Saha, Union City, CA (US); Patrick Alan Narron, Boulder Creek, CA (US); Jeremiah Robison, San Francisco, CA (US)

(72) Inventors: Michael Edward Smith Luna, San Jose, CA (US); Derek Boyd Barrentine, Gilroy, CA (US); Scott Fullam, Palo Alto, CA (US); Sankalita Saha, Union City, CA (US); Patrick Alan Narron, Boulder Creek, CA (US); Jeremiah Robison, San Francisco, CA (US)

(73) Assignee: AliphCom, San Francisco, CA (US)

(21) Appl. No.: 14/281,856

(22) Filed: May 19, 2014

Related U.S. Application Data
(63) Continuation-in-part of application No. 14/209,329, filed on Mar. 13, 2014.
Application No. 14/209,329 is a continuation-in-part of application No. 14/212,832, filed on Mar. 14, 2014.
Application No. 14/212,832 is a continuation-in-part of application No. 14/207,420, filed on Mar. 12, 2014.
(60) Provisional application No. 61/825,509, filed on May 20, 2013.
(60) Provisional application No. 61/825,509, filed on May 20, 2013.
(60) Provisional application No. 61/786,179, filed on Mar. 14, 2013.
(60) Provisional application No. 61/786,473, filed on Mar. 15, 2013.
(60) Provisional application No. 61/786,473, filed on Mar. 15, 2013.
(60) Provisional application No. 61/786,473, filed on Mar. 15, 2013.

(51) Int. Cl. G05B 15/02; H04R 3/00; A61M 21/02; H04R 1/02

(52) U.S. Cl. CPC G05B 15/02; H04R 1/028; H04R 3/00; A61M 21/02; A61M 2021/0016
USPC 381/332; 600/27; 600/28

(57) ABSTRACT

Techniques associated with a combination speaker and light source (“speaker-light device”) responsive to states of an organism based on sensor data are described, including generating chemical sensor data in response to one or more chemicals sensed by one or more chemical sensors in the same or different speaker-light devices. A scent generator may be activated to counter an odor caused by a chemical detected by the chemical sensor(s). The speaker-light device may activate an air mover operative to circulate ambient air over the chemical sensor. The speaker-light device may take an appropriate action in response to detected chemicals that affect states of the organism. Some or all of the actions taken may be taken by other devices in communication with the speaker-light device(s). An action may include generating and presenting a path or route to be taken by a user to an area of safety or reduced chemical concentration.

### CROSS-REFERENCE TO RELATED APPLICATIONS

This application claims the benefit of U.S. Provisional Patent Application No. 61/825,509 (Attorney Docket No. ALI-274P), filed on May 20, 2013; this application is a continuation-in-part of U.S. patent application Ser. No. 14/209,329 (Attorney Docket No. ALI-421), filed on Mar. 13, 2014, which claims the benefit of U.S. Provisional Patent Application No. 61/825,509 (Attorney Docket No. ALI-274P), filed on May 20, 2013, U.S. Provisional Patent Application No. 61/786,179 (Attorney Docket No. ALI-270P), filed on Mar. 14, 2013, and U.S. Provisional Patent Application No. 61/786,473 (Attorney Docket No. ALI-271P), filed on Mar. 15, 2013; this application is a continuation-in-part of U.S. patent application Ser. No. 14/212,832 (Attorney Docket No. ALI-418), filed on Mar. 14, 2014, which claims the benefit U.S. Provisional Patent Application No. 61/786,473 (Attorney Docket No. ALI-271P), filed on Mar. 15, 2013; and this application is a continuation-in-part of U.S. patent application Ser. No. 14/207,420 (Attorney Docket No. ALI-271), filed on Mar. 12, 2014, which claims the benefit U.S. Provisional Patent Application No. 61/786,473 (Attorney Docket No. ALI-271P), filed on Mar. 15, 2013, all of which are incorporated by reference herein in their entirety for all purposes.

### FIELD

The present invention relates generally to electrical and electronic hardware, electromechanical and computing devices. More specifically, techniques related to a combination speaker and light source responsive to states of an organism based on sensor data are described.

### BACKGROUND

Conventional devices for lighting typically do not provide audio playback capabilities, and conventional devices for audio playback (i.e., speakers) typically do not provide light. Although there are conventional speakers equipped with light features for decoration or as part of a user interface, such conventional speakers are typically not configured to provide ambient lighting or the light an environment. Also, conventional speakers typically are not configured to be installed into or powered using a light socket.

Conventional devices for lighting and playing audio also typically lack capabilities for responding automatically to a person's state and environment, particularly in a contextually-meaningful manner.

Thus, what is needed is a solution for a combination speaker and light source responsive to states of an organism based on sensor data without the limitations of conventional techniques.

### BRIEF DESCRIPTION OF THE DRAWINGS

Various embodiments or examples (“examples”) are disclosed in the following detailed description and the accompanying drawings:

FIG. 1A illustrates an exemplary array of electrodes and a physiological information generator disposed in a wearable data-capable band, according to some embodiments;

FIGS. 1B to 1D illustrate examples of electrode arrays, according to some embodiments;

FIG. 2 is a functional diagram depicting a physiological information generator implemented in a wearable device, according to some embodiments;

FIGS. 3A to 3C are cross-sectional views depicting arrays of electrodes including subsets of electrodes adjacent an arm of a wearer, according to some embodiments;

FIG. 4 depicts a portion of an array of electrodes disposed within a housing material of a wearable device, according to some embodiments;

FIG. 5 depicts an example of a physiological information generator, according to some embodiments;

FIG. 6 is an example flow diagram for selecting a sensor, according to some embodiments;

FIG. 7 is an example flow diagram for determining physiological characteristics using a wearable device with arrayed electrodes, according to some embodiments;

FIG. 8 illustrates an exemplary computing platform disposed in a wearable device in accordance with various embodiments

FIG. 9 depicts the physiological signal extractor, according to some embodiments;

FIG. 10 is a flowchart for extracting a physiological signal, according to some embodiments;

FIG. 11 is a block diagram depicting an example of a physiological signal extractor, according to some embodiments;

FIG. 12 depicts an example of an offset generator, according to some embodiments;

FIG. 13 is a flowchart depicting example of a flow for decomposing a sensor signal to form separate signals, according to some embodiments;

FIGS. 14A to 14D depict various signals used for physiological characteristic signal extraction, according to various embodiments;

FIG. 15 depicts recovered signals, according to some embodiments;

FIG. 16 depicts an extracted physiological signal, according to various embodiments;

FIG. 17 illustrates an exemplary computing platform disposed in a wearable device in accordance with various embodiments;

FIG. 18 is a diagram depicting a physiological state determinator configured to receive sensor data originating, for example, at a distal portion of a limb, according to some embodiments;

FIG. 19 depicts a sleep manager, according to some embodiments;

FIG. 20A depicts a wearable device including a skin surface microphone (“SSM”), according to some embodiments;

FIG. 20B depicts an example of data arrangements for physiological characteristics and parametric values that can identify a sleep state, according to some embodiments;

FIG. 21 depicts an anomalous state manager, according to some embodiments;

FIG. 22 depicts an affective state manager configured to receive sensor data derived from bioimpedance signals, according to some embodiments;

FIG. 23 illustrates an exemplary computing platform disposed in a wearable device in accordance with various embodiments;

FIGS. 24A to 24B illustrate exemplary combination speaker and light source devices powered using a light socket;

FIG. 25 illustrates an exemplary system for manipulating a combination speaker and light source according to a physiological state determined using sensor data;

FIG. 26 illustrates an exemplary architecture for a combination speaker and light source device;

FIGS. 27A to 27B illustrate side-views of exemplary combination speaker and light source devices;

FIG. 27C illustrates a top-view of an exemplary combination speaker and light source device;

FIG. 28 illustrates an exemplary computing platform disposed in or associated with a combination speaker and light source device;

FIGS. 29A-29B illustrate exemplary flows for a combination speaker and light source device;

FIG. 30 illustrates an exemplary system for controlling a combination speaker and light source device according to a physiological state;

FIG. 31 illustrates an exemplary flow for controlling a combination speaker and light source device according to a physiological state;

FIG. 32 depicts an exemplary system for controlling a combination speaker and light source device according to a physiological state and/or chemicals sensed by a chemical sensor which may also be referred to as an environmental sensor;

FIG. 33 depicts an exemplary architecture for a combination speaker and light source device including a chemical sensor;

FIG. 34 depicts a top-view of an exemplary combination speaker and light source device including a chemical sensor;

FIG. 35 depicts a cross-sectional view of an exemplary combination speaker and light source device including a chemical sensor;

FIG. 36 depicts a cross-sectional view of an exemplary combination speaker and light source device including a chemical sensor and an air mover;

FIG. 37 depicts a cross-sectional view of examples of a combination speaker and light source device including a chemical sensor coupled with an external air mover and a combination speaker and light source device including a chemical sensor positioned in proximity of an external air mover;

FIG. 38 depicts top plan views of a structure that includes a plurality of combination speaker and light source devices positioned in a plurality of rooms on different floors of the structure;

FIG. 39 depicts examples of routes generated by one or more combination speaker and light source devices; and

FIG. 40 illustrates an exemplary flow for controlling a combination speaker and light source device according to detection of one or more chemicals by one or more chemical sensors.

Although the above-described drawings depict various examples of the invention, the invention is not limited by the depicted examples. It is to be understood that, in the drawings, like reference numerals designate like structural elements. Also, it is understood that the drawings are not necessarily to scale.

### DETAILED DESCRIPTION

Various embodiments or examples may be implemented in numerous ways, including as a system, a process, an apparatus, a device, and a method associated with a wearable device structure with enhanced detection by motion sensor. In some embodiments, motion may be detected using an accelerometer that responds to an applied force and produces an output signal representative of the acceleration (and hence in some cases a velocity or displacement) produced by the force. Embodiments may be used to couple or secure a wearable device onto a body part. Techniques described are directed to systems, apparatuses, devices, and methods for using accelerometers, or other devices capable of detecting motion, to detect the motion of an element or part of an overall system. In some examples, the described techniques may be used to accurately and reliably detect the motion of a part of the human body or an element of another complex system. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

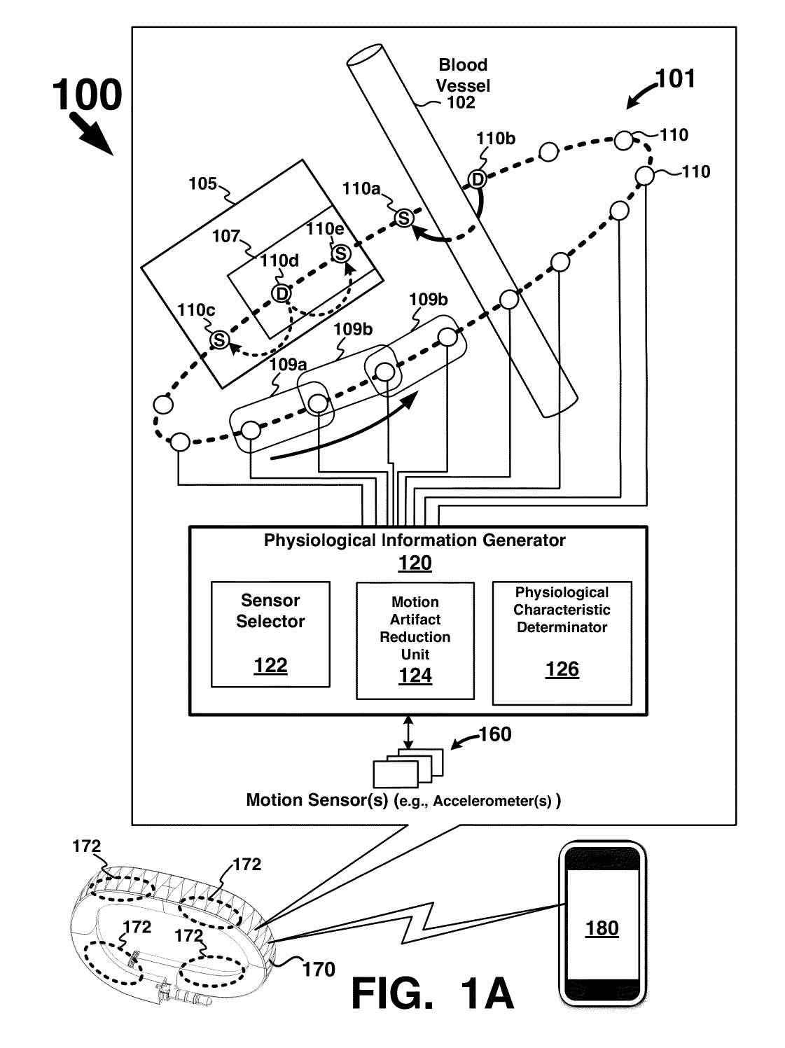

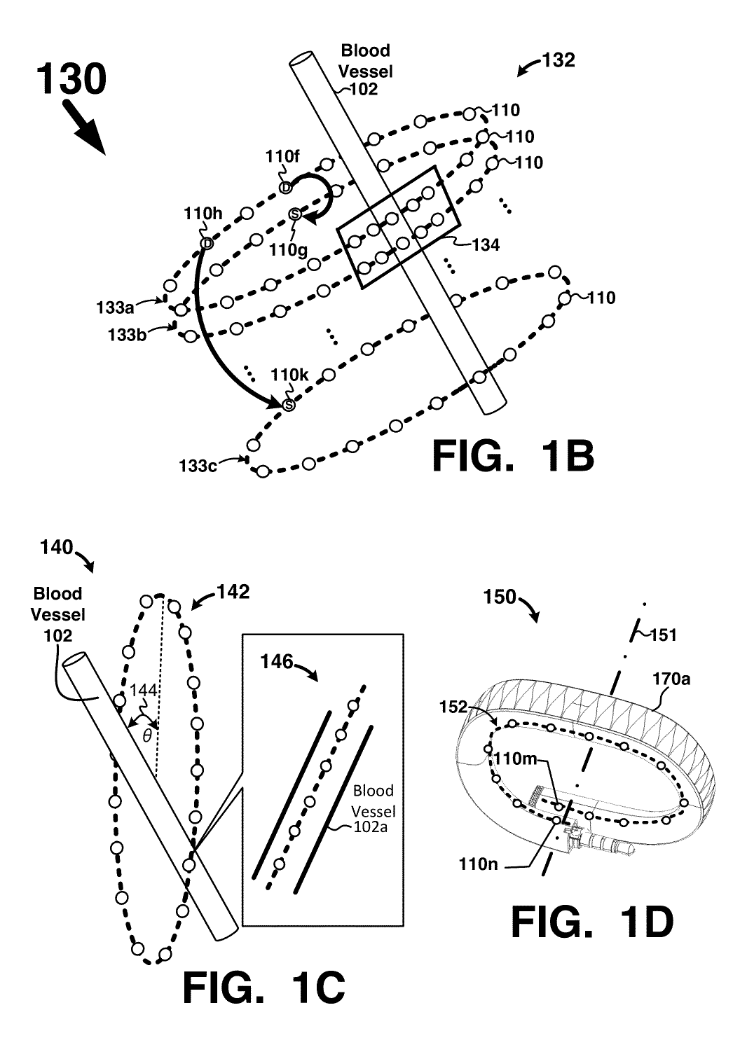

FIG. 1A illustrates an exemplary array of electrodes and a physiological information generator disposed in a wearable data-capable band, according to some embodiments. Diagram 100 depicts an array 100 of electrodes 110 coupled to a physiological information generator 120 that is configured to generate data representing one or more physiological characteristics associated with a user that is wearing or carrying array 101. Also shown are motion sensors 160, which, for example, can include accelerometers. Motion sensors 160 are not limited to accelerometers. Examples of motion sensors 160 can also include gyroscopic sensors, optical motion sensors (e.g., laser or LED motion detectors, such as used in optical mice), magnet-based motion sensors (e.g., detecting magnetic fields, or changes thereof, to detect motion), electromagnetic-based sensors, etc., as well as any sensor configured to detect or determine motion, such as motion sensors based on physiological characteristics (e.g., using electromyography (“EMG”) to determine existence and/or amounts of motion based on electrical signals generated by muscle cells), and the like. Electrodes 110 can include any suitable structure for transferring signals and picking up signals, regardless of whether the signals are electrical, magnetic, optical, pressure-based, physical, acoustic, etc., according to various embodiments. According to some embodiments, electrodes 110 of array 101 are configured to couple capacitively to a target location. In some embodiments, array 101 and physiological information generator 120 are disposed in a wearable device, such as a wearable data-capable band 170, which may include a housing that encapsulates, or substantially encapsulates, array 101 of electrodes 110. In operations, physiological information generator 120 can determine the bioelectric impedance (“bioimpedance”) of one or more types of tissues of a wearer to identify, measure, and monitor physiological characteristics. For example, a drive signal having a known amplitude and frequency can be applied to a user, from which a sink signal is received as bioimpedance signal. The bioimpedance signal is a measured signal that includes real and complex components. Examples of real components include extra-cellular and intra-cellular spaces of tissue, among other things, and examples of complex components include cellular membrane capacitance, among other things. Further, the measured bioimpedance signal can include real and/or complex components associated with arterial structures (e.g., arterial cells, etc.) and the presence (or absence) of blood pulsing through an arterial structure. In some examples, a heart rate signal, or other physiological signals, can be determined (i.e., recovered) from the measured bioimpedance signal by, for example, comparing the measured bioimpedance signal against the waveform of the drive signal to determine a phase delay (or shift) of the measured complex components.

Physiological information generator 120 is shown to include a sensor selector 122, a motion artifact reduction unit 124, and a physiological characteristic determinator 126. Sensor selector 122 is configured to select a subset of electrodes, and is further configured to use the selected subset of electrodes to acquire physiological characteristics, according to some embodiments. Examples of a subset of electrodes include subset 107, which is composed of electrodes 110d and 110e, and subset 105, which is composed of electrodes 110c, 110d and 110e. More or fewer electrodes can be used. Sensor selector 122 is configured to determine which one or more subsets of electrodes 110 (out of a number of subsets of electrodes 110) are adjacent to a target location. As used herein, the term “target location” can, for example, refer to a region in space from which a physiological characteristic can be determined. A target region can be adjacent to a source of the physiological characteristic, such as blood vessel 102, with which an impedance signal can be captured and analyzed to identify one or more physiological characteristics. The target region can reside in two-dimensional space, such as an area on the skin of a user adjacent to the source of the physiological characteristic, or in three-dimensional space, such as a volume that includes the source of the physiological characteristic. Sensor selector 122 operates to either drive a first signal via a selected subset to a target location, or receive a second signal from the target location, or both. The second signal includes data representing one or more physiological characteristics. For example, sensor selector 122 can configure electrode (“D”) 110b to operate as a drive electrode that drives a signal (e.g., an AC signal) into the target location, such as into the skin of a user, and can configure electrode (“S”) 110a to operate as a sink electrode (i.e., a receiver electrode) to receive a second signal from the target location, such as from the skin of the user. In this configuration, sensor selector 112 can drive a current signal via electrode (“D”) 110b into a target location to cause a current to pass through the target location to another electrode (“S”) 110a. In various examples, the target location can be adjacent to or can include blood vessel 102. Examples of blood vessel 102 include a radial artery, an ulnar artery, or any other blood vessel. Array 101 is not limited to being disposed adjacent blood vessel 102 in an arm, but can be disposed on any portion of a user's person (e.g., on an ankle, ear lobe, around a finger or on a fingertip, etc.). Note that each electrode 110 can be configured as either a driver or a sink electrode. Thus, electrode 110b is not limited to being a driver electrode and can be configured as a sink electrode in some implementations. As used herein, the term “sensor” can refer, for example, to a combination of one or more driver electrodes and one or more sink electrodes for determining one or more bioimpedance-related values and/or signals, according to some embodiments.

In some embodiments, sensor selector 122 can be configured to determine (periodically or aperiodically) whether the subset of electrodes 110a and 110b are optimal electrodes 110 for acquiring a sufficient representation of the one or more physiological characteristics from the second signal. To illustrate, consider that electrodes 110a and 110b may be displaced from the target location when, for instance, wearable device 170 is subject to a displacement in a plane substantially perpendicular to blood vessel 102. The displacement of electrodes 110a and 110b may increase the impedance (and/or reactance) of a current path between the electrodes 110a and 110b, or otherwise move those electrodes away from the target location far enough to degrade or attenuate the second signals retrieved therefrom. While electrodes 110a and 110b may be displaced from the target location, other electrodes are displaced to a position previously occupied by electrodes 110a and 110b (i.e., adjacent to the target location). For example, electrodes 110c and 110d may be displaced to a position adjacent to blood vessel 102. In this case, sensor selector 122 operates to determine an optimal subset of electrodes 110, such as electrodes 110c and 110d, to acquire the one or more physiological characteristics. Therefore, regardless of the displacement of wearable device 170 about blood vessel 102, sensor selector 122 can repeatedly determine an optimal subset of electrodes for extracting physiological characteristic information from adjacent a blood vessel. For example, sensor selector 122 can repeatedly test subsets in sequence (or in any other matter) to determine which one is disposed adjacent to a target location. For example, sensor selector 122 can select at least one of subset 109a, subset 109b, subset 109c, and other like subsets, as the subset from which to acquire physiological data.

According to some embodiments, array 101 of electrodes can be configured to acquire one or more physiological characteristics from multiple sources, such as multiple blood vessels. To illustrate, consider that, for example, blood vessel 102 is an ulnar artery adjacent electrodes 110a and 110b and a radial artery (not shown) is adjacent electrodes 110c and 110d. With multiple sources of physiological characteristic information being available, there are thus multiple target locations. Therefore, sensor selector 122 can select multiple subsets of electrodes 110, each of which is adjacent to one of a multiple number of target locations. Physiological information generator 120 then can use signal data from each of the multiple sources to confirm accuracy of data acquired, or to use one subset of electrodes (e.g., associated with a radial artery) when one or more other subsets of electrodes (e.g., associated with an ulnar artery) are unavailable.

Note that the second signal received into electrode 110a can be composed of a physiological-related signal component and a motion-related signal component, if array 101 is subject to motion. The motion-related component includes motion artifacts or noise induced into an electrode 110a. Motion artifact reduction unit 124 is configured to receive motion-related signals generated at one or more motion sensors 160, and is further configured to receive at least the motion-related signal component of the second signal. Motion artifact reduction unit 124 operates to eliminate the magnitude of the motion-related signal component, or to reduce the magnitude of the motion-related signal component relative to the magnitude of the physiological-related signal component, thereby yielding as an output the physiological-related signal component (or an approximation thereto). Thus, motion artifact reduction unit 124 can reduce the magnitude of the motion-related signal component (i.e., the motion artifact) by an amount associated with the motion-related signal generated by one or more accelerometers to yield the physiological-related signal component.

Physiological characteristic determinator 126 is configured to receive the physiological-related signal component of the second signal and is further configured to process (e.g., digitally) the signal data including one or more physiological characteristics to derive physiological signals, such as either a heart rate (“HR”) signal or a respiration signal, or both. For example, physiological characteristic determinator 126 is configured to amplify and/or filter the physiological-related component signals (e.g., at different frequency ranges) to extract certain physiological signals. According to various embodiments, a heart rate signal can include (or can be based on) a pulse wave. A pulse wave includes systolic components based on an initial pulse wave portion generated by a contracting heart, and diastolic components based on a reflected wave portion generated by the reflection of the initial pulse wave portion from other limbs. In some examples, an HR signal can include or otherwise relate to an electrocardiogram (“ECG”) signal. Physiological characteristic determinator 126 is further configured to calculate other physiological characteristics based on the acquired one or more physiological characteristics. Optionally, physiological characteristic determinator 126 can use other information to calculate or derive physiological characteristics. Examples of the other information include motion-related data, including the type of activity in which the user is engaged, such as running or sleep, location-related data, environmental-related data, such as temperature, atmospheric pressure, noise levels, etc., and any other type of sensor data, including stress-related levels and activity levels of the wearer.

In some cases, a motion sensor 160 can be disposed adjacent to the target location (not shown) to determine a physiological characteristic via motion data indicative of movement of blood vessel 102 through which blood pulses to identify a heart rate-related physiological characteristic. Motion data, therefore, can be used to supplement impedance determinations of to obtain the physiological characteristic. Further, one or more motion sensors 160 can also be used to determine the orientation of wearable device 170, and relative movement of the same to determine or predict a target location. By predicting a target location, sensor selector 122 can use the predicted target location to begin the selection of optimal subsets of electrodes 110 in a manner that reduces the time to identify a target location.

In view of the foregoing, the functions and/or structures of array 101 of electrodes and physiological information generator 120, as well as their components, can facilitate the acquisition and derivation of physiological characteristics in situ—during which a user is engaged in physical activity that imparts motion on a wearable device, thereby exposing the array of electrodes to motion-related artifacts. Physiological information generator 120 is configured to dampen or otherwise negate the motion-related artifacts from the signals received from the target location, thereby facilitating the provision of heart-related activity and respiration activity to the wearer of wearable device 170 in real-time (or near real-time). As such, the wearer of wearable device 170 need not be stationary or otherwise interrupt an activity in which the wearer is engaged to acquire health-related information. Also, array 101 of electrodes 110 and physiological information generator 120 are configured to accommodate displacement or movement of wearable device 170 about, or relative to, one or more target locations. For example, if the wearer intentionally rotates wearable device 170 about, for example, the wrist of the user, then initial subsets of electrodes 110 adjacent to the target locations (i.e., before the rotation) are moved further away from the target location. As another example, the motion of the wearer (e.g., impact forces experienced during running) may cause wearable device 170 to travel about the wrist. As such, physiological information generator 120 is configured to determine repeatedly whether to select other subsets of electrodes 110 as optimal subsets of electrodes 110 for acquiring physiological characteristics. For example, physiological information generator 120 can be configured to cycle through multiple combinations of driver electrodes and sink electrodes (e.g., subsets 109a, 109b, 109c, etc.) to determine optimal subsets of electrodes. In some embodiments, electrodes 110 in array 101 facilitate physiological data capture irrespective of the gender of the wearer. For example, electrodes 110 can be disposed in array 101 to accommodate data collection of a male or female were irrespective of gender-specific physiological dimensions. In at least one embodiment, data representing the gender of the wearer can be accessible to assist physiological information generator 120 in selecting the optimal subsets of electrodes 110. While electrodes 110 are depicted as being equally-spaced, array 101 is not so limited. In some embodiments, electrodes 110 can be clustered more densely along portions of array 101 at which blood vessels 102 are more likely to be adjacent. For example, electrodes 110 may be clustered more densely at approximate portions 172 of wearable device 170, whereby approximate portions 172 are more likely to be adjacent a radial or ulnar artery than other portions. While wearable device 170 is shown to have an elliptical-like shape, it is not limited to such a shape and can have any shape.

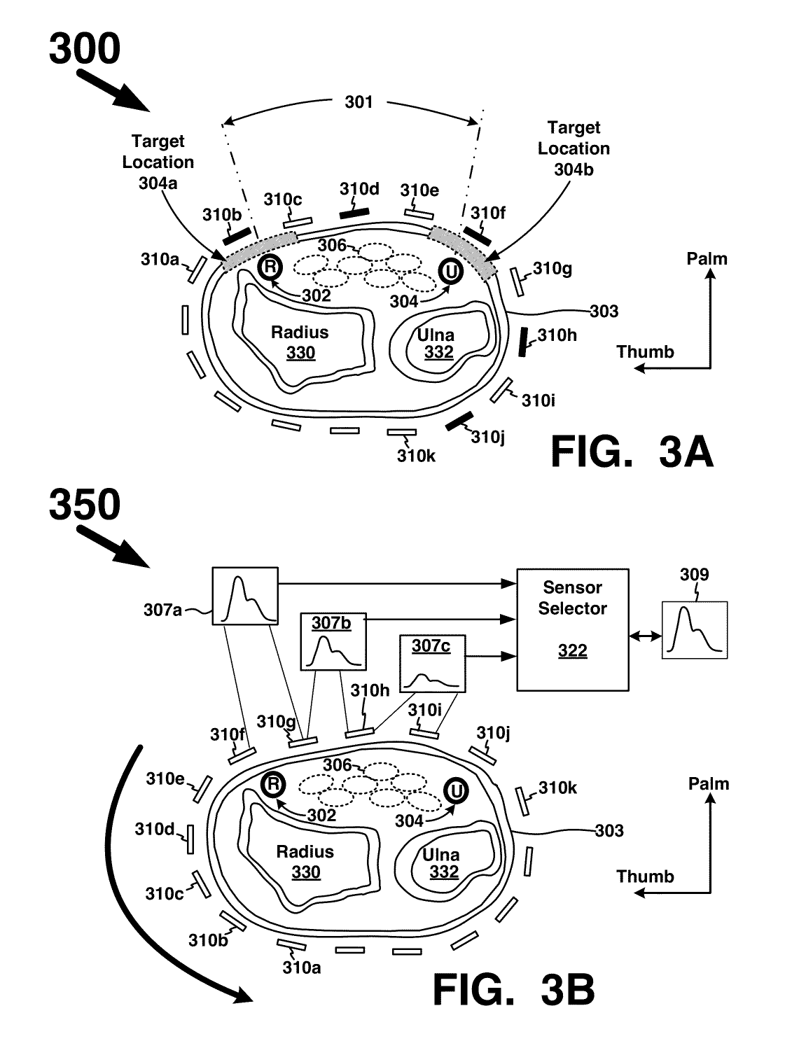

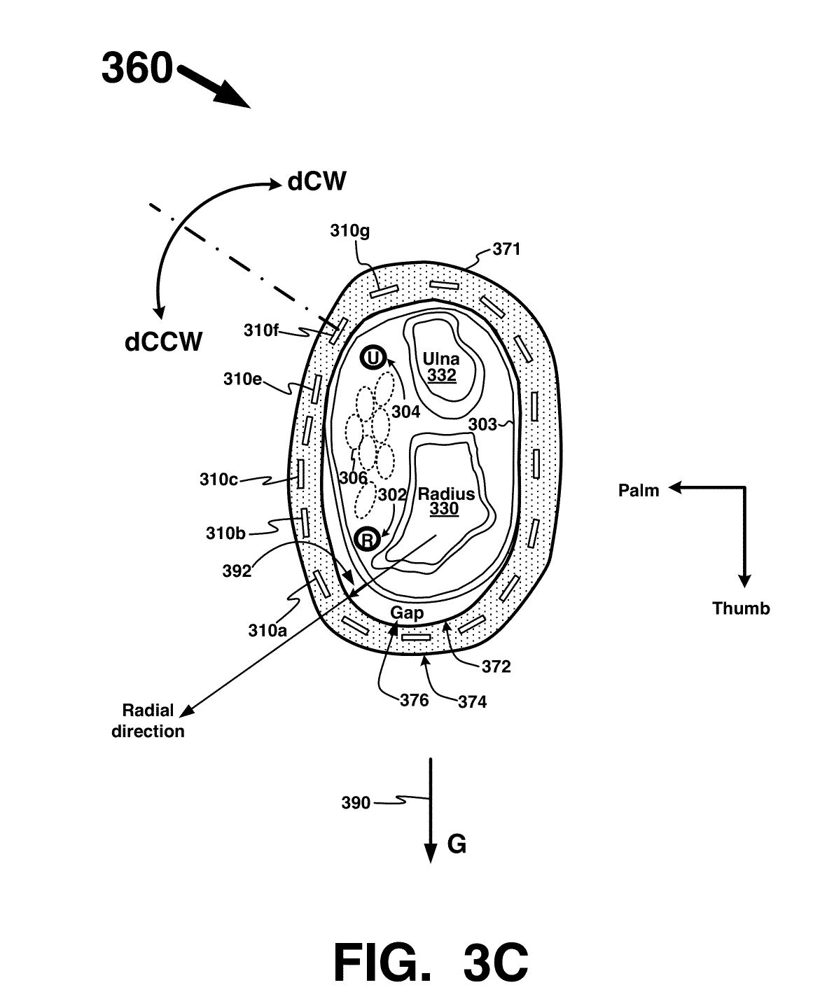

In some instances, a wearable device 170 can select multiple subsets of electrodes to enable data capture using a second subset adjacent to a second target location when a first subset adjacent a first target location is unavailable to capture data. For example, a portion of wearable device 170 including the first subset of electrodes 110 (initially adjacent to a first target location) may be displaced to a position farther away in a radial direction away from a blood vessel, such as depicted by a radial distance 392 of FIG. 3C from the skin of the wearer. That is, subset of electrodes 310a and 310b are displaced radially be distance 392. Further to FIG. 3C, the second subset of electrodes 310f and 310g adjacent to the second target location can be closer in a radial direction toward another blood vessel, and, thus, the second subset of electrodes can acquire physiological characteristics when the first subset of electrodes cannot. Referring back to FIG. 1A, array 101 of electrodes 110 facilitates a wearable device 170 that need not be affixed firmly to the wearer. That is, wearable device 170 can be attached to a portion of the wearer in a manner in which wearable device 170 can be displaced relative to a reference point affixed to the wearer and continue to acquire and generate information regarding physiological characteristics. In some examples, wearable device 170 can be described as being “loosely fitting” on or “floating” about a portion of the wearer, such as a wrist, whereby array 101 has sufficient sensors points from which to pick up physiological signals.

In addition, accelerometers 160 can be used to replace the implementation of subsets of electrodes to detect motion associated with pulsing blood flow, which, in turn, can be indicative of whether oxygen-rich blood is present or not present. Or, accelerometers 160 can be used to supplement the data generated by acquired one or more bioimpedance signals acquired by array 101. Accelerometers 160 can also be used to determine the orientation of wearable device 170 and relative movement of the same to determine or predict a target location. Sensor selector 122 can use the predicted target location to begin the selection of the optimal subsets of electrodes 110, which likely decreases the time to identify a target location. Electrodes 110 of array 101 can be disposed within a material constituting, for example, a housing, according to some embodiments. Therefore, electrodes 110 can be protected from the environment and, thus, need not be subject to corrosive elements. In some examples, one or more electrodes 110 can have at least a portion of a surface exposed. As electrodes 110 of array 101 are configured to couple capacitively to a target location, electrodes 110 thereby facilitate high impedance signal coupling so that the first and second signals can pass through fabric and hair. As such, electrodes 110 need not be limited to direct contact with the skin of a wearer. Further, array 101 of electrodes 110 need not circumscribe a limb or source of physiological characteristics. An array 101 can be linear in nature, or can configurable to include linear and curvilinear portions.

In some embodiments, wearable device 170 can be in communication (e.g., wired or wirelessly) with a mobile device 180, such as a mobile phone or computing device. In some cases, mobile device 180, or any networked computing device (not shown) in communication with wearable device 170 or mobile device 180, can provide at least some of the structures and/or functions of any of the features described herein. As depicted in FIG. 1A and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1A (or any subsequent figure) can represent one or more algorithms. Or, at least one of the elements can represent a portion of logic including a portion of hardware configured to provide constituent structures and/or functionalities.

For example, physiological information generator 120 and any of its one or more components, such as sensor selector 122, motion artifact reduction unit 124, and physiological characteristic determinator 126, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1A (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological information generator 120, including one or more components, such as sensor selector 122, motion artifact reduction unit 124, and physiological characteristic determinator 126, can be implemented in one or more computing devices that include one or more circuits. Thus, at least one of the elements in FIG. 1A (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

FIGS. 1B to 1D illustrate examples of electrode arrays, according to some embodiments. Diagram 130 of FIG. 1B depicts an array 132 that includes sub-arrays 133a, 133b, and 133c of electrodes 110 that are configured to generate data that represent one or more characteristics associated with a user associated with array 132. In various embodiments, drive electrodes and sink electrodes can be disposed in the same sub-array or in different sub-arrays. Note that arrangements of sub-arrays 133a, 133b, and 133c can denote physical or spatial orientations and need not imply electrical, magnetic, or cooperative relationships among electrodes 110 within each sub-array. For example, drive electrode (“D”) 110f can be configured in sub-array 133a as a drive electrode to drive a signal to sink electrode (“S”) 110g in sub-array 133b. As another example, drive electrode (“D”) 110h can be configured in sub-array 133a to drive a signal to sink electrode (“S”) 110k in sub-array 133c. In some embodiments, distances between electrodes 110 in sub-arrays can vary at different regions, including a region in which the placement of electrode group 134 near blood vessel 102 is more probable relative to the placement of other electrodes near blood vessel 102. Electrode group 134 can include a higher density of electrodes 110 than other portions of array 132 as group 134 can be expected to be disposed adjacent blood vessel 102 more likely than other groups of electrodes 110. For example, an elliptical-shaped array (not shown) can be disposed in device 170 of FIG. 1A. Therefore, group 134 of electrodes is disposed at a region 172 of FIG. 1A, which is likely adjacent either a radial artery or an ulna artery. While three sub-arrays are shown, more or fewer are possible.

Referring to FIG. 1C, diagram 140 depicts an array 142 oriented at any angle (“θ”) 144 to an axial line coincident with or parallel to blood vessel 102. Therefore, an array 142 of electrodes need not be oriented orthogonally in each implementation; rather array 142 can be oriented at angles between 0 and 90 degrees, inclusive thereof. In a specific embodiment, an array 146 can be disposed parallel (or substantially parallel) to blood vessel 102a (or a portion thereof).

FIG. 1D is a diagram 150 depicting a wearable device 170a including a helically-shaped array 152 of electrodes disposed therein, whereby electrodes 110m and 110n can be configured as a pair of drive and sink electrodes. As shown, electrodes 110m and 110n substantially align in a direction parallel to an axis 151, which can represent a general direction of blood flow through a blood vessel.

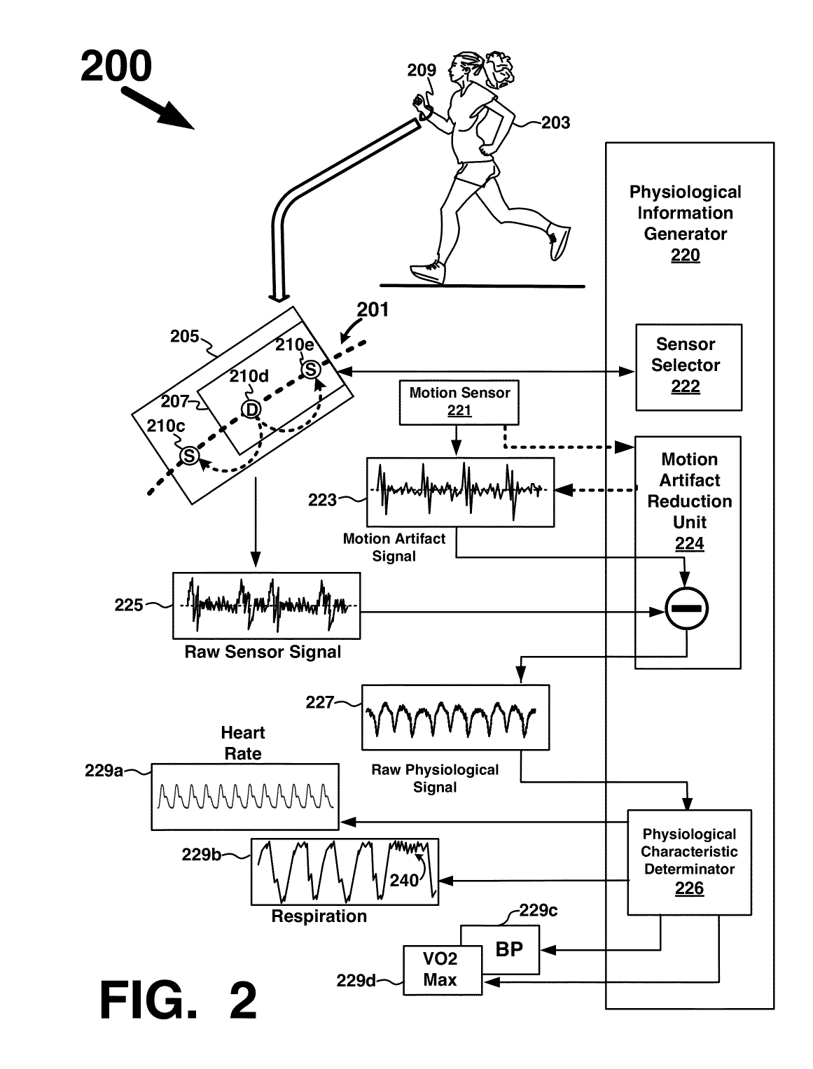

FIG. 2 is a functional diagram depicting a physiological information generator implemented in a wearable device, according to some embodiments. Functional diagram 200 depicts a user 203 wearing a wearable device 209, which includes a physiological information generator 220 configured to generate signals including data representing physiological characteristics. As shown, sensor selector 222 is configured to select a subset 205 of electrodes or a subset 207 of electrodes. Subset 205 of electrodes includes electrodes 210c, 210d, and 210e, and subset 207 of electrodes includes electrodes 210d and 210e. For purposes of illustration, consider that sensor selector 222 selects electrodes 210d and 210c as a subset of electrodes with which to capture physiological characteristics adjacent a target location. Sensor selector 222 applies an AC signal, as a first signal, into electrodes 210d to generate a sensor signal (“raw sensor signal”) 225, as a second signal, from electrode 210c. Sensor signal 222 includes a motion-related signal component and a physiological-related signal component. A motion sensor 221 is configured to capture generate a motion artifact signal 223 based on motion data representing motion experienced by wearable device 209 (or at least the electrodes). A motion artifact reduction unit 224 is configured to receive sensor signal 225 and motion artifact signal 223. Motion artifact reduction unit 224 operates to subtract motion artifact signal 223 from sensor signal 225 to yield the physiological-related signal component (or an approximation thereof) as a raw physiological signal 227. In some examples, raw physiological signal 227 represents an unamplified, unfiltered signal including data representative of one or more physiological characteristics. In some embodiments, motion sensor 221 generates motion signals, such as accelerometer signals. These signals are provided to motion artifact reduction unit 224 (e.g., via dashed lines as shown), which, in turn, is configured to determine motion artifact signal 223. In some embodiments, motion artifact signal 223 represents motion included or embodied within raw sensor signal 225 (e.g., with physiological signal(s)). Thus, a motion artifact signal can describe a motion signal, whether sensed by a motion sensor or integrated with one or more physiological signals. A physiological characteristic determinator 226 is configured to receive raw physiological signal 227 to amplify and/or filter different physiological signal components from raw physiological signal 227. For example, raw physiological signal 227 may include a respiration signal modulated on (or in association with) a heart rate (“HR”) signal. Regardless, physiological characteristic determinator 226 is configured to perform digital signal processing to generate a heart rate (“HR”) signal 229a and/or a respiration signal 229b. Portion 240 of respiration signal 229b represents an impedance signal due to cardiac activity, at least in some instances. Further, physiological characteristic determinator 226 is configured to use either HR signal 229a or a respiration signal 229b, or both, to derive other physiological characteristics, such as blood pressure data (“BP”) 229c, a maximal oxygen consumption (“VO2 max”) 229d, or any other physiological characteristic.

Physiological characteristic determinator 226 can derive other physiological characteristics using other data generated or accessible by wearable device 209, such as the type of activity the wear is engaged, environmental factors, such as temperature, location, etc., whether the wearer is subject to any chronic illnesses or conditions, and any other health or wellness-related information. For example, if the wearer is diabetic or has Parkinson's disease, motion sensor 221 can be used to detect tremors related to the wearer's ailment. With the detection of small, but rapid movements of a wearable device that coincide with a change in heart rate (e.g., a change in an HR signal) and/or breathing, physiological information generator 220 may generate data (e.g., an alarm) indicating that the wearer is experiencing tremors. For a diabetic, the wearer may experience shakiness because the blood-sugar level is extremely low (e.g., it drops below a range of 38 to 42 mg/dl). Below these levels, the brain may become unable to control the body. Moreover, if the arms of a wearer shakes with sufficient motion to displace a subset of electrodes from being adjacent a target location, the array of electrodes, as described herein, facilitates continued monitoring of a heart rate by repeatedly selecting subsets of electrodes that are positioned optimally (e.g., adjacent a target location) for receiving robust and accurate physiological-related signals.

FIGS. 3A to 3C are cross-sectional views depicting arrays of electrodes including subsets of electrodes adjacent an arm portion of a wearer, according to some embodiments. Diagram 300 of FIG. 3A depicts an array of electrodes arranged about, for example, a wrist of a wearer. In this cross-sectional view, an array of electrodes includes electrodes 310a, 310b, 310c, 310d, 310e, 310f, 310g, 310h, 310i, 310j, and 310k, among others, arranged about wrist 303 (or the forearm). The cross-sectional view of wrist 303 also depicts a radius bone 330, an ulna bone 332, flexor muscles/ligaments 306, a radial artery (“R”) 302, and an ulna artery (“U”) 304. Radial artery 302 is at a distance 301 (regardless of whether linear or angular) from ulna artery 304. Distance 301 may be different, on average, for different genders, based on male and female anatomical structures. Notably, the array of electrodes can obviate specific placement of electrodes due to different anatomical structures based on gender, preference of the wearer, issues associated with contact (e.g., contact alignment), or any other issue that affects placement of electrode that otherwise may not be optimal. To effect appropriate electrode selection, a sensor selector, as described herein, can use gender-related information (e.g., whether the wearer is male or female) to predict positions of subsets of electrodes such that they are adjacent (or substantially adjacent) to one or more target locations 304a and 304b. Target locations 304a and 304b represent optimal areas (or volumes) at which to measure, to monitor and to capture data related to bioimpedances. In particular, target location 304a represents an optimal area adjacent radial artery 302 to pick up bioimpedance signals, whereas target location 304b represents another optimal area adjacent ulna artery 304 to pick up other bioimpedance signals.

To illustrate the resiliency of a wearable device to maintain an ability to monitor physiological characteristics over one or more displacements of the wearable device (e.g., around or along wrist 303), consider that a sensor selector configures initially electrodes 310b, 310d, 310f, 310h, and 310j as driver electrodes and electrodes 310a, 310c, 310e 310g, 310i, and 310k as sink electrodes. Further consider that the sensor selector identifies a first subset of electrodes that includes electrodes 310b and 310c as a first optimal subset, and also identifies a second subset of electrodes that include electrodes 310f and 310g as a second optimal subset. Note that electrodes 310b and 310c are adjacent target location 304a and electrodes 310f and 310g are adjacent to target location 304b. These subsets are used to periodically (or aperiodically) monitor the signals from electrodes 310c and 310g, until the first and second subsets are no longer optimal (e.g., when movement of the wearable device displaces the subsets relative to the target locations). Note that the functionality of driver and sink electrodes for electrodes 310b, 310c, 310f, and 310g can be reversed (e.g., electrodes 310a and 310g can be configured as drive electrodes).

FIG. 3B depicts an array of FIG. 3A being displaced from an initial position, according to some examples. In particular, diagram 350 depicts that electrodes 310f and 310g are displaced to a location adjacent radial artery 302 and electrodes 310j and 310k are displaced to a location adjacent ulna artery 304. According to some embodiments, a sensor selector 322 is configured to test subsets of electrodes to determine at least one subset, such as electrodes 310f and 310, being located adjacent to a target location (next to radial artery 302). To identify electrodes 310f and 310g as an optimal subset, sensor selector 322 is configured to apply drive signals to the drive electrodes to generate a number of data samples, such as data samples 307a, 307b, and 307c. In this example, each data sample represents a portion of a physiological characteristic, such as a portion of an HR signal. Sensor selector 322 operates to compare the data samples against a profile 309 to determine which of data samples 307a, 307b, and 307c best fits or is comparable to a predefined set of data represented by profile data 309. Profile data 309, in this example, represents an expected HR portion or thresholds indicating a best match. Also, profile data 309 can represent the most robust and accurate HR portion measured during the sensor selection mode relative to all other data samples (e.g., data sample 307a is stored as profile data 309 until, and if, another data sample provides a more robust and/or accurate data sample). As shown, data sample 307a substantially matches profile data 309, whereas data samples 307b and 307c are increasingly attenuated as distances increase away from radial artery 302. Therefore, sensor selector 322 identifies electrodes 310f and 310g as an optimal subset and can use this subset in data capture mode to monitor (e.g., continuously) the physiological characteristics of the wearer. Note that the nature of data samples 307a, 307b, and 307c as portions of an HR signal is for purposes of explanation and is not intended to be limiting. Data samples 307a, 307b, and 307c need not be portions of a waveform or signal, and need not be limited to an HR signal. Rather, data samples 307a, 307b, and 307c can relate to a respiration signal, a raw sensor signal, a raw physiological signal, or any other signal. Data samples 307a, 307b, and 307c can represent a measured signal attribute, such as magnitude or amplitude, against which profile data 309 is matched. In some cases, an optimal subset of electrodes can be associated with a least amount of impedance and/or reactance (e.g., over a period of time) when applying a first signal (e.g., a drive signal) to a target location

FIG. 3C depicts an array of electrodes of FIG. 3A oriented differently due to a change in orientation of a wrist of a wearer, according to some examples. In this example, the array of electrodes is shown to be disposed in a wearable device 371, which has an outer surface 374 and an inner surface 372. In some embodiments, wearable device 371 can be configured to “loosely fit” around the wrist, thereby enabling rotation about the wrist. In some cases, a portion of wearable devices 371 (and corresponding electrodes 310a and 310b) are subject to gravity (“G”) 390, which pulls the portion away from wrist 303, thereby forming a gap 376. Gap 376, in turn, causes inner surface 372 and electrodes 310a and 310b to be displaced radially by a radial distance 392 (i.e., in a radial direction away from wrist 303). Gap 376, in some cases, can be an air gap. Radial distance 392, at least in some cases, may impact electrodes 310a and 310b and the ability to receive signals adjacent to radial artery 302. Regardless, electrodes 310f and 310g are positioned in another portion of wearable device 371 and can be used to receive signals adjacent to ulna artery 304 in cooperation with, or instead of, electrodes 310a and 310b. Therefore, electrodes 310f and 310g (or any other subset of electrodes) can provide redundant data capturing capabilities should other subsets be unavailable.

Next, consider that sensor selector 322 of FIG. 3B is configured to determine a position of electrodes 310f and 310g (e.g., on the wearable device 371) relative to a direction of gravity 390. A motion sensor (not shown) can determine relative movements of the position of electrodes 310f and 310g over any number of movements in either a clockwise direction (“dCW”) or a counterclockwise direction (“dCCW”). As wearable device 371 need not be affixed firmly to wrist 303, at least in some examples, the position of electrodes 310f and 310g may “slip” relative to the position of ulna artery 304. In one embodiment, sensor selector 322 can be configured to determine whether another subset of electrodes are optimal, if electrodes 310f and 310g are displaced farther away than a more suitable subset. In sensor selecting mode, sensor selector 322 is configured to select another subset, if necessary, by beginning the capture of data samples at electrodes 310f and 310g and progressing to other nearby subsets to either confirm the initial selection of electrodes 310f and 310g or to select another subset. In this manner, the identification of the optimal subset may be determined in less time than if the selection process is performed otherwise (e.g., beginning at a specific subset regardless of the position of the last known target location).

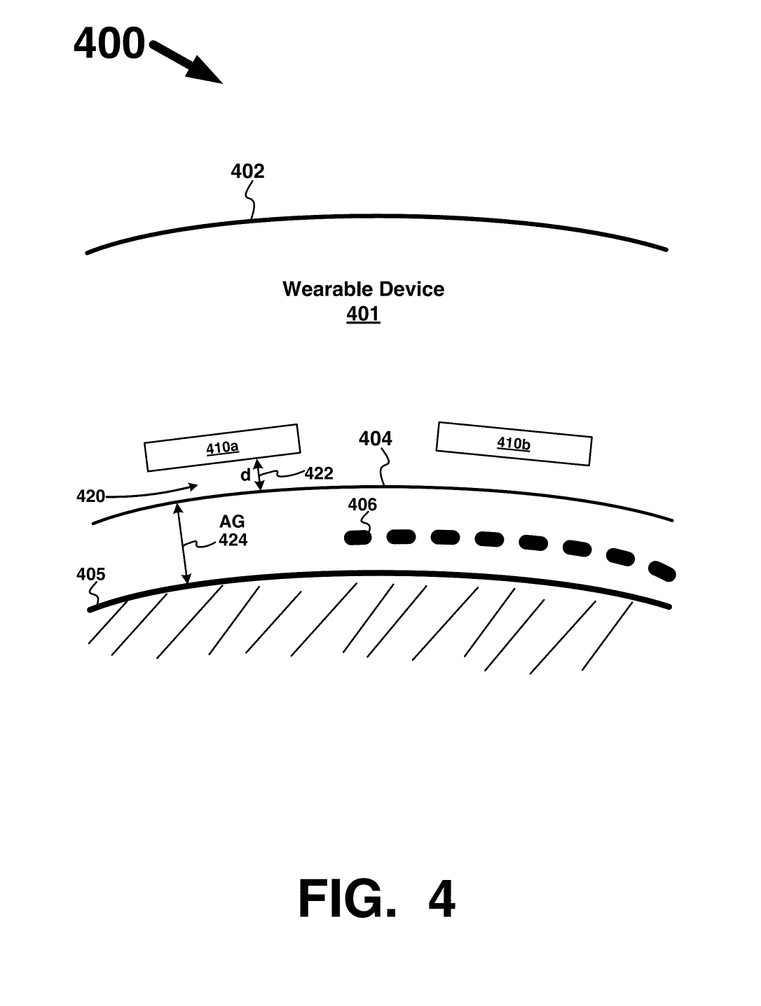

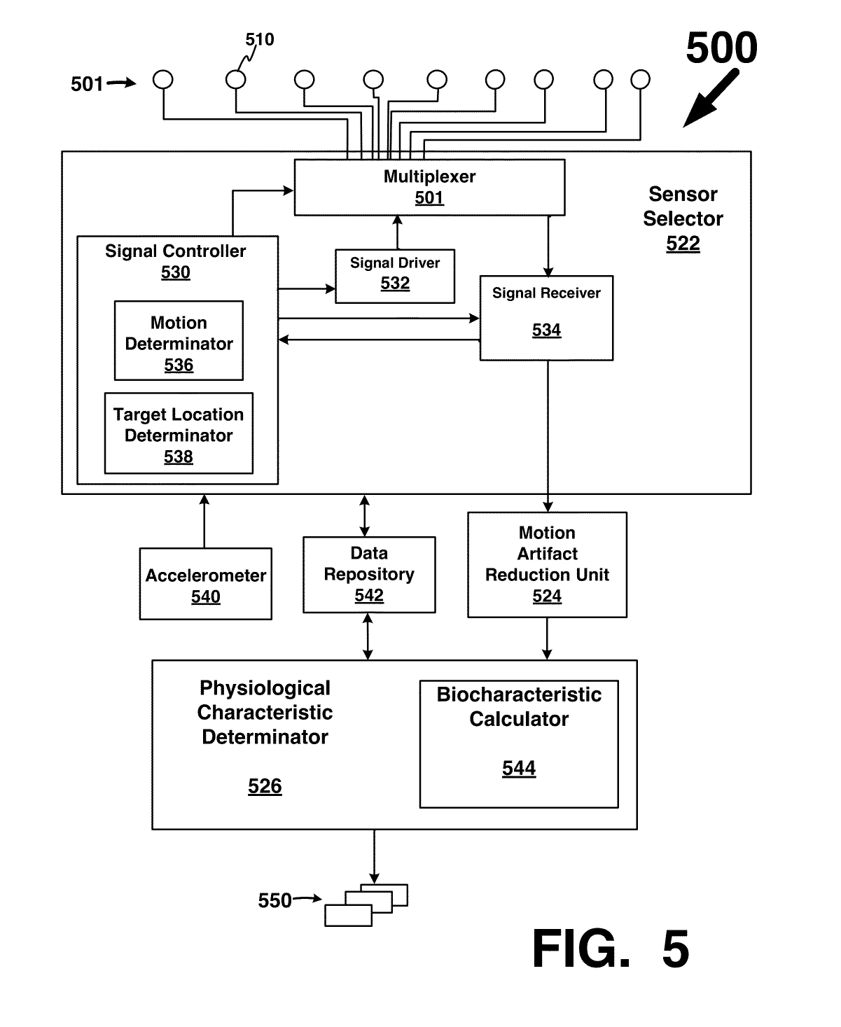

FIG. 4 depicts a portion of an array of electrodes disposed within a housing material of a wearable device, according to some embodiments. Diagram 400 depicts electrodes 410a and 410b disposed in a wearable device 401, which has an outer surface 402 and an inner surface 404. In some embodiments, wearable device 401 includes a material in which electrodes 410a and 410b can be encapsulated in a material to reduce or eliminate exposure to corrosive elements in the environment external to wearable device 401. Therefore, material 420 is disposed between the surfaces of electrodes 410a and 410b and inner surface 404. Driver electrodes are capacitively coupled to skin 405 to transmit high impedance signals, such as a current signal, over distance (“d”) 422 through the material, and, optionally, through fabric 406 or hair into skin 405 of the wearer. Also, the current signal can be driven through an air gap (“AG”) 424 between inner surface 404 and skin 405. Note that in some implementations, electrodes 410a and 410b can be exposed (or partially exposed) out through inner surface 404. In some embodiments, electrodes 410a and 410b can be coupled via conductive materials, such as conductive polymers or the like, to the external environment of wearable device 401.

FIG. 5 depicts an example of a physiological information generator, according to some embodiments. Diagram 500 depicts an array 501 of electrodes 510 that can be disposed in a wearable device. A physiological information generator can include one or more of a sensor selector 522, an accelerometer 540 for generating motion data, a motion artifact reduction unit 524, and a physiological characteristic determinator 526. Sensor selector 522 includes a signal controller 530, a multiplexer 501 (or equivalent switching mechanism), a signal driver 532, a signal receiver 534, a motion determinator 536, and a target location determinator 538. Sensor selector 522 is configured to operate in at least two modes. First, sensor selector 522 can select a subset of electrodes in a sensor select mode of operation. Second, sensor selector 522 can use a selected subset of electrodes to acquire physiological characteristics, such as in a data capture mode of operation, according to some embodiments. In sensor select mode, signal controller 530 is configured to serially (or in parallel) configure subsets of electrodes as driver electrodes and sink electrodes, and to cause multiplexer 501 to select subsets of electrodes 510. In this mode, signal driver 532 applies a drive signal via multiplexer 501 to a selected subset of electrodes, from which signal receiver 534 receives via multiplexer 501a sensor signal. Signal controller 530 acquires a data sample for the subset under selection, and then selects another subset of electrodes 510. Signal controller 530 repeats the capture of data samples, and is configured to determine an optimal subset of electrodes for monitoring purposes. Then, sensor selector 522 can operate in the data capture mode of operation in which sensor selector 522 continuously (or substantially continuously) captures sensor signal data from at least one selected subset of electrodes 501 to identify physiological characteristics in real time (or in near real-time).

In some embodiments, a target location determinator 538 is configured to initiate the above-described sensor selection mode to determine a subset of electrodes 510 adjacent a target location. Further, target location determinator 538 can also track displacements of a wearable device in which array 501 resides based on motion data from accelerometer 540. For example, target location determinator 538 can be configured to determine an optimal subset if the initially-selected electrodes are displaced farther away from the target location. In sensor selecting mode, target location determinator 538 can be configured to select another subset, if necessary, by beginning the capture of data samples at electrodes for the last known subset adjacent to the target location, and progressing to other nearby subsets to either confirm the initial selection of electrodes or to select another subset. In some examples, orientation of the wearable device, based on accelerometer data (e.g., a direction of gravity), also can be used to select a subset of electrodes 501 for evaluation as an optimal subset. Motion determinator 536 is configured to detect whether there is an amount of motion associated with a displacement of the wearable device. As such, motion determinator 536 can detect motion and generate a signal to indicate that the wearable device has been displaced, after which signal controller 530 can determine the selection of a new subset that is more closely situated near a blood vessel than other subsets, for example. Also, motion determinator 536 can cause signal controller 530 to disable data capturing during periods of extreme motion (e.g., during which relatively large amounts of motion artifacts may be present) and to enable data capturing during moments when there is less than an extreme amount of motion (e.g., when a tennis player pauses before serving). Data repository 542 can include data representing the gender of the wearer, which is accessible by signal controller 530 in determining the electrodes in a subset.

In some embodiments, signal driver 532 may be a constant current source including an operational amplifier configured as an amplifier to generate, for example, 100 μA of alternating current (“AC”) at various frequencies, such as 50 kHz. Note that signal driver 532 can deliver any magnitude of AC at any frequency or combinations of frequencies (e.g., a signal composed of multiple frequencies). For example, signal driver 532 can generate magnitudes (or amplitudes), such as between 50 μA and 200 μA, as an example. Also, signal driver 532 can generate AC signals at frequencies from below 10 kHz to 550 kHz, or greater. According to some embodiments, multiple frequencies may be used as drive signals either individually or combined into a signal composed of the multiple frequencies. In some embodiments, signal receiver 534 may include a differential amplifier and a gain amplifier, both of which can include operational amplifiers.

Motion artifact reduction unit 524 is configured to subtract motion artifacts from a raw sensor signal received into signal receiver 534 to yield the physiological-related signal components for input into physiological characteristic determinator 526. Physiological characteristic determinator 526 can include one or more filters to extract one or more physiological signals from the raw physiological signal that is output from motion artifact reduction unit 524. A first filter can be configured for filtering frequencies for example, between 0.8 Hz and 3 Hz to extract an HR signal, and a second filter can be configured for filtering frequencies between 0 Hz and 0.5 Hz to extract a respiration signal from the physiological-related signal component. Physiological characteristic determinator 526 includes a biocharacteristic calculator that is configured to calculate physiological characteristics 550, such as VO2 max, based on extracted signals from array 501.

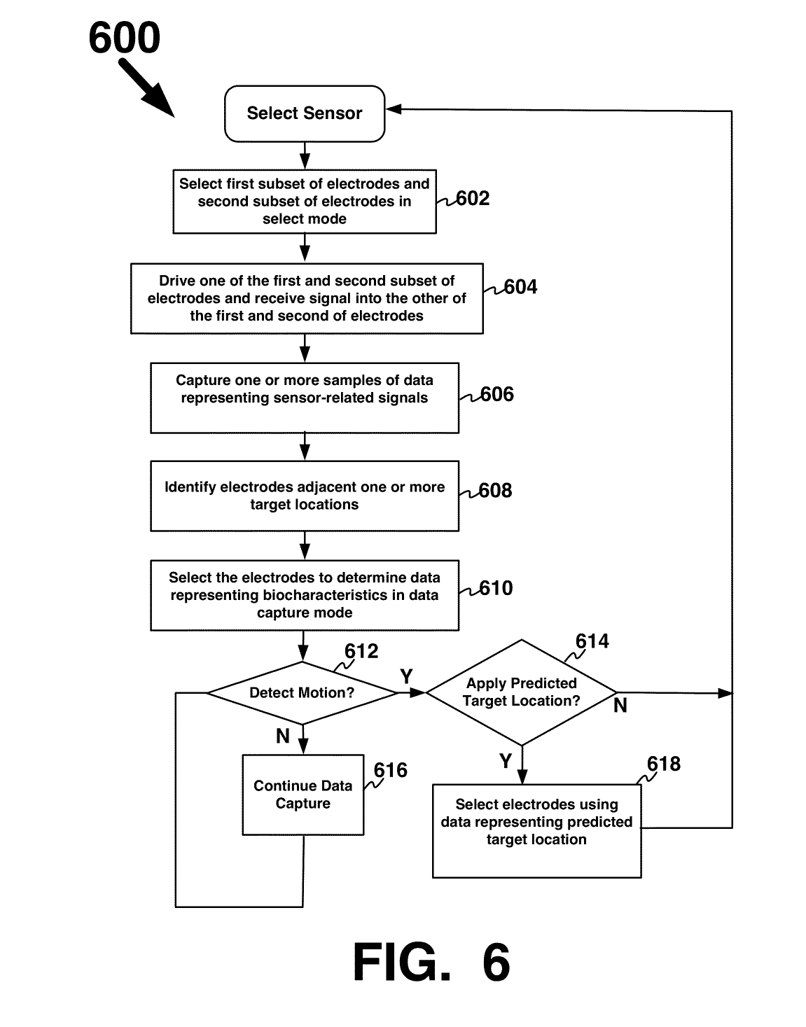

FIG. 6 is an example flow diagram for selecting a sensor, according to some embodiments. At 602, flow 600 provides for the selection of a first subset of electrodes and the selection of a second subset of electrodes in a select sensor mode. At 604, one of the first and second subset of electrodes is selected as a drive electrode and the other of the first and second subset of electrodes is selected as a sink electrode. In particular, the first subset of electrodes can, for example, include one or more drive electrodes, and the second subset of electrodes can include one or more sink electrodes. At 606, one or more data samples are captured, the data samples representing portions of a measured signal (or values thereof). Based on a determination that one of the data samples is indicative of a subset of electrodes adjacent a target location, the electrodes of the optimal subset are identified at 608. At 610, the identified electrodes are selected to capture signals including physiological-relate components. While there is no detected motion at 612, flow 600 moves to 616 to capture, for example, heart and respiration data continuously. When motion is detected at 612, data capture may continue. But flow 600 moves to 614 to determine whether to apply a predicted target location. In some cases, a predicted target location is based on the initial target location (e.g., relative to the initially-determined subset of electrodes), with subsequent calculations based on amounts and directions of displacement, based on accelerometer data, to predict a new target location. One or more motion sensors can be used to determine the orientation of a wearable device, and relative movement of the same (e.g., over a period of time or between events), to determine or predict a target location. Or, the predicted target location can refer to the last known target location and/or subset of electrodes. At 618, electrodes are selected based on the predicted target location for confirming whether the previously-selected subset of electrodes are optimal, or whether a new, optimal subset is to be determined as flow 600 moves back to 602.

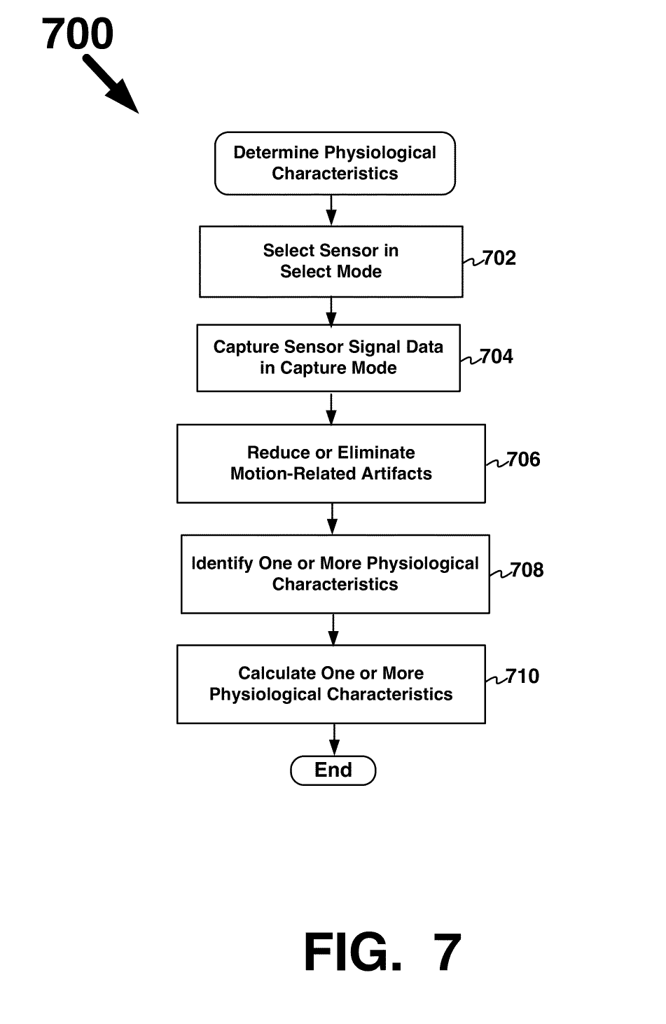

FIG. 7 is an example flow diagram for determining physiological characteristics using a wearable device with arrayed electrodes, according to some embodiments. At 702, flow 700 provides for the selection of a sensor in sensor select mode, the sensor including, for example, two or more electrodes. At 704, sensor signal data is captured in data capture mode. At 706, motion-related artifacts can be reduced or eliminated from the sensor signal to yield a physiological-related signal component. One or more physiological characteristics can be identified at 708, for example, after digitally processing the physiological-related signal component. At 710, one or more physiological characteristics can be calculated based on the data signals extracted at 708. Examples of calculated physiological characteristics include maximal oxygen consumption (“VO2 max”).

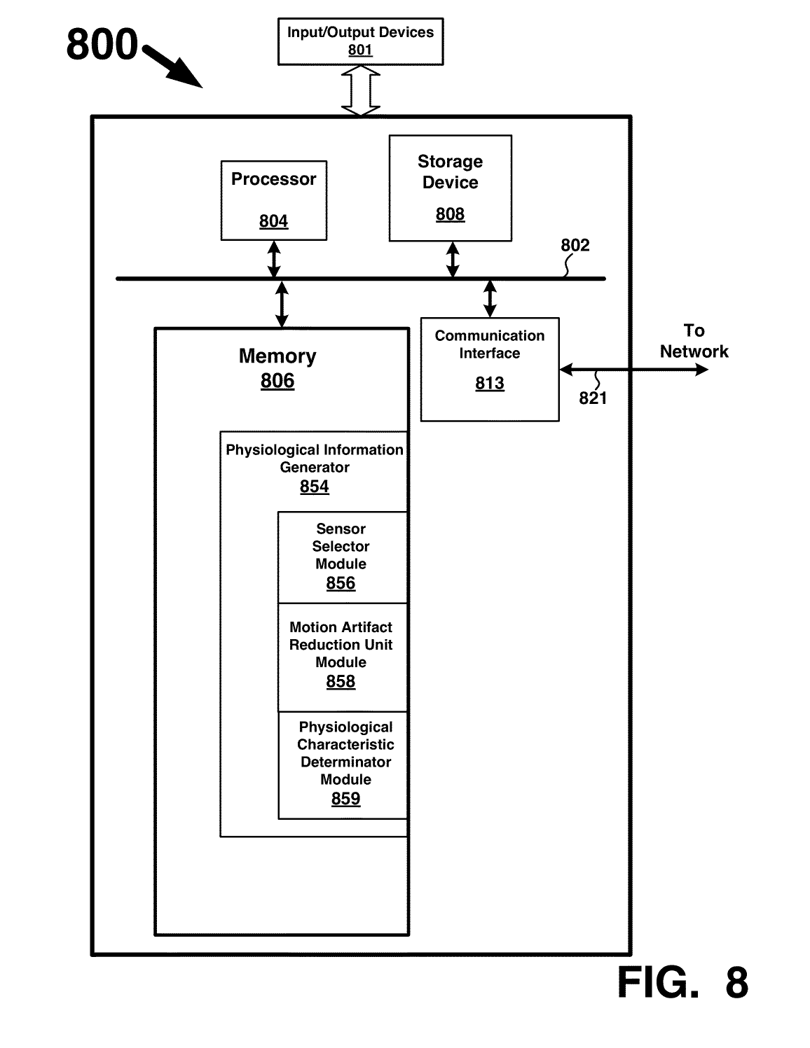

FIG. 8 illustrates an exemplary computing platform disposed in a wearable device in accordance with various embodiments. In some examples, computing platform 800 may be used to implement computer programs, applications, methods, processes, algorithms, or other software to perform the above-described techniques. Computing platform 800 includes a bus 802 or other communication mechanism for communicating information, which interconnects subsystems and devices, such as processor 804, system memory 806 (e.g., RAM, etc.), storage device 808 (e.g., ROM, etc.), a communication interface 813 (e.g., an Ethernet or wireless controller, a Bluetooth controller, etc.) to facilitate communications via a port on communication link 821 to communicate, for example, with a computing device, including mobile computing and/or communication devices with processors. Processor 804 can be implemented with one or more central processing units (“CPUs”), such as those manufactured by Intel® Corporation, CircuitCo Printed Circuit Board Solutions, or one or more virtual processors, as well as any combination of CPUs and virtual processors. Computing platform 800 exchanges data representing inputs and outputs via input-and-output devices 801, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 800 performs specific operations by processor 804 executing one or more sequences of one or more instructions stored in system memory 806, and computing platform 800 can be implemented in a client-server arrangement, peer-to-peer arrangement, or as any mobile computing device, including smart phones and the like. Such instructions or data may be read into system memory 806 from another non-transitory computer readable medium, such as storage device 808. In some examples, hard-wired circuitry may be used in place of or in combination with software instructions for implementation. Instructions may be embedded in software or firmware. The term “non-transitory computer readable medium” refers to any tangible medium that participates in providing instructions to processor 804 for execution. Such a medium may take many forms, including but not limited to, non-volatile media and volatile media. Non-volatile media includes, for example, optical or magnetic disks and the like. Volatile media includes dynamic memory, such as system memory 806.

Common forms of non-transitory computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 802 for transmitting a computer data signal.

In some examples, execution of the sequences of instructions may be performed by computing platform 800. According to some examples, computing platform 800 can be coupled by communication link 821 (e.g., a wired network, such as LAN, PSTN, or any wireless network) to any other processor to perform the sequence of instructions in coordination with (or asynchronous to) one another. Computing platform 800 may transmit and receive messages, data, and instructions, including program code (e.g., application code) through communication link 821 and communication interface 813. Received program code may be executed by processor 804 as it is received, and/or stored in memory 806 or other non-volatile storage for later execution.

In the example shown, system memory 806 can include various modules that include executable instructions to implement functionalities described herein. In the example shown, system memory 806 includes a physiological information generator module 854 configured to implement determine physiological information relating to a user that is wearing a wearable device. Physiological information generator module 854 can include a sensor selector module 856, a motion artifact reduction unit module 858, and a physiological characteristic determinator 859, any of which can be configured to provide one or more functions described herein.

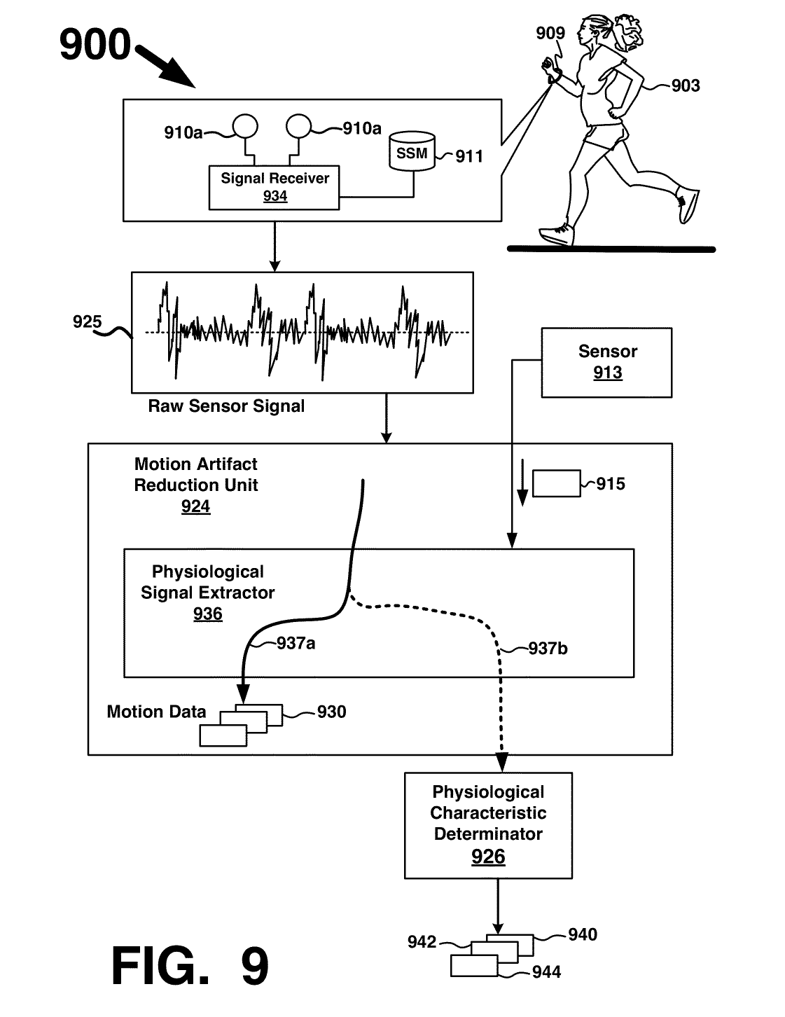

FIG. 9 depicts the physiological signal extractor, according to some embodiments. Diagram 900 depicts a motion artifact reduction unit 924 including a physiological signal extractor 936. In some embodiments, motion artifact reduction unit 924 can be disposed in or attached to a wearable device 909, which can be configured to attached to or otherwise be worn by user 903. As shown, user 903 is running or jogging, whereby movement of the limbs of user 903 imparts forces that cause wearable device 909 to experience motion. Motion artifact reduction unit 924 is configured to receive a sensor signal (“Raw Sensor Signal”) 925, and is further configured to reduce or negate motion artifacts accompanying, or mixed with, physiological signals due to motion-related noise that otherwise affects sensor signal 925. Further to diagram 900, a signal receiver 934 is coupled to a sensor including, for example, one or more electrodes. Examples of such electrodes include electrode 910a and electrode 910b. In some embodiments, signal receiver 934 includes similar structure and/or functionality as signal receiver 534 of FIG. 5. In operation, signal receiver 934 is configured to receive one or more AC current signals, such as high impedance signals, as bioimpedance-related signals. Signal receiver 934 can include differential amplifiers, gain amplifiers, or any other operational amplifier configured to receive, adapt (e.g., amplify), and transmit sensor signal 925 to motion artifact reduction unit 924.

In some embodiments, signal receiver 934 is configured to receive electrical signals representing acoustic-related information from a microphone 911. An example of the acoustic-related information includes data representing a heartbeat or a heart rate as sensed by microphone 911, such that sensor signal 925 can be an electrical signal derived from acoustic energy associated with a sensed physiological signal, such as a pulse wave or heartbeat. Wearable device 909 can include microphone 911 configured to contact (or to be positioned adjacent to) the skin of the wearer, whereby microphone 911 is adapted to receive sound and acoustic energy generated by the wearer (e.g., the source of sounds associated with physiological information). Microphone 911 can also be disposed in wearable device 909. According to some embodiments, microphone 911 can be implemented as a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005, and U.S. patent application Ser. No. 13/672,398, filed on Nov. 8, 2012, both of which are incorporated by reference. As used herein, the term human tissue can refer to, at least in some examples, as skin, muscle, blood, or other tissue. In some embodiments, a piezoelectric sensor can constitute an SSM. Data representing sensor signal 925 can include acoustic signal information received from an SSM or other microphone, according to some examples.

According to some embodiments, physiological signal extractor 936 is configured to receive sensor signal 925 and data representing sensing information 915 from another, secondary sensor 913. In some examples, sensor 913 is a motion sensor (e.g., an accelerometer) configured to sense accelerations in one or more axes and generates motion signals indicating an amount of motion and/or acceleration. Note, however, that sensor 913 need not be so limited and can be any other sensor. Examples of suitable sensors are disclosed in U.S. Non-Provisional patent application Ser. No. 13/492,857, filed on Jun. 9, 2012, which is incorporated by reference. Further, physiological signal extractor 936 is configured to operate to identify a pattern (e.g., a motion “signature”), based on motion signal data generated by sensor 913, that can used to decompose sensor signal 925 into motion signal components 937a and physiological signal components 937b. As shown, motion signal components 937a and physiological signal components 937b can correspondingly be used by motion artifact reduction unit 924, or any other structure and/or function described herein, to form motion data 930 and one or more physiological data signals, such as physiological characteristic signals 940, 942, and 944. Physiological characteristic determinator 926 is configured to receive physiological signal components 937b of a raw physiological signal, and to filter different physiological signal components to form physiological characteristic signal(s). For example, physiological characteristic determinator 926 can be configured to analyze the physiological signal components to determine a physiological characteristic, such as a heartbeat, heart rate, pulse wave, respiration rate, a Mayer wave, and other like physiological characteristic. Physiological characteristic determinator 926 is also configured to generate a physiological characteristic signal that includes data representing the physiological characteristic during one or more portions of a time interval during which motion is present. Examples of physiological characteristic signals include data representing one or more of a heart rate 940, a respiration rate 942, Mayer wave frequencies 944, and any other sensed characteristic, such as a galvanic skin response (“GSR”) or skin conductance. Note that the term “heart rate” can refer, at least in some embodiments, to any heart-related physiological signal, including, but not limited to, heart beats, heart beats per minute (“bpm”), pulse, and the like. In some examples, the term “heart rate” can refer also to heart rate variability (“HRV”), which describes the variation of a time interval between heartbeats. HRV describes a variation in the beat to beat interval and can be described in terms of frequency components (e.g., low frequency and high frequency components), at least in some cases.

In view of the foregoing, the functions and/or structures of motion artifact reduction unit 924, as well as its components and/or neighboring components, can facilitate the extraction and derivation of physiological characteristics in situ—during which a user is engaged in physical activity that imparts motion on a wearable device, whereby biometric sensors, such as electrodes, may receive bioimpedance sensor signals that are exposed to, or include, motion-related artifacts. For example, physiological signal extractor 936 can be configured to receive the sensor signal that includes data representing physical physiological characteristics during one or more portions of the time interval in which the wearable devices is in motion. A user 903 need not be required to remain immobile to determine physiological signal characteristic signals. Therefore, user 903 can receive heart rate information, respiration information, and other physiological information during physical activity or during periods of time in which user 903 is substantially or relatively active. Further, according to various embodiments, physiological signal extractor 936 facilitates the sensing of physiological characteristic signals at a distal end of a limb or appendage, such as at a wrist, of user 903. Therefore, various implementations of motion artifact reduction unit 924 can enable the detection of physiological signal at the extremities of user 903, with minimal or reduced effects of motion-related artifacts and their influence on the desired measured physiological signal. By facilitating the detection of physiological signals at the extremities, wearable device 909 can assist user 903 to detect oncoming ailments or conditions of the person's body (e.g., oncoming tremors, states of sleep, etc.) relative to other portions of the person's body, such as proximal portions of a limb or appendage.

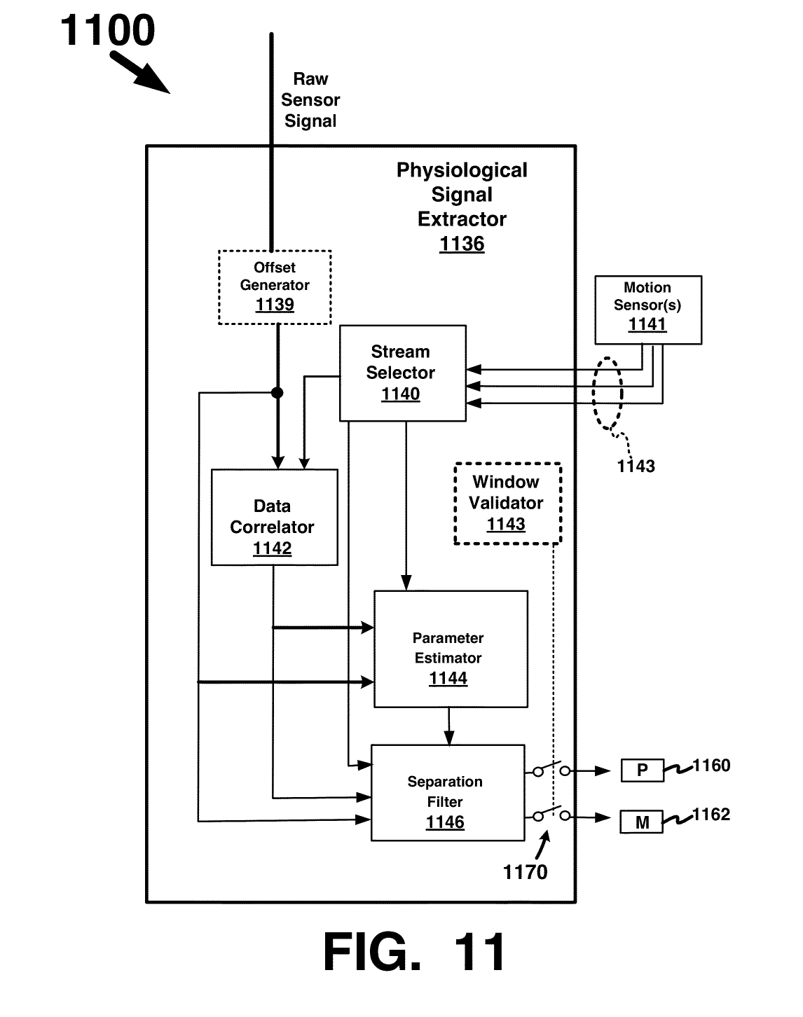

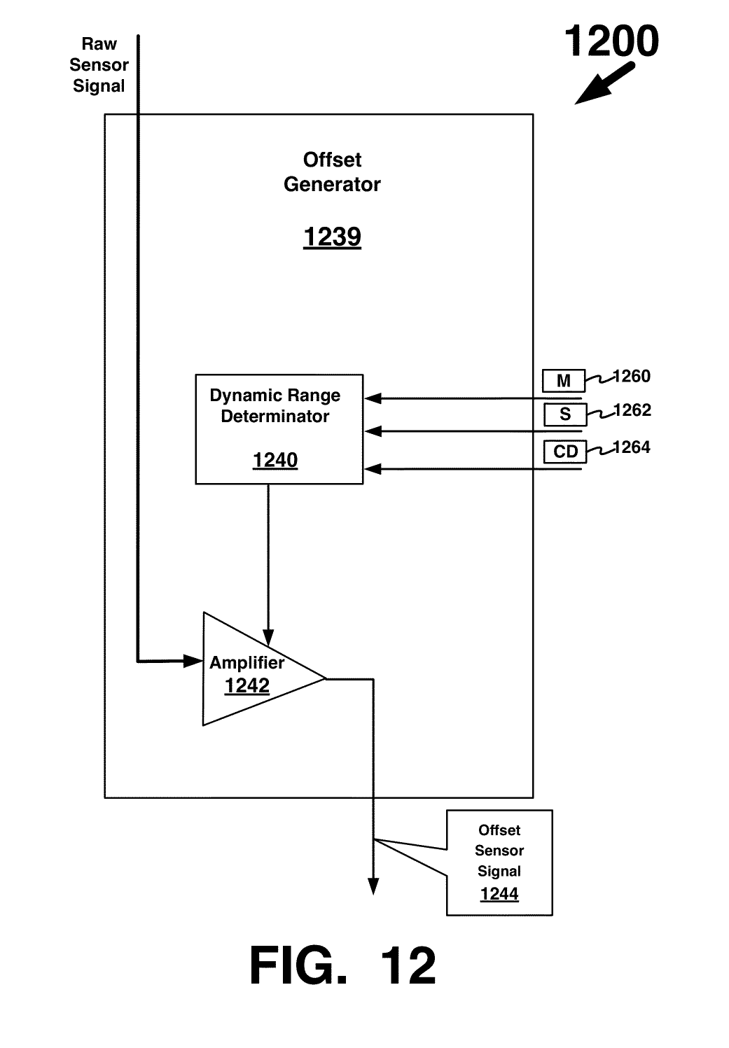

In accordance with some embodiments, physiological signal extractor 936 can include an offset generator, which is not shown. An offset generator can be configured to determine an amount of motion that is associated with the motion sensor signal, such as an accelerometer signal, and to adjust the dynamic range of operation of an amplifier, where the amplifier is configured to receive a sensor signal responsive to the amount of motion. An example of such an amplifier is an operational amplifier configured as a front-end amplifier to enhance, for example, the signal-to-noise ratio. In situations in which the motion related artifacts induce a rapidly-increasing amplitude onto the sensor signal, the amplifier may drive into saturation, which, in turn, causes clipping of the output of the amplifier. The offset generator also is configured to apply in offset value to an amplifier to modify the dynamic range of the amplifier so as to reduce or negate large magnitudes of motion artifacts that may otherwise influence the amplitude of the sensor signal. Examples of an offset generator are described in relation to FIG. 12. In some embodiments, physiological signal extractor 936 can include a window validator configured to determine durations (i.e., a valid window of time) in which sensor signal data can be predicted to be valid (i.e., durations in which the magnitude of motion-related artifacts signals likely do not influence the physiological signals). An example of a window validator is described in FIG. 11.

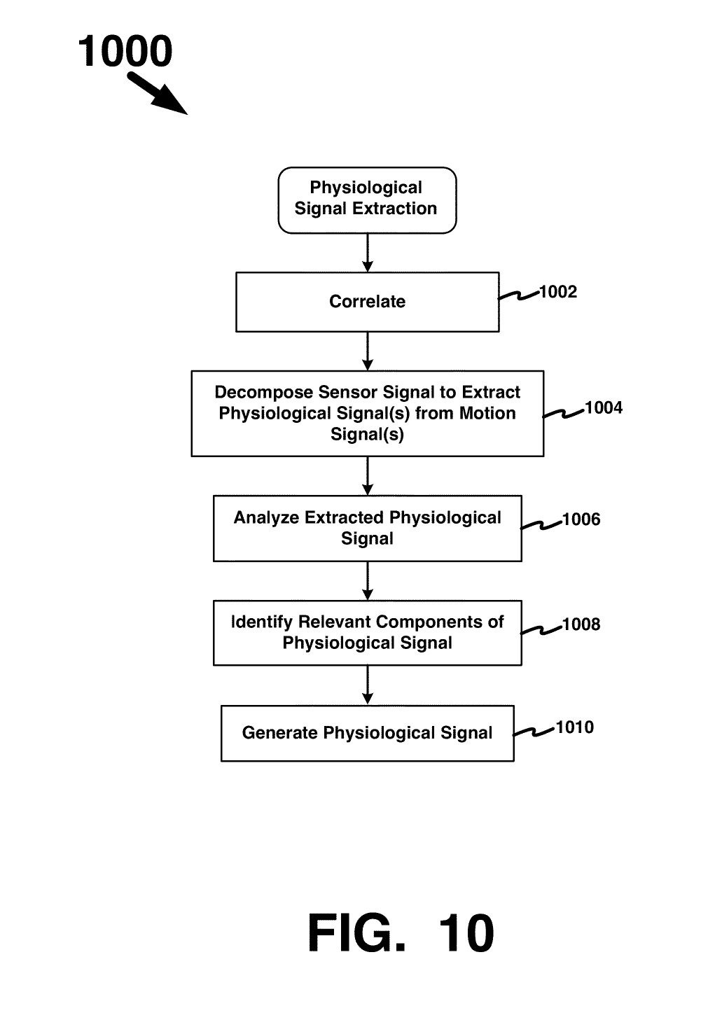

FIG. 10 is a flowchart for extracting a physiological signal, according to some embodiments. At 1002, a motion sensor signal is correlated to a sensor signal, which includes one or more physiological characteristic signals and one or more motion-related artifact signals. In some examples, correlating motion sensor signals to bioimpedance signals enables the two signals to be compared against each other, whereby motion-related artifacts can be subtracted from the bioimpedance signals to extract a physiological characteristic signal. In at least one embodiment, data correlation at 1002 can be performed to include scaling data that represents a motion sensor signal, whereby the scaling facilitates making values for the data representing sensor signal equivalent so that they can be compared against each other (e.g., to facilitate subtracting one signal from the other). At 1004, a sensor signal is decomposed to extract one or more physiological signals and one or more motion sensor signals, thereby separating physiological signals from the motion signals. The extracted physiological signal is analyzed at 1006. In some examples, the frequency of the extracted physiological signal is analyzed to identify a dominant frequency component or predominant frequency components. Also, such an analysis at 1006 can also determine power spectral densities of the physiological extract physiological signal. At 1008, the relevant components of the physiological signal can be identified, based on the determination of the predominant frequency components. At 1010, at least one physiological signal is generated, such as a heart rate signal, a respiration signal, or a Mayer wave signal. These signals each can be associated with one or more corresponding dominant frequency component that are used to form the one or more physiological signals.

FIG. 11 is a block diagram depicting an example of a physiological signal extractor, according to some embodiments. Diagram 1100 depicts a physiological signal extractor 1136 that includes a stream selector 1140, a data correlator 1142, an optional window validator 1143, a parameter estimator 1144, and a separation filter 1146. Physiological signal extractor 1136 can also include an optional offset generator 1139 to be discussed later. As shown in FIG. 11, physiological signal extractor 1136 receives a raw sensor signal from, for example, a bioimpedance sensor, and also receives one or more motion sensor signals 1143 from a motion sensor 1141, which can include one or more accelerometers in some examples. Multiple data streams can represent accelerometer data in multiple axes. Stream selector 1140 is configured to receive, for example, multiple accelerometer signals specifying motion along one or more different axes. Further, stream selector 1140 is configured to select an accelerometer data stream having a greatest motion component (e.g., the greatest magnitude of acceleration for an axis). In some examples, stream selector 1140 is configured to select the axis of acceleration having the highest variability in motion, whereby that axis can be used to track motion or identify a general direction or plane of motion. Optionally, offset generator 1139 can receive a magnitude of the raw sensor signal to modify the dynamic range of an amplifier receiving the raw sensor signal prior to that signal entering data correlator 1142.

Data correlator 1142 is configured to receive the raw sensor signal and the selected stream of accelerometer data. Data correlator 1142 operates to correlate the sensor signal and the selected motion sensor signal. For example, data correlator 1142 can scale the magnitudes of the selected motion sensor signal to an equivalent range for the sensor signal. In some embodiments, data correlator 1142 can provide for the transformation of the signal data between the bioimpedance sensor signal space and the acceleration data space. Such a transformation can be optionally performed to make the motion sensor signals, especially the selected motion sensor signal, equivalent to the bioimpedance sensor signal. In some examples, a cross-correlation function or an autocorrelation function can be implemented to correlate the sets of data representing the motion sensor signal and the sensor signal.

Parameter estimator 1144 is configured to receive the selected motion sensor signal from stream selector 1140 and the correlated data signal from data correlator 1142. In some examples, parameter estimator 1144 is configured to estimate parameters, such as coefficients, for filtering out physiological characteristic signals from motion-related artifact signals. For example, the selected motion sensor signal, such as accelerometer signal, generally does not include biological derived signal data, and, as such, one or more coefficients for physiological signal components can be reduced or effectively determined to be zero. Separation filter 1146 is configured to receive the coefficients as well as data correlated by data correlator 1142 and the selected motion sensor signal from stream selector 1140. In operation, separation filter 1146 is configured to recover the sources of the signals. For example, separation filter 1146 can generate a recovered physiological characteristic signal (“P”) 1160 and a recovered motion signal (“M”) 1162. Separation filter 1146, therefore, operates to separate a sensor signal including both biological signals and motion-related artifact signals into additive or subtractable components. Recovered signals 1160 and 1162 can be used to further determine one or more physiological characteristics signals, such as a heart rate, respiration rate, and a Mayer wave.

Window validator 1143 is optional, according to some embodiments. Window validator 1143 is configured to receive motion sensor signal data to determine a duration time (i.e., a valid window of time) in which sensor signal data can be predicted to be valid (i.e., durations in which the magnitude of motion-related artifacts signals likely do not affect the physiological signals). In some cases, window validator 1143 is configured to predict a saturation condition for a front-end amplifier (or any other condition, such as a motion-induced condition), whereby the sensor signal data is deemed invalid.

FIG. 12 depicts an example of an offset generator according to some embodiments. Diagram 1200 depicts offset generator 1239 including a dynamic range determinator 1240 and an optional amplifier 1242, which can be disposed within or without offset generator 1239. In sensing bioimpedance-related signals, the bioimpedance signals generally are “small-signal;” that is, these signals have relatively small amplitudes that can be distorted by changes in impedances, such as when the coupling between the electrodes and the skin is disrupted. Offset generator 1239 can be configured to determine an amount of motion that is associated with motion sensor signal (“M”) 1260, such as an accelerometer signal, and to adjust the dynamic range of operation of amplifier 1242, which can be an operational amplifier configured as a front-end amplifier. Further, offset generate 1239 can also be optionally configured to receive sensor signal (“S”) 1262 and correlated data (“CD”) 1264, either or both of which can be used to determine first whether to modify the dynamic range of amplifier 1242, and if so, to what degree to which the dynamic range ought to be modified. In some cases, the degree to which the dynamic range ought to be modified specified by an offset value. As shown, amplifier 1242 is configured to generate an offset sensor signal 1244 that is conditioned or otherwise adapted to avoid or reduce clipping.

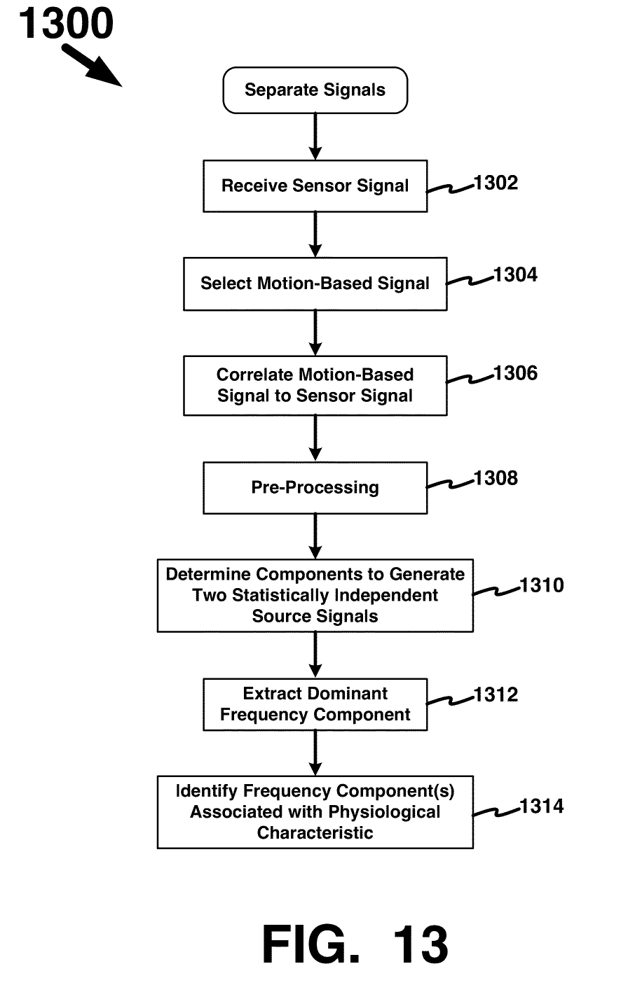

FIG. 13 is a flowchart depicting example of a flow for decomposing a sensor signal to form separate signals, according to some embodiments. Flow 1300 can be implemented in a variety of different ways using a number of different techniques. In some examples, flow 1300 and its elements can be implemented by one or more of the components or elements described herein, according to various embodiments. In the following example, while not intended to be limiting, flow 1300 is described in terms of an analysis for extracting physiological characteristic signals in accordance with one or more techniques of performing Independent Component Analysis (“ICA”). At 1302, a sensor signal is received, and at 1304 a motion sensor signal is selected. When a test subject, or user, is wearing a wearable device and is physically active, the received bioimpedance signal can include two signals: 1.) a sensor signal including one or more physiological signals such as heart rate, respiration rate, and Mayer waves, and 2.) motion-related artifact signals. Further, the one or more physiological signals and motion sensor signals (or motion-related artifact signals) may be correlated at 1306. In this example, a physiological signal is assumed to be statistically independent (or nearly statistically independent) of a motion sensor signal or related artifacts. In some examples, flow 1300 provides for separating a multivariate signal into additive or subtractive subcomponents, based on a presumed mutually-statistical independence between non-Gaussian source signals. Statistical independence of estimated physiological sample components and motion related artifact signal components can be maximized based on for example minimizing mutual information, and maximizing non-Gaussianity of the source signals.

Further to flow 1300, consider two statistically independent noun Gaussian source signals S1 and S2, and two observation points O1 and O2. In some examples, observation points O1(t) and O2(t) are time-indexed samples associated with observed samples from the same sensor, at different locations. For example, O1(t) and O2(t) can represent observed samples from a first bioimpedance sensor (or electrode) and from a second bioimpedance sensor (or electrode), respectively. In other examples, O1(t) and O2(t) can represent observed samples from a first sensor, such as a bioimpedance sensor, and a second sensor, such as an accelerometer, respectively. At 1308, data associated with one or more of the two observation points O1 and O2 are preprocessed. For example, the data for the observation points can be centered, whitened, and/or reduced in dimensions, wherein preprocessing may reduce the complexity of determining the source signals and/or reduce the number of parameters or coefficients to be estimated. An example of a centering process includes subtracting the meaning of data from a sample to translate samples about a center. An example of a whitening process is eigenvalue decomposition. In some embodiments, preprocessing at 1308 can be different from, or similar to, the correlation of data as described herein, at least in some cases.

Observation points O1(t) and O2(t) can be expressed as follows:

O₁(t)=a₁₁S1+a₁₁S2 (Eqn. 1)

O₂(t)=a₂₁S1+a₂₁S2 (Eqn. 2)

where O=A×S, which represent matrices, and a11, a12, a21, and a22 represent parameters (or coefficients) that can be estimated. At 1310, the above equations 1 and 2 can be used to determine components for generating two (2) statistically-independent source signals, whereby A and S can be extracted from O. In some examples, A and S can be extracted iteratively, based on user-specified error rate and/or maximum number of iterations, among other things. Further, coefficients a11, a12, a21, and a22 can be modified such that one or more coefficients for the physiological characteristic and biological component is set to or near zero, as the accelerometer signal generally does not include physiological signals. In at least one embodiment, parameter estimator 1144 of FIG. 11 can be configured to determine estimated coefficients.

In some examples a matrix can be formed based on estimated coefficients, at 1310. At least some of the coefficients are configured to attenuate values of the physiological signal components for the motion sensor signal. An example of the matrix is a mixing matrix. Further, the matrix of coefficients can be inverted to form an inverted mixing matrix (e.g., to form an “unmixing” matrix). The inverted mixing matrix of coefficients can be applied (e.g., iteratively) to the samples of observation points O1(t) and O2(t) to recover the source signals, such as a recovered physiological characteristic signal and a recovered motion signal (e.g. a recovered motion-related artifact signal). In at least one embodiment, separation filter 1146 of FIG. 11 can be configured to apply an inverted matrix to samples of the physiological signal components and the motion signal components to determine the recovered physiological characteristic signal and the recovered motion signal (e.g., a recovered muscle movement signal). Note that various described functionalities of flow 1300 can be implemented in or distributed over one or more of the described structures set forth herein. Note, too, that while flow 1300 is described in terms of ICA in the above-mentioned examples, flow 1300 can be implemented using various techniques and structures, and the various embodiments are neither restricted nor limited to the use of ICA. Other signal separation processes may also be implemented, according to various embodiments.

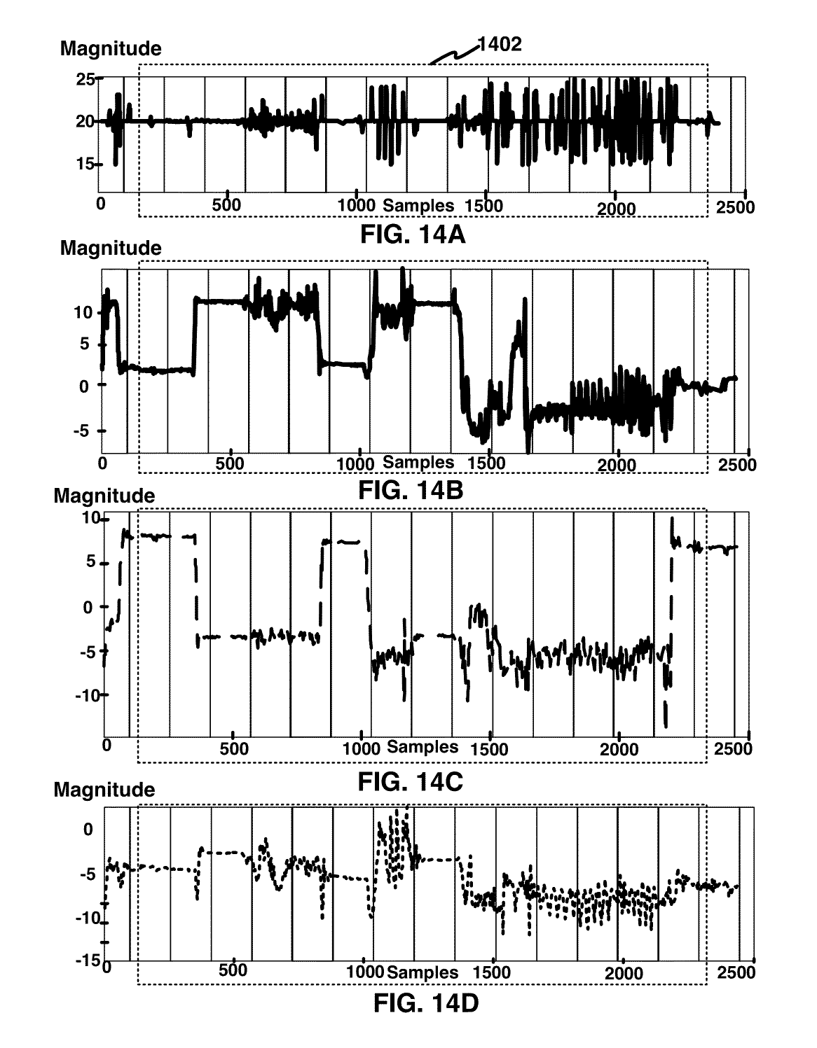

FIGS. 14A to 14D depict various signals used for physiological characteristic signal extraction, according to various embodiments. FIG. 14A depicts a sensor signal received as, for example, a bioimpedance signal in which the magnitude varies about 20 over a number of samples. In this example, validation window can be used for heart rate extraction, whereby the sensor signal is down-sampled by, for example, a factor of 100 (i.e., the sensor signal is sampled at, for example, 15.63 Hz). Also shown in FIG. 14A is an optional window 1402 that indicates a validation window in which data is deemed valid as determined by, for example, window validator 1143 of FIG. 11. Returning back to FIGS. 14A to 14C, FIG. 14B depicts a first stream of accelerometer data for a first axis. FIG. 14C and FIG. 14D depict a second stream of accelerometer data for a second axis and a third stream of accelerometer data for a third axis, respectively. FIGS. 14A to 14C are intended to depict only a few of many examples and implementations.

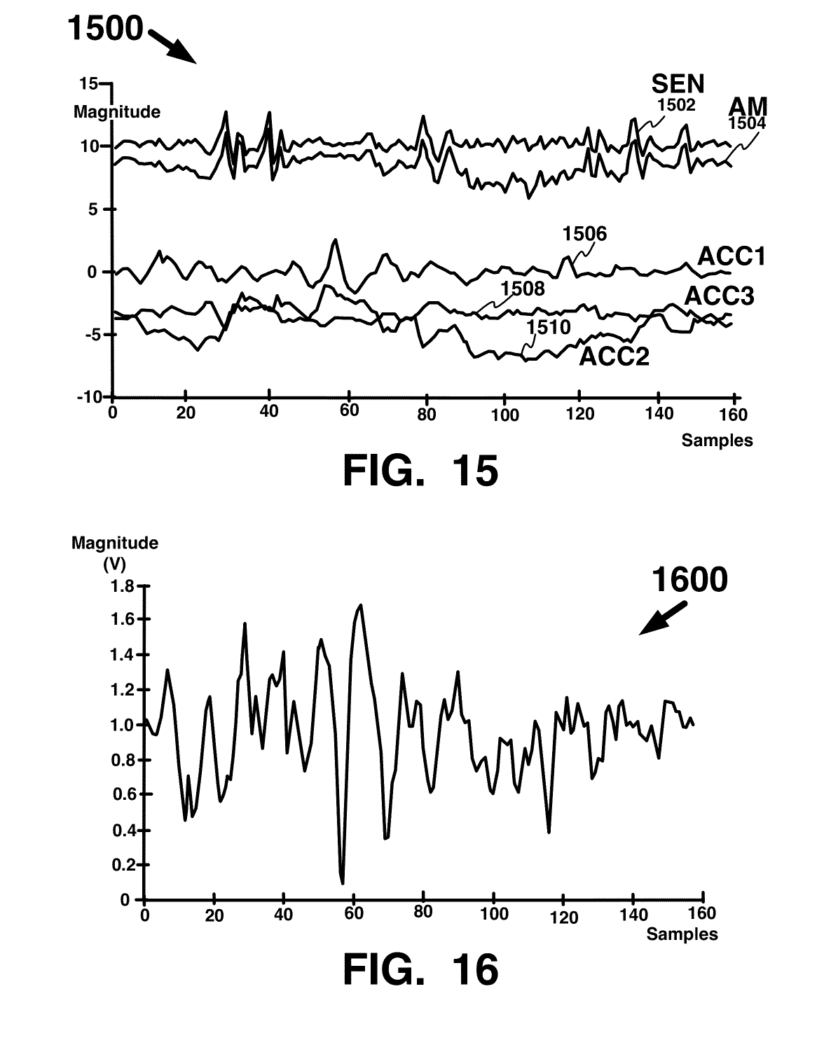

FIG. 15 depicts recovered signals, according to some embodiments. Diagram 1500 depicts the magnitudes of various signals over 160 samples. Signal 1502 represents us magnitude of the sensor signal, whereas signal 1504 represents the magnitude of an accelerometer signal. Signals 1506, 1508, and 1510 represent the magnitudes of a first of accelerometer signal, a second accelerometer signal, and a third accelerometer signal, respectively.

FIG. 16 depicts an extracted physiological signal, according to various embodiments. Diagram 1600 depicts the magnitude, in volts, of an extracted physiological characteristic signal using the first accelerometer stream as the selected accelerometer stream. For this example, a fast Fourier transform (“FFT”) analysis of the data set forth in FIG. 16 yields a heart rate estimated at, for example, 77.6274 bpm.

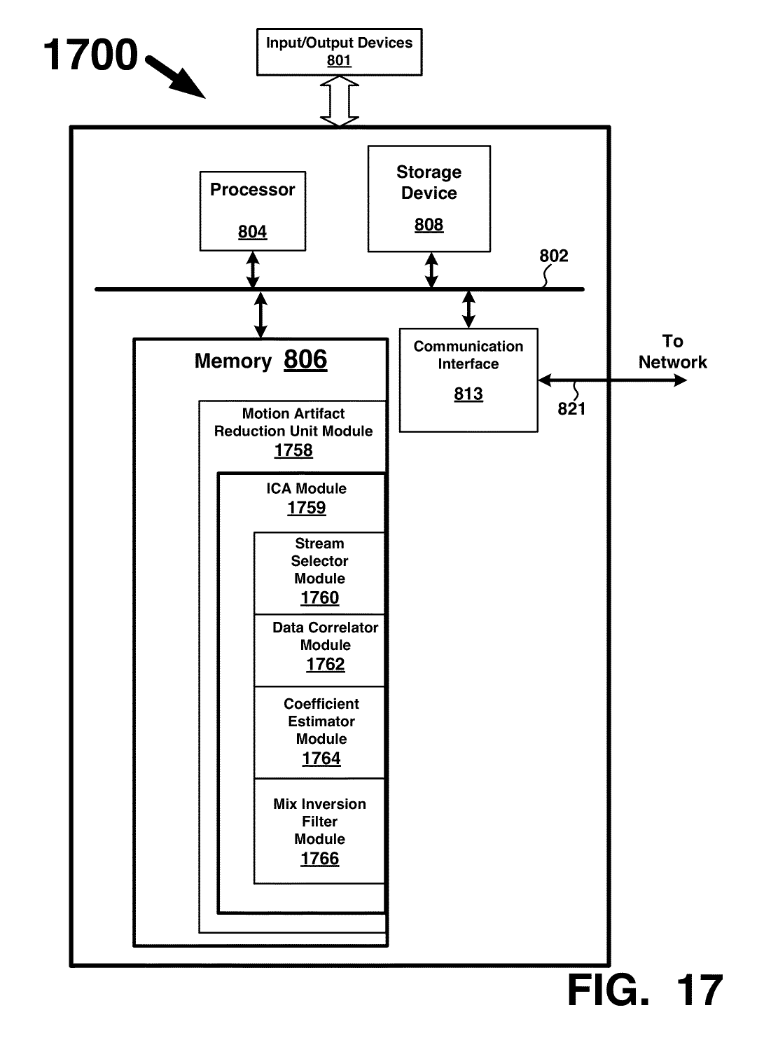

FIG. 17 illustrates an exemplary computing platform disposed in a wearable device in accordance with various embodiments. In some examples, computing platform 1700 may be used to implement computer programs, applications, methods, processes, algorithms, or other software to perform the above-described techniques, and can include similar structures and/or functions as set forth in FIG. 8. But in the example shown, system memory 806 can include various modules that include executable instructions to implement functionalities described herein. In the example shown, system memory 806 includes a motion artifact reduction unit module 1758 configured to determine physiological information relating to a user that is wearing a wearable device. Motion artifact reduction unit module 1758 can include a stream selector module 1760, a data correlator module 1762, a coefficient estimator module 1764, and a mix inversion filter module 1766, any of which can be configured to provide one or more functions described herein.

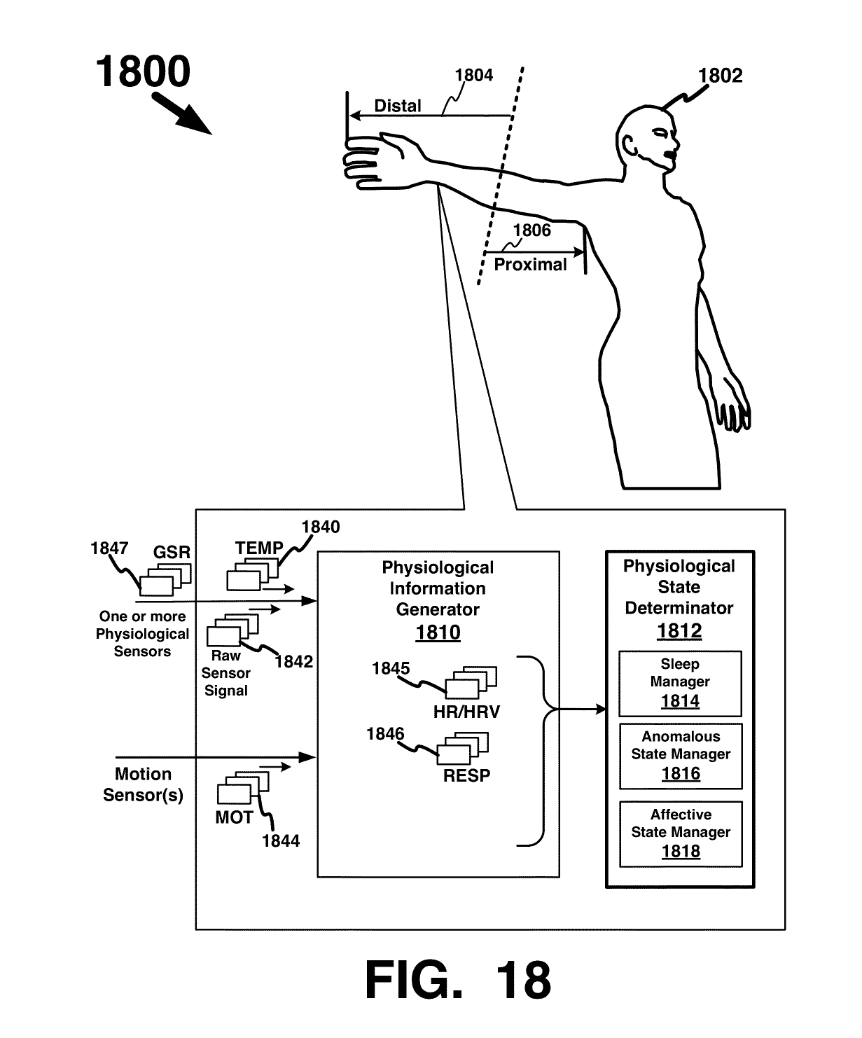

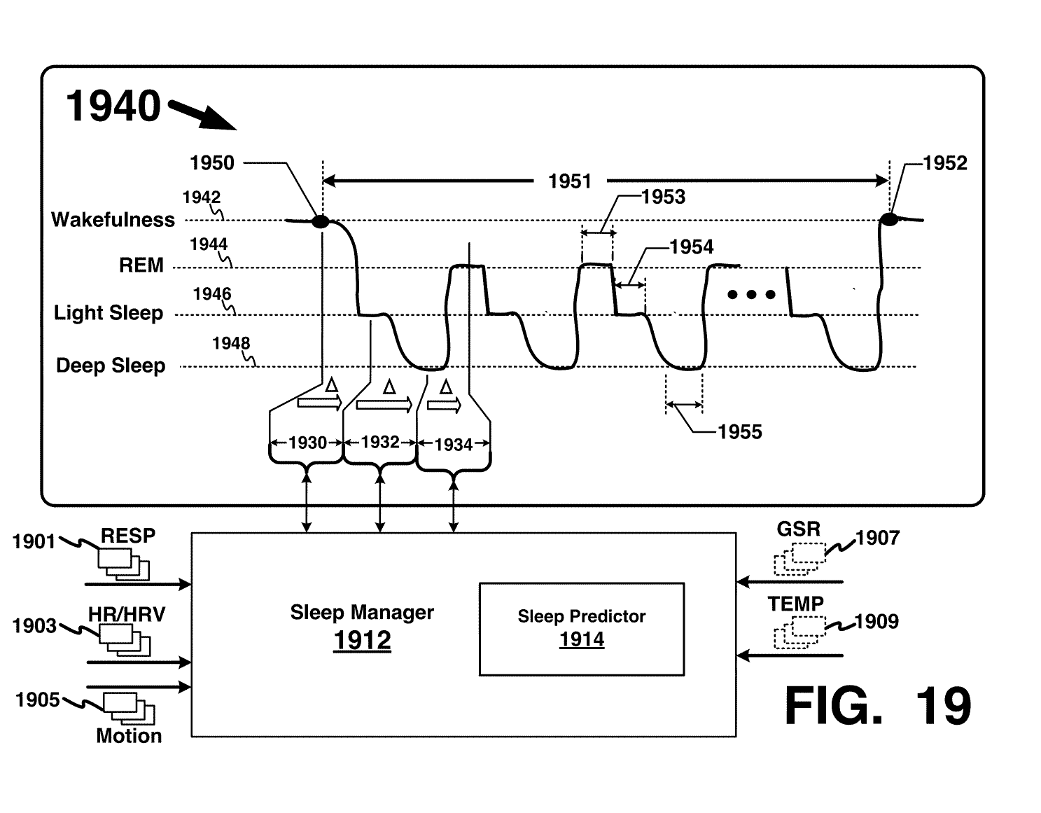

FIG. 18 is a diagram depicting a physiological state determinator configured to receive sensor data originating, for example, at a distal portion of a limb, according to some embodiments. As shown, diagram 1800 depicts a physiological information generator 1810 and a physiological state determinator 1812, which, at least in the example shown, are configured to be disposed at, or receive signals from, at a distal portion 1804 of a user 1802. In some embodiments, physiological information generating 1810 and physiological state determinator 1812 are disposed in a wearable device (not shown). Physiological information generator 1810 configured to receive signals and/or data from one or more physiological sensors and one or more motion sensors, among other types of sensors. In the example shown, physiological information generator 1810 is configured to receive a raw sensor signal 1842, which can be similar or substantially similar to other raw sensor signals described herein. Physiological information generator 1810 is also configured to receive other sensor signals including temperature (“TEMP”) 1840, skin conductance (depicted as GSR data signal 1847), pulse waves, heat rates (e.g., heart beats-per-minute), respiration rates, heart rate variability, and any other sensed signal configured to include physiological information or any other information relating to the physiology of a person. Examples of other sensors are described in U.S. patent application Ser. No. 13/454,040, filed on Apr. 23, 2012, which is incorporated by reference. Physiological information generator 1810 is also configured to receive motion (“MOT”) signal data 1844 from one or more motion sensor(s), such as accelerometers. Note that raw sensor signal 1842 can be an electrical signal, such as a bioimpedance signal, or an acoustic signal, or any other type of signal. According to some embodiments, physiological information generator 1810 is configured to extract physiological signals from a raw sensor signal 1842. For example, a heart rate (“HR”) signal and/or heart rate variability (“HRV”) signal 1845 and respiration rate (“RESP”) 1846 can be determined for example, by a motion artifact reduction unit (not shown). Physiological information generator 1810 is configured to convey sensed physiological characteristics signals or derive physiological characteristic signals (e.g., from sensed signals) for use by physiological state determinator 1812. In some examples, a physiological characteristic signal can include electrical impulses of muscles (e.g., as evidenced, in some cases, by electromyography (“EMG”) to determine the existence and/or amounts of motion based on electrical signals generated by muscle cells at rest or in contraction.

As shown, physiological state determinator 1812 includes a sleep manager 1814, an anomalous state manager 1816, and an affective state manager 1818. Physiological state determinator 1812 is configured to receive various physiological characteristics signals and to determine a physiological state of a user, such as user 1802. Physiological states include, but are not limited to, states of sleep, wakefulness, a deviation from a normative physiological state (i.e., an anomalous state), an affective state (i.e., mood, feeling, emotion, etc.). Sleep manager 1814 is configured to detect a stage of sleep as a physiological state, the stages of sleep including REM sleep and non-REM sleep, including as light sleep and deep sleep. Sleep manager 1814 is also configured to predict the onset or change into or between different stages of sleep, even if such changes are imperceptible to user 1802. Sleep manager 1814 can detect that user 1802 is transitioning from a wakefulness state to a sleep state and, for example, can generate a vibratory response (i.e., generated by vibration) or any other alert to user 1802. Sleep manager 1814 also can predict a sleep stage transition to either alert user 1802 or to disable such an alert if, for example, the alert is an alarm (i.e., wake-up time alarm) that coincides with a state of REM sleep. By delaying generation of an alarm, the user 1802 is permitted to complete of a state of REM sleep to ensure or enhance the quality of sleep. Such an alert can assist user 1802 to avoid entering a sleep state from a wakefulness state during critical activities, such as driving.

Anomalous state manager 1860 is configured to detect a deviation from the normative general physiological state in reaction, for example, to various stimuli, such as stressful situations, injuries, ailments, conditions, maladies, manifestations of an illness, and the like. Anomalous state manager 1860 can be configured to determine the presence of a tremor that, for example, can be a manifestation of an ailment or malady. Such a tremor can be indicative of a diabetic tremor, an epileptic tremor, a tremor due to Parkinson's disease, or the like. In some embodiments, anomalous state manager 1860 is configured to detect the onset of tremor related to a malady or condition prior to user 1802 perceiving or otherwise being aware of such a tremor. Therefore, anomalous state manager 1860 can predict the onset of a condition that may be remedied by, for example, medication and can alert user 1802 to the impending tremor. User 1802 then can take the medication before the intensity of the tremor increases (e.g., to an intensity that might impair or otherwise incapacitate user 1802). Further, anomalous state manager 1860 can be configured to determine if the physiological state of user 1802 is a pain state, in which user 1802 is experiencing pain. Upon determining a pain state, a wearable device (not shown) can be configured to transmit the presence of pain to a third-party via a wireless communication path to alert others of the pain state for resolution.

Affective state manager 1818 is configured to use at least physiological sensor data to form affective state data representing an approximate affective state of user 1802. As used herein, the term “affective state” can refer, at least in some embodiments, to a feeling, a mood, and/or an emotional state of a user. In some cases, affective state data can includes data that predicts an emotion of user 1802 or an estimated or approximated emotion or feeling of user 1802 concurrent with and/or in response to the interaction with another person, environmental factors, situational factors, and the like. In some embodiments, affective state manager 1818 is configured to determine a level of intensity based on sensor derived values and to determine whether the level of intensity is associated with a negative affectivity (e.g., a bad mood) or positive affectivity (e.g., a good mood). An example of an affective state manager 1818 is an affective state prediction unit as described in U.S. Provisional Patent Application No. 61/705,598 filed on Sep. 25, 2012, which is incorporated by reference herein for all purposes. While affective state manager 1818 is configured to receive any number of physiological characteristics signals in which to determine of an affective state of user 1802, affective state manager 1818 can use sensed and/or derived Mayer waves based on raw sensor signal 1842. In some examples, the detected Mayer waves can be used to determine heart rate variability (“HRV”) as heart rate variability can be correlated to Mayer waves. Further, affective state manager 1818 can use, at least in some embodiments, HRV to determine an affective state or emotional state of user 1802 as HRV may correlate with an emotion state of user 1802. Note that, while physiological information generating 1810 and physiological state determinator 1812 are described above in reference to distal portion 1804, one or more of these elements can be disposed at, or receive signals from, proximal portion 1806, according to some embodiments.

FIG. 19 depicts a sleep manager, according to some embodiments. As shown, FIG. 19 depicts a sleep manager 912 including a sleep predictor 1914. Sleep manager 1912 is configured to determine physiological states of sleep, such as a sleep state or a wakefulness state in which the user is awake. Sleep manager 1912 is configured to receive physiological characteristic signals, such as data representing respiration rates (“RESP”) 1901, heart rate (“HR”) 1903 (or heart rate variability, HRV), motion-related data 1905, and other physiological data such as optional skin conductance (“GSR”) 1907 and optional temperature (“TEMP”) 1909, among others. As shown in diagram 1940, a person who is sleeping passes through one or more sleep cycles over a duration 1951 between a sleep start time 1950 and sleep end time 1952. There is a general reduction of motion when a person passes from a wakefulness state 1942 into the stages of sleep, such as into light sleep 1946 in duration 1954. Motion indicative of “hypnic jerks” or involuntary muscle twitching motions typically occur during light sleep state 1946. The person then passes into a deep sleep state 1948, in which, a person has a decreased heart rate and body temperature, with the absence of voluntary muscle motions to confirm or establish that a user is in a deep sleep state. Collectively, the light sleep state and the deep sleep state can be described as non-REM sleep states. Further to diagram 1940, the sleeping person then passes into an REM sleep state 1944 for duration 1953 during which muscles can be immobile.

According to some embodiments, sleep manager 1912 is configured to determine a stage of sleep based on at least the heart rate and respiration rate. For example, sleep manager 1912 can determine the regularity of the heart rate and respiration rate to determine the person is in a non-REM sleep state, and, thereby, can generate a signal indicating the stage of the sleep is a non-REM sleep states, such as light sleep or deep sleep states. During light sleep and deep sleep, a heart rate and/or the respiration rate of the user can be described as regular or without significant variability. Thus, the regularity of the heart rate and/or respiration rate can be used to determine physiological sleep state of the user. In some examples the regularity of the heart rate and/or the respiration rate can include any heart rate or respiration rate that varies by no more than 5%. In some other cases, the regularity of the heart rate and/or the respiration rate can vary by any amount up to 15%. These percentages are merely examples and are not intended to be limiting, and ordinarily skilled artisan will appreciate that the tolerances for regular heart rates and respiration rates may be based on user characteristics, such as age, level of fitness, gender and the like. Sleep manager 1912 can use motion data 1905 to confirm whether a user is in a light sleep state or a deep sleep state by detecting indicative amounts of motion, such as a portion of motion that is indicative of involuntary muscle twitching.

As another example, sleep manager 1912 can determine the irregularity (or variability) of the heart rate and respiration rate to determine the person is in an REM sleep state, and, thereby, can generate a signal indicating the stage of the sleep is an REM sleep states. During REM sleep, a heart rate and/or the respiration rate of the user can be described as irregular or with sufficient variability to identify that a user is REM sleep. Thus, the variability of the heart rate and/or respiration rate can be used to determine physiological sleep state of the user. In some examples the irregularity of the heart rate and/or the respiration rate can include any heart rate or respiration rate that varies by more than 5%. In some other cases, the variability of the heart rate and/or the respiration rate can vary by any amounts up from 10% to 15%. These percentages are merely examples and are not intended to be limiting, and ordinarily skilled artisan will appreciate that the tolerances for variable heart rates and respiration rates may be based on user characteristics, such as age, level fitness, gender and the like. Sleep manager 1912 can use motion data 1905 to confirm whether a user is in an REM sleep state by detecting indicative amounts of motion, such as a portion of motion that includes negligible to no motion.

Sleep manager 1912 is shown to include sleep predictor 1914, which is configured to predict the onset or change into or between different stages of sleep. The user may not perceive such changes between sleep states, such as transitioning from a wakefulness state to a sleep state. Sleep predictor 1914 can detect this transition from a wakefulness state to a sleep state, as depicted as transition 1930. Transition 1930 may be determined by sleep predictor 1940 based on the transitions from irregular heart rate and respiration rates during wakefulness to more regular heart rates and respiration rates during early sleep stages. Also, lowered amounts of motion can also indicate transition 1930. In some embodiments, motion data 1905 includes a velocity or rate of speed at which a user is traveling, such as an automobile. Upon detecting an impending transition from a wakefulness state into a sleep state, sleep predictor 1914 generates an alert signal, such as a vibratory initiation signal, configuring to generate a vibration (or any other response) to convey to a user that he or she is about to fall asleep. So if the user is driving, predictor 914 assists in maintaining a wakefulness state during which the user can avoid falling asleep behind the wheel. Sleep predictor 1914 can be configured to also detect transition 1932 from a light sleep state to a deep sleep state and a transition 1934 from a deep sleep state to an REM sleep state. In some embodiments, transitions 1932 in 1934 can be determined by detected changes from regular to variable heart rates or respiration rates, in the case of transition 1934. Also, transition 1934 can be described by a decreased level of motion to about zero during the REM sleep state. Further, sleep predictor 1914 can be configured to predict a sleep stage transition to disable an alert, such as wake-up time alarm, that coincides with a state of REM sleep. By delaying generation of an alarm, the user is permitted to complete of a state of REM sleep to enhance the quality of sleep.

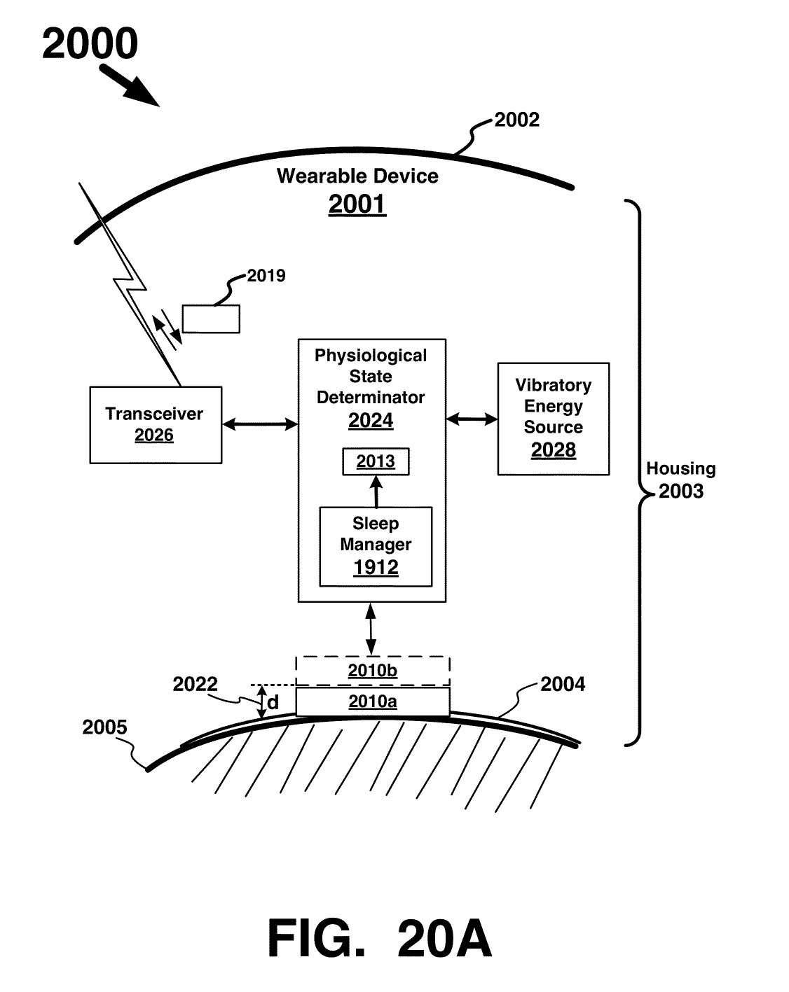

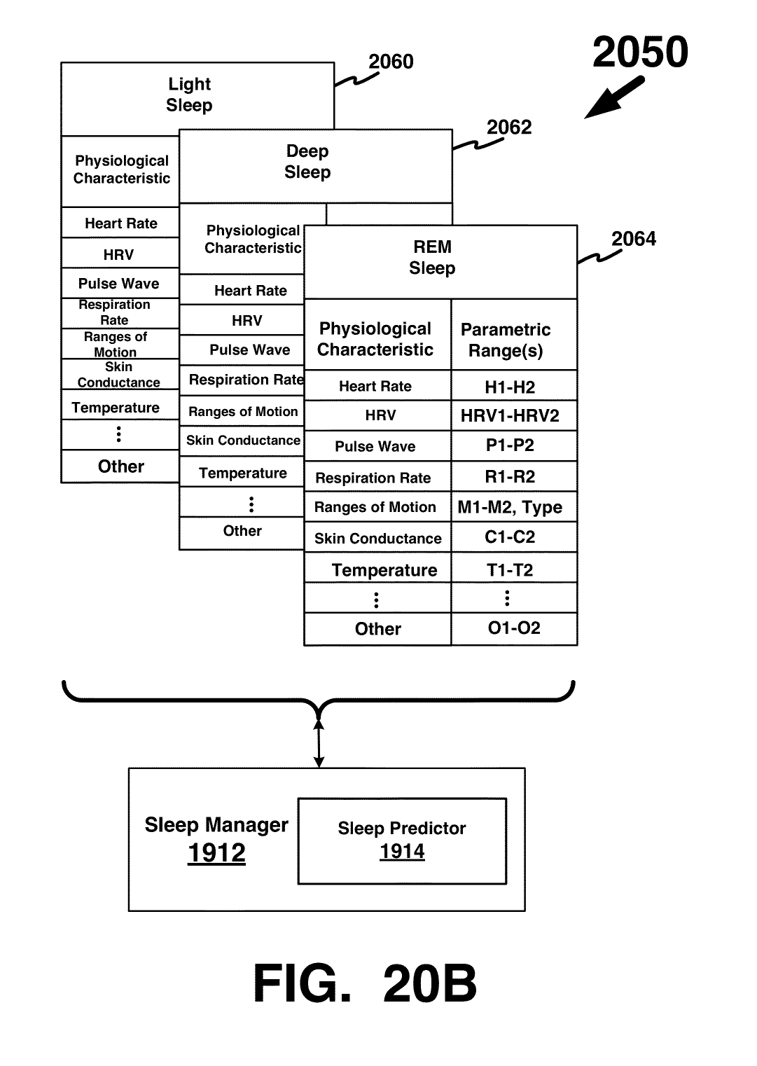

FIG. 20A depicts a wearable device including a skin surface microphone (“SSM”), in various configurations, according to some embodiments. According to various embodiments, a skin surface microphone (“SSM”) can be implemented in cooperation with (or along with) one or more electrodes for bioimpedance sensors, as described herein. In some cases, a skin surface microphone (“SSM”) can be implemented in lieu of electrodes for bioimpedance sensors. Diagram 2000 of FIG. 20 depicts a wearable device 2001, which has an outer surface 2002 and an inner surface 2004. In some embodiments, wearable device 2001 includes a housing 2003 configured to position a sensor 2010a (e.g., an SSM including, for instance, a piezoelectric sensor or any other suitable sensor) to receive an acoustic signal originating from human tissue, such as skin surface 2005. As shown, at least a portion of sensor 2010a can be formed external to surface 2004 of wearable housing 2003. The exposed portion of the sensor can be configured to contact skin 2005. In some embodiments, the sensor (e.g., SSM) can be disposed at position 2010b at a distance (“d”) 2022 from inner surface 2004. Material, such as an encapsulant, can be used to form wearable housing 2003 to reduce or eliminate exposure to elements in the environment external to wearable device 2001. In some embodiments, a portion of an encapsulant or any other material can be disposed or otherwise formed at region 2010a to facilitate propagation of an acoustic signal to the piezoelectric sensor. The material and/or encapsulant can have an acoustic impedance value that matches or substantially matches the acoustic impedance of human tissue and/or skin. Values of acoustic impedance of the material and/or encapsulant can be described as being substantially similar to the human tissue and/or skin when the acoustic impedance of the material and/or encapsulant varies no more than 60% of that of human tissue or skin, according to some examples.

Examples of materials having acoustic impedances matching or substantially matching the impedance of human tissue can have acoustic impedance values in a range that includes 1.5×106 Pa×s/m (e.g., an approximate acoustic impedance of skin). In some examples, materials having acoustic impedances matching or substantially matching the impedance of human tissue can provide for a range between 1.0×106 Pa×s/m and 1.0×107 Pa×s/m. Note that other values of acoustic impedance can be implemented to form one or portions of housing 2003. In some examples, the material and/or encapsulant can be formed to include at least one of silicone gel, dielectric gel, thermoplastic elastomers (TPE), and rubber compounds, but is not so limited. As an example, the housing can be formed using Kraiburg TPE products. As another example, housing can be formed using Sylgard® Silicone products. Other materials can also be used. In some embodiments, sleep manager 1912 detects increase perspiration via skin conductance during an REM sleep state and determines the user is dreaming, whereby in generates a signal to store such an event or generate an other action.

Further to FIG. 20A, wearable device 2001 also includes a physiological state determinator 2024, a sleep manager 1912, a vibratory energy source 2028, and a transceiver 2026. Physiological state determinator 2024 can be configured to receive signals originating as acoustic signals either from sensor 2010a or a sensor at location 2010b via acoustic impedance-matched material. Upon detecting a sleep state condition (e.g., a sleep state transition), sleep manager 1912 can be configured to communicate the condition to physiological state determinator 2024, which, in turn, generates a notification signal as a vibratory activation signal, thereby causing vibratory energy source 2028 (e.g., mechanical motor as a vibrator) to impart vibration through housing 2003 unto a user, responsive to the vibratory activation signal, to indicate the presence of the sleep-related condition (e.g., transitioning from a wakefulness state to a sleep state). According to some embodiments, sleep manager 1912 can generate a wake enable/disable signal 2013 configured to enable or disable the ability of vibratory energy source 2028 to generate an alarm signal. For example, if sleep manager 1912 determines that the user is in a REM sleep state, sleep manager 1912 generates a wake disable signal 2013 to prevent vibratory energy source 2228 from waking the user. But if sleep manager 1912 determines that the user is in a non-REM sleep state that coincides with a wake alarm time, or is there shortly thereafter, sleep manager 1912 will generate enable signal 2013 to permit vibratory energy source 2028 to wake up the user. In some cases, a wake enable signal and awake disable signal can be the same signal, but at different states. Also, wearable device 2001 can optionally include a transceiver 2026 configured to transmit signal 2019 as a notification signal via, for example, an RF communication signal path. In some examples, transceiver 2026 can be configured to transmit signal 2019 to include data representative of the acoustic signal received from sensor 2010, such as an SSM.

FIG. 20B depicts an example of physiological characteristics and parametric values that can identify a sleep state, according to some embodiments. Diagram 2050 depicts a data arrangement 2060 including data for determining light sleep states, a data arrangement 2062 that includes data for determining deep sleep states, and data arrangement 2064 that includes data for determining REM sleep states, according to various embodiments. Also shown in FIG. 20B, sleep manager 1912 and sleep predictor 1914 can use data arrangements 2060, 2062 and 2064 to determine the various sleep stages of the user. As shown generally, each of the sleep states can be defined one or more physiological characteristics, such as heart rate, HRV, pulse wave, respiration rate, ranges of motion, types of motion, skin conductance, temperature, and any other physiological characteristic or information. As shown, each physiological characteristic is associated with a parametric range that may include one or more than one value associated with the physical physiological characteristic. For example, should the heart rate of a user fall within the range H1-H2, as shown in data arrangement 2064, sleep manager can use this information in determining whether the user is in REM sleep. In some cases, the parametric values that set forth the ranges, maybe based on characteristics of a user, such as age, level of fitness, gender, etc. In one example, sleep manager 1912 operates to analyze the various values of the physiological characteristics and calculates a best-fit determination of the parametric values to identify the corresponding sleep state for the user. The physiological characteristics and parametric values, and data arrangements 2062 to 2064 is merely one example and is not intended to be limiting.

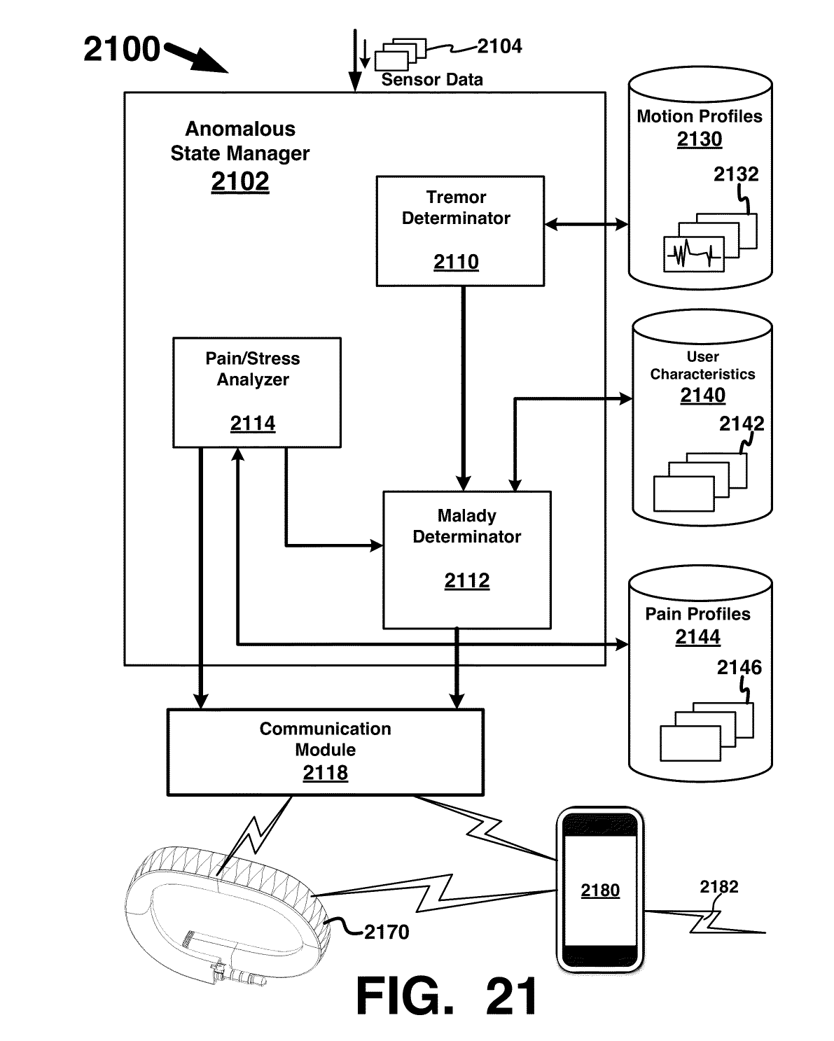

FIG. 21 depicts an anomalous state manager 2102, according to some embodiments. Diagram 2100 depicts that anomalous state manager 2102 includes a tremor determinator 2110, a pain/stress analyzer 2114 and a malady determinator 2112. Anomalous state manager 2102 receives sensor data 2104 and is configured to detect a deviation from the normative general physiological state of a user responsive, for example, to various stimuli, such as stressful situations, injuries, ailments, conditions, maladies, manifestations of an illness, symptoms of a condition, and the like. Also shown in diagram 2100 are repositories accessible by anomalous state manager 2102, including motion profile repository 2130, user characteristic repository 2140 and pain profile repository 2144. Motion profile repository 2130 includes profile data 2132 that includes data defining configured to define a tremor, or a portion thereof, associated with detected motion. User characteristic repository 2140 includes user-related data 2142 that describes the user, for example, in terms of age, fitness level, gender, diseases, conditions, ailments, maladies, and any other characteristic that may influence the determination of the physiological state of the user. Pain profiles 2144 includes data 2146 that can define whether the user is in a pain state. In some embodiments, data 2146 is a data arrangement that includes physiological characteristics similar to those shown in FIG. 20B. For example, physiological signs of pain may include, for example, an increase in respiration rate, an increase in the length of a respiration cycle (e.g., deeper inhalation and exhalation), changes and/or variations in blood pressure, changes and/or variations in heart rate, an increase in perspiration (e.g., increased skin conductance), an increase in muscle tone (e.g., as determined by physiological characteristics indicating increased electrical impulses to or by musculature, and the like). Based on such physiological characteristics, pain/stress analyzer 2114 can be configured to detect that the user is experiencing pain, and in some cases, the level of pain. Further, pain/stress analyzer 2114 can be configured to transmit data representing pain state information to a communication module 2118 for transmitting of the pain state-related information via wearable device 2170 or other mobile devices 2180 to a third-party (or any other entity or computing device) via communications path 2182 (e.g., wireless communications path and/or networks).

Tremor determinator 2110 is configured to determine the presence of a tremor that, for example, can be a manifestation of an ailment or malady. As discussed, such a tremor can be indicative of a diabetic tremor, an epileptic tremor, a tremor due to Parkinson's disease, or the like. In some embodiments, tremor determinator 2110 is configured to detect the onset of tremor related to a malady or condition prior to a user perceiving or otherwise being aware of such a tremor. In particular, wearable devices disposed at a distal portion of a limb may be more likely, at least in some cases, to detect tremors more readily than when disposed at a proximal portion.

Therefore, anomalous state manager 2102 can predict the onset of a condition that may be remedied by, for example, medication and can alert a user to the impending tremor. In some cases, malady determinator 2112 is configured to receive data representing a tremor and data 2142 representing user characteristics, and is further configured to determine the malady afflicting the user. For example, if data 2142 indicates the user is a diabetic, the tremor data received from tremor determinator 2110 is likely to indicate a diabetic-related tremor. Therefore, malady determinator 2112 can be configured to generate an alert that, for example, the user's blood glucose is decreasing to low level amounts that cause such diabetic tremors. The alert can be configured to prompt the user to obtaining medication to treat the impending anomalous physiological state of the user. In another example, tremor determinator 2110 in malady determinator 2112 cooperate to determine that the user is experiencing and an epileptic tremor, and generates an alert to enable the user to either take medication or stop engaging in a critical activity, such as driving, before the tremors become worse (i.e., to an intensity that might impair or otherwise incapacitate the user). Upon detection of tremor and the corresponding malady, anomalous state manager 2102 transmits data indicating the presence of such tremors via communication module 2118 to wearable device 2170 or mobile computing device 2180, which, in turn, transmit via networks 2182 to a third-party or any other entity. In some examples, anomalous state manager 2102 is configured to distinguish malady-related tremors from movements and/or shaking due to nervousness and or injury.

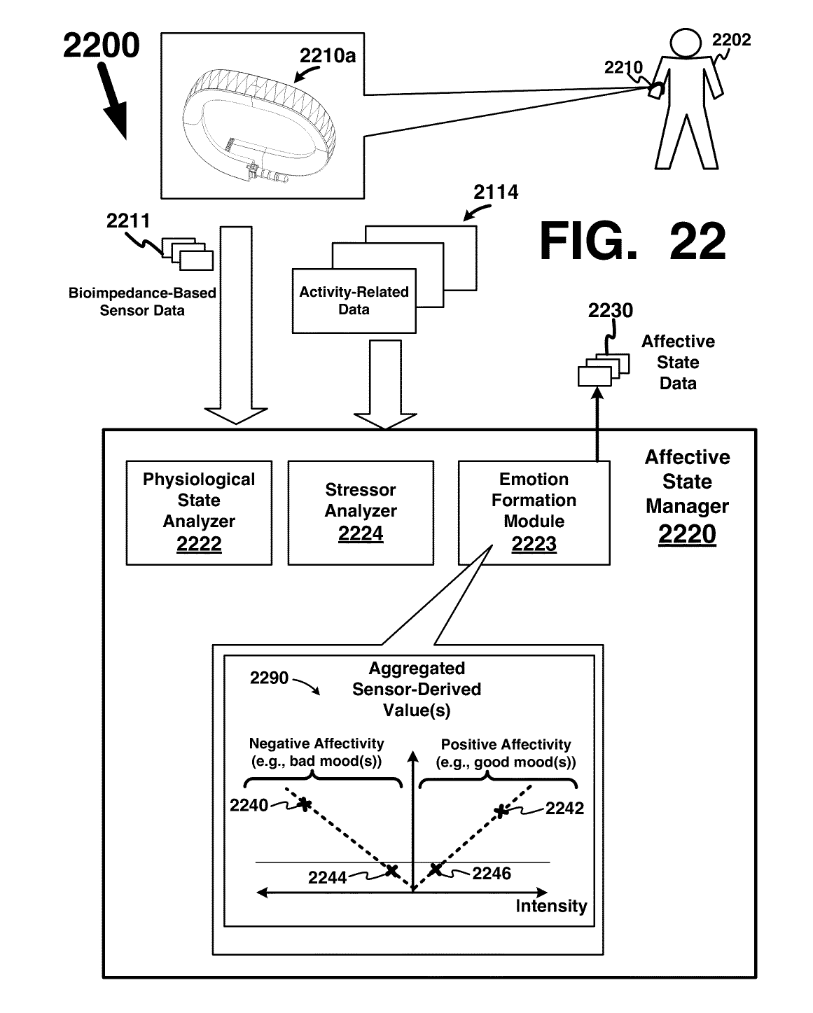

FIG. 22 depicts an affective state manager configured to receive sensor data derived from bioimpedance signals, according to some embodiments. FIG. 22 illustrates an exemplary affective state manager 2220 for assessing affective states of a user based on data derived from, for example, a wearable computing device, according to some embodiments. Diagram 2200 depicts a user 2202 including a wearable device 2210, whereby user 2202 experiences one or more types of stimuli that can changes in physiological states of user 2202, such as the emotional state of mind. In some embodiments, wearable device 2210 is a wearable computing device 2210a that includes one or more sensors to detect attributes of the user, the environment, and other aspects of the responses from/interaction with stimuli.

Affective state manager 2220 is shown to include a physiological state analyzer 2222, a stressor analyzer 2224, and an emotion formation module 2223. According to some embodiments, physiological state analyzer 2222 is configured to receive and analyze the sensor data, such as bioimpedance-based sensor data 2211, to compute a sensor-derived value representative of an intensity of an affective state of user 2202. In some embodiments, the sensor-derived value can represent an aggregated value of sensor data (e.g., an aggregated an aggregated value of sensor data value). In some examples, aggregated value of sensor data can be derived by, first, assigning a weighting to each of the values (e.g., parametric values) sensed by the sensors associated with one or more physiological characteristics, such as those shown in FIG. 20B, and, second, aggregating each of the weightings to form an aggregated value. Affective state manager 2220 can also receive activity-related data 2114 from a number of activity-related managers (not shown). One or more activity-related managers (not shown) can be configured to receive data representing parameters relating to one or more motion or movement-related activities of a user and to maintain data representing one or more activity profiles. Activity-related parameters describe characteristics, factors or attributes of motion or movements in which a user is engaged, and can be established from sensor data or derived based on computations. Examples of parameters include motion actions, such as a step, stride, swim stroke, rowing stroke, bike pedal stroke, and the like, depending on the activity in which a user is participating. As used herein, a motion action is a unit of motion (e.g., a substantially repetitive motion) indicative of either a single activity or a subset of activities and can be detected, for example, with one or more accelerometers and/or logic configured to determine an activity composed of specific motion actions.

According to some examples, the activity-related managers can include a nutrition manager, a sleep manager, an activity manager, a sedentary activity manager, and the like, examples of which can be found in U.S. patent application Ser. No. 13/433,204, filed on Mar. 28, 2012 having Attorney Docket No. ALI-013CIP1; U.S. patent application Ser. No. 13/433,208, filed Mar. 28, 2012 having Attorney Docket No. ALI-013CIP2; U.S. patent application Ser. No. 13/433,208, filed Mar. 28, 2012 having Attorney Docket No. ALI-013CIP3; U.S. patent application Ser. No. 13/454,040, filed Apr. 23, 2012 having Attorney Docket No. ALI-013CIP1CIP1; U.S. patent application Ser. No. 13/627,997, filed Sep. 26, 2012 having Attorney Docket No. ALI-100; all of which are incorporated herein by reference for all purposes.

In some embodiments, stressor analyzer 2224 is configured to receive activity-related data 2114 to determine stress scores that weigh against a positive affective state in favor of a negative affective state. For example, if activity-related data 2114 indicates user 402 has had little sleep, is hungry, and has just traveled a great distance, then user 2202 is predisposed to being irritable or in a negative frame of mine (and thus in a relatively “bad” mood). Also, user 2202 may be predisposed to react negatively to stimuli, especially unwanted or undesired stimuli that can be perceived as stress. Therefore, such activity-related data 2114 can be used to determine whether an intensity derived from physiological state analyzer 2222 is either negative or positive, as shown.

Emotive formation module 2223 is configured to receive data from physiological state analyzer 2222 and stressor analyzer 2224 to predict an emotion in which user 2202 is experiencing (e.g., as a positive or negative affective state). Affective state manager 2220 can transmit affective state data 2230 via network(s) to a third-party, another person (or a computing device thereof), or any other entity, as emotive feedback. Note that in some embodiments, physiological state analyzer 2222 is sufficient to determine affective state data 2230. In other embodiments, stressor analyzer 2224 is sufficient to determine affective state data 2230. In various embodiments, physiological state analyzer 2222 and stressor analyzer 2224 can be used in combination or with other data or functionalities to determine affective state data 2230.

As shown, aggregated sensor-derived values 2290 can be generated by a physiological state analyzer 2222 indicating a level of intensity. Stressor analyzer 2224 is configured to determine whether the level of intensity is within a range of negative affectivity or is within a range of positive affectivity. For example, an intensity 2240 in a range of negative affectivity can represent an emotional state similar to, or approximating, distress, whereas intensity 2242 in a range of positive affectivity can represent an emotional state similar to, or approximating, happiness. As another example, an intensity 2244 in a range of negative affectivity can represent an emotional state similar to, or approximating, depression/sadness, whereas intensity 2246 in a range of positive affectivity can represent an emotional state similar to, or approximating, relaxation. As shown, intensities 2240 and 2242 are greater than that of intensities 2244 and 2246. Emotive formulation module 2223 is configured to transmit this information as affective state data 230 describing a predicted emotion of a user. An example of affective state manager 2220 is described as a affective state prediction unit of U.S. Provisional Patent Application No. 61/705,598 filed on Sep. 25, 2012, which is incorporated by reference herein for all purposes.

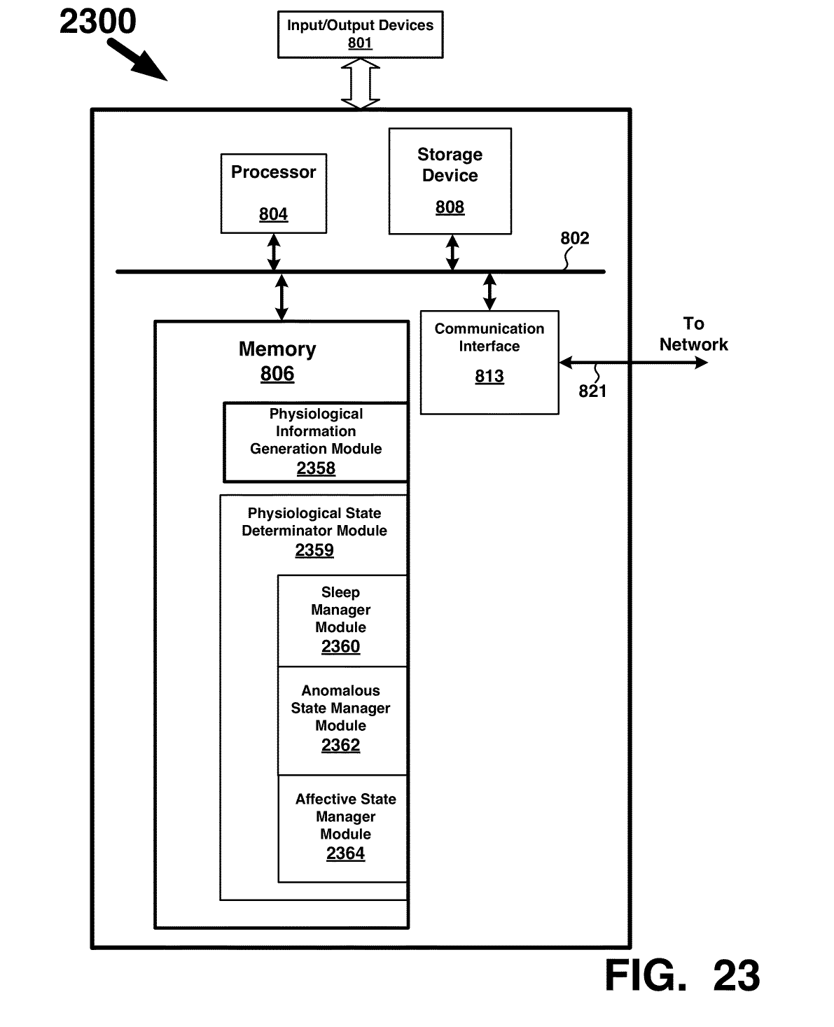

FIG. 23 illustrates an exemplary computing platform disposed in a wearable device in accordance with various embodiments. In some examples, computing platform 2300 may be used to implement computer programs, applications, methods, processes, algorithms, or other software to perform the above-described techniques, and can include similar structures and/or functions as set forth in FIG. 8. But in the example shown, system memory 806 can include various modules that include executable instructions to implement functionalities described herein. In the example shown, system memory 806 includes a physiological information generator 2358 configured to determine physiological information relating to a user that is wearing a wearable device, and a physiological state determinator 2359. Physiological state determinator 2359 can include a sleep manager module 2360, anomalous state manager module 2362, and an affective state manager module 2364, any of which can be configured to provide one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

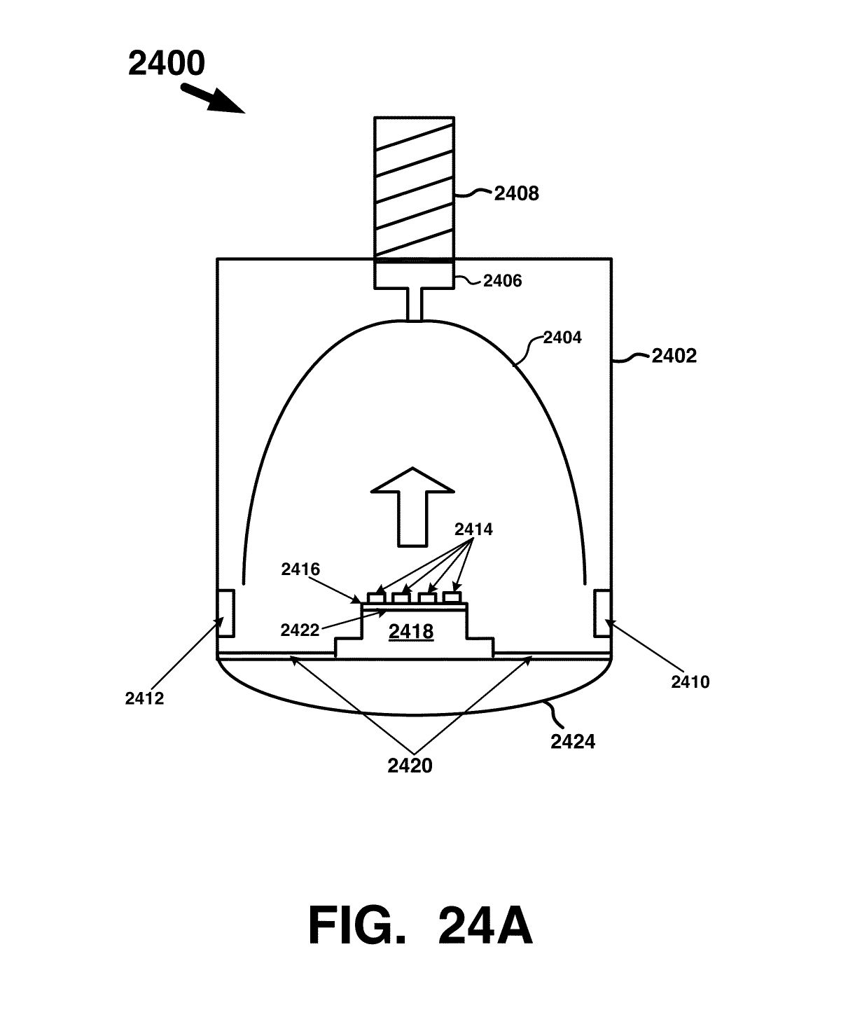

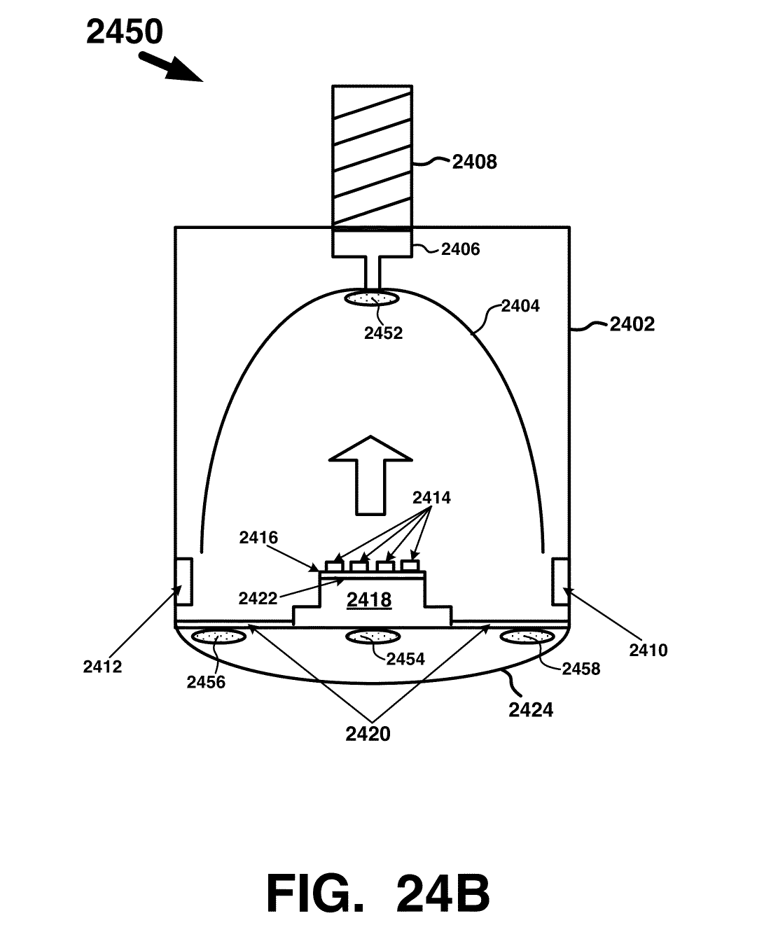

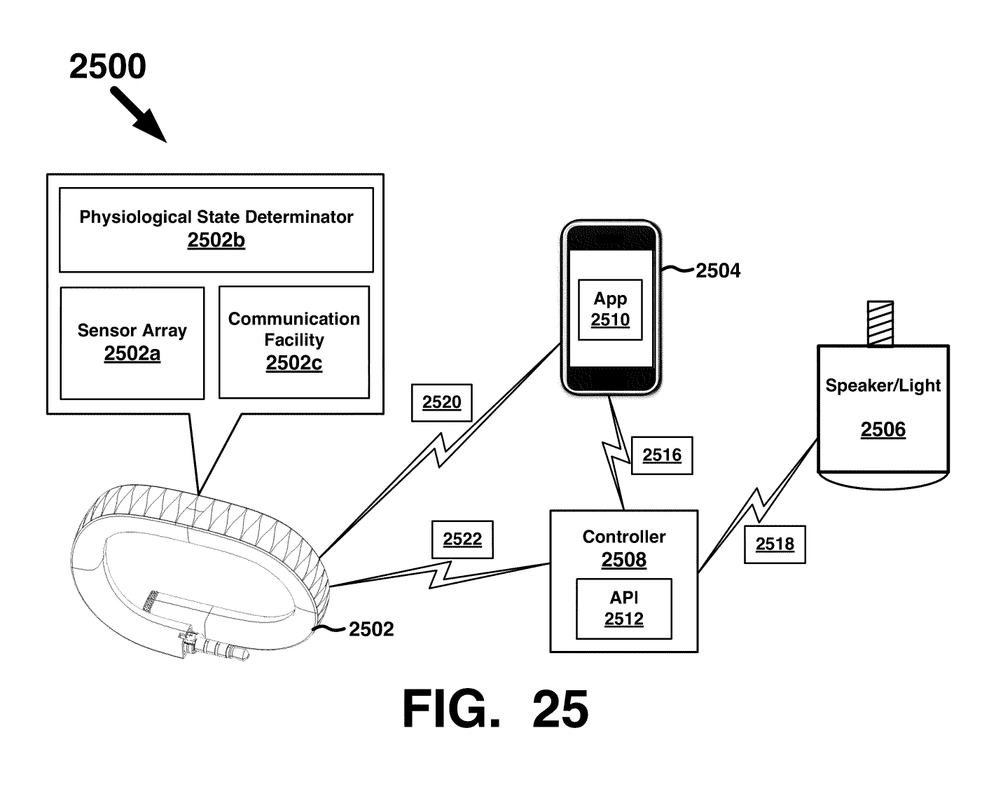

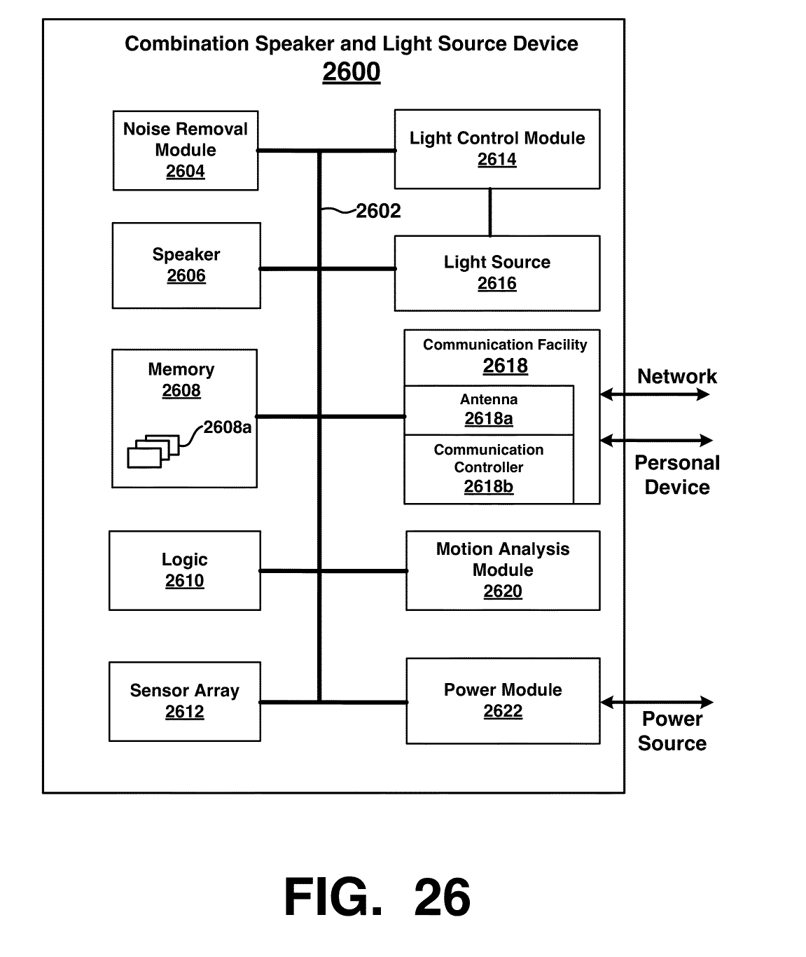

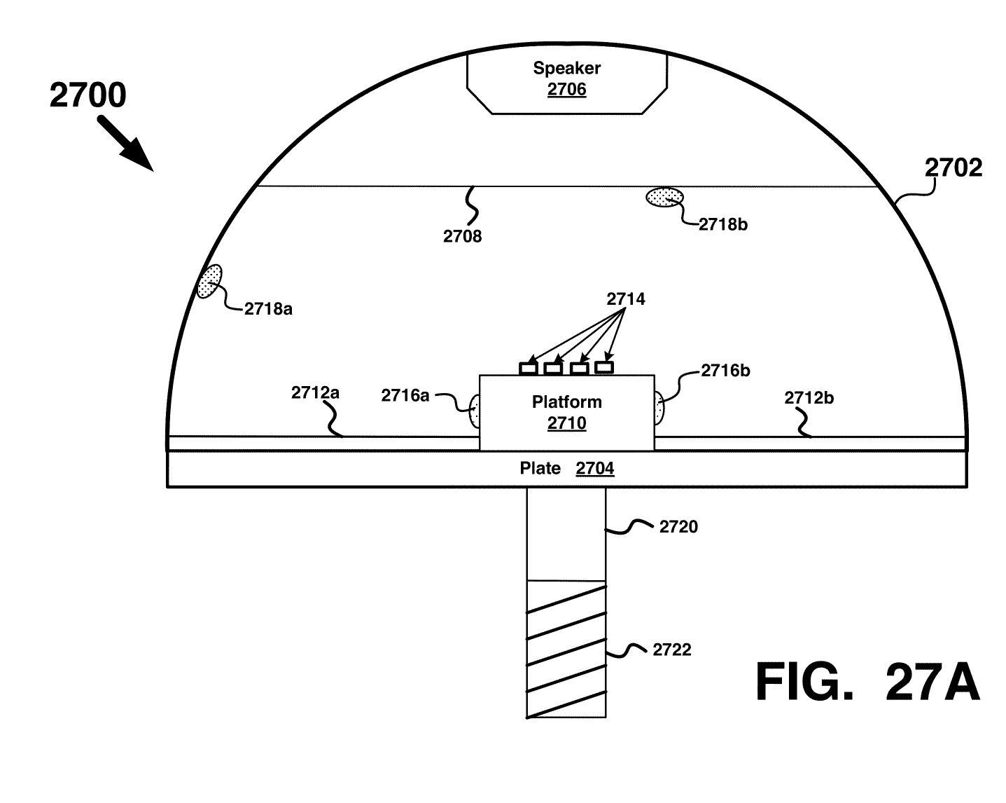

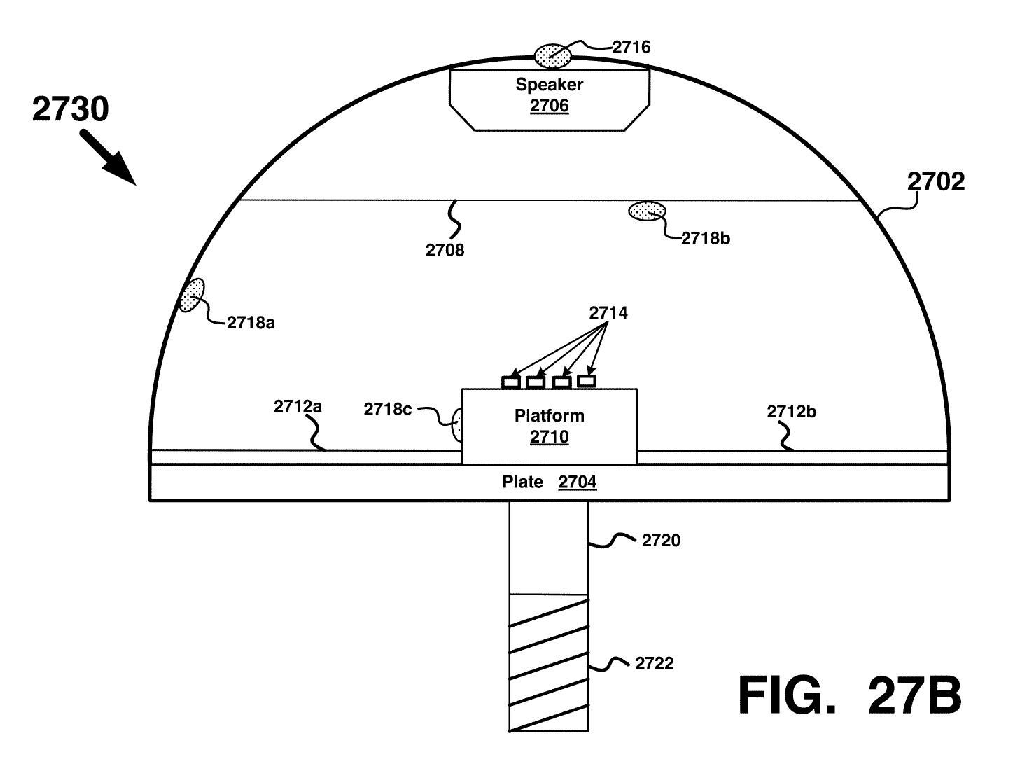

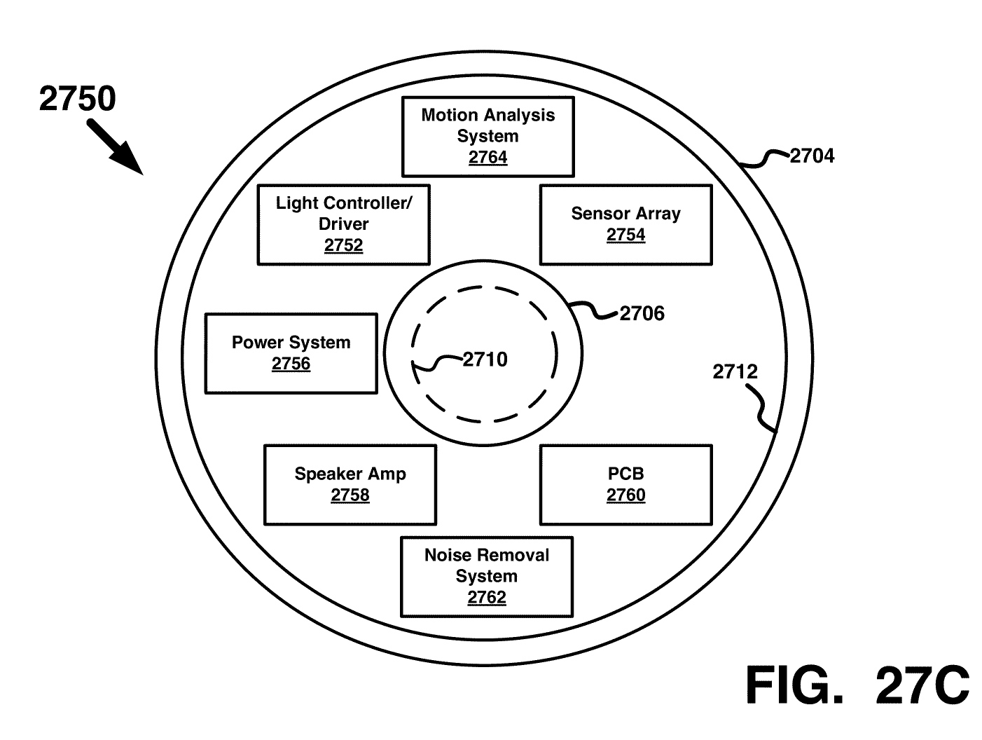

FIGS. 24A-24B illustrate exemplary combination speaker and light source devices powered using a light socket. Here, device 2400 includes housing 2402, parabolic reflector 2404, positioning mechanism 2406, light socket connector 2408, passive radiators 2410-2412, light source 2414, circuit board (PCB) 2416, speaker 2418, frontplate 2420, backplate 2422 and optical diffuser 2424. In some examples, device 2400 may be implemented as a combination speaker and light source (hereinafter “speaker-light device”), including a controllable light source (i.e., light source 2414) and a speaker system (i.e., speaker 2418). In some examples, light source 2414 may be configured to provide adjustable and controllable light, including an on or off state, varying colors, brightness, and irradiance patterns, without limitation. In some examples, light source 2414 may be controlled using a controller or control interface (not shown) in data communication with light source 2414 (i.e., using a communication facility implemented on PCB 2416) using a wired or wireless network (e.g., power line standards (e.g., G.hn, HomePlugAV, HomePlugAV2, IEEE1901, or the like), Ethernet, WiFi (e.g., 802.11a/b/g/n/ac, or the like), Bluetooth®, or the like). In some examples, light source 2414 may be implemented using one or more light emitting diodes (LEDs) coupled to PCB 2416. For example, light source 2414 may include different colored LEDs (e.g., red, green, blue, white, and the like), which may be used individually or in combination to produce a broad spectrum of colored light, as well as various hues. Each LED, or set of LEDs, may be controlled independently to generate various patterns. In other examples, light source 2414 may be implemented using a different type of light source (e.g., incandescent, light emitting electrochemical cells, halogen, compact fluorescent, or the like). In some examples, PCB 2416 may be bonded or otherwise mounted to backplate 2422, which may be coupled to a driver (not shown) for speaker 2418, to provide a heatsink for light source 2414. In some examples, PCB 2416 may provide a control signal to light source 2414, for example, to turn light source 2414 on and off, or control various characteristics associated with light source 2414 (e.g., amount, amplitude, brightness, color, quality, of light, or the like). In some examples, PCB 2416 may be configured to implement one or more control modules or systems (e.g., motion analysis module 2620 and noise removal module 2604 in FIG. 26, motion analysis system 2764 and noise removal system 2762 in FIG. 27C, motion analysis module 2810 and noise removal module 2812 in FIG. 4, and the like), as described herein, to generate a control signal configured to change a light characteristic associated with light output by light source 2414. In some examples, light source 2414 may direct light towards parabolic reflector 2404, as shown. In some examples, parabolic reflector 2404 may be configured to direct light from light source 2414 towards a front of housing 2402 (i.e., towards frontplate 2420 and optical diffuser 2424), which may be transparent. In some examples, parabolic reflector 2404 may be movable (e.g., turned, rotated, shifted, repositioned, or the like) using positioning mechanism 2406, either manually or electronically, for example, using a remote control in data communication with circuitry implemented in positioning mechanism 2406. For example, parabolic reflector 2404 may be moved to change an output light irradiation pattern. In some examples, parabolic reflector 2404 may be acoustically transparent such that additional volume within housing 2402 (i.e., around and outside of parabolic reflector 2404) may be available for acoustic use with a passive radiation system (e.g., including passive radiators 2410-2412, and the like).

In some examples, light socket connector 2408 may be configured to be coupled with a light socket (e.g., standard Edison screw base, as shown, bayonet mount, bi-post, bi-pin, or the like) for powering (i.e., electrically) device 2400. In some examples, light socket connector 2408 may be coupled to housing 2402 on a side opposite to optical diffuser 2424 and/or speaker 2418. In some examples, housing 2402 may be configured to house one or more of parabolic reflector 2404, positioning mechanism 2406, passive radiators 2410-2412, light source 2414, PCB 2416, speaker 2418 and frontplate 2420. Electronics (not shown) configured to support control, audio playback, light output, and other aspects of device 2400, may be mounted anywhere inside or outside of housing 2402, for example on a plate (e.g., plate 2704 in FIGS. 27A-27C). In some examples, light socket connector 2408 may be configured to receive power from a standard light bulb or power connector socket (e.g., E26 or E27 screw style, T12 or GU4 pins style, or the like), using either or both AC and DC power. In some examples, device 2400 also may be implemented with an Ethernet connection.

In some examples, speaker 2418 may be suspended in the center of frontplate 2420, which may be sealed. In some examples, frontplate 2420 may be transparent and mounted or otherwise coupled with one or more passive radiators. In some examples, speaker 2418 may be configured to be controlled (e.g., to play audio, to tune volume, or the like) remotely using a controller (not shown) in data communication with speaker 2418 using a wired or wireless network. In some examples, housing 2402 may be acoustically sealed to provide a resonant cavity when combined with passive radiators 2410-2412 (or other passive radiators, for example, disposed on frontplate 2420 (not shown). In other examples, radiators 2410-2412 may be disposed on a different internal surface of housing 2402 than shown. The combination of an acoustically sealed housing 2402 with one or more passive radiators (e.g., passive radiators 2410-2412) improves low frequency audio signal reproduction, while optical diffuser 2424 may be acoustically transparent, thus sound from speaker 2418 may be projected out of a front end of housing 2402 through optical diffuser 2424. In some examples, optical diffuser 2424 may be configured to be waterproof (e.g., using a seal, chemical waterproofing material, and the like). In some examples, optical diffuser 2424 may be configured to spread light (i.e., reflected using parabolic reflector 2404) evenly as light exits housing 2402 through a transparent frontplate 2420. In some examples, optical diffuser 2424 may be configured to be acoustically transparent in a frequency selective manner (i.e., acoustically transparent, or designed to not impede sound waves, in certain selected frequencies), functioning as an additional acoustic chamber volume (i.e., forming an acoustic chamber volume with a front end of housing 2402, as defined by frontplate 2420, as part of a passive radiator system including housing 2402, radiators 2410-2412, and other components of device 2400). In other examples, the quantity, type, function, structure, and configuration of the elements shown may be varied and are not limited to the examples provided.

In FIG. 24B, speaker-light device 2450 also may include housing 2402, parabolic reflector 2404, positioning mechanism 2406, light socket connector 2408, passive radiators 2410-2412, light source 2414, circuit board (PCB) 2416, speaker 2418, frontplate 2420, backplate 2422 and optical diffuser 2424, as well as sensors 2452-2458. In some examples, sensor 2454 may comprise an optical or light sensor (e.g., infrared (IR), LED, luminosity, photoelectric, photodetector, photodiode, electro-optical, optical position sensor, fiber optic, and the like), and may be disposed, placed, coupled, or otherwise located, on a side of speaker 2418 or frontplate 2420 opposite to light source 2414, such that sensor 2454 is shielded from light from light source 2414 being dispersed by parabolic reflector 2404, and said light will not interfere with the ability of sensor 2454 to detect light from a source other than light source 2414. In some examples, sensors 2456-2458 may comprise one or more acoustic sensors (e.g., microphone, acoustic vibration sensor, skin-surface microphone, microelectromechanical systems (MEMS), and the like), and may be disposed, placed, coupled, or otherwise located, on a side of housing 2402 or frontplate 2420, away from a direction of audio output by speaker 2418 in order to minimize any interference by speaker 2418 with the ability of sensors 2456-2458 to detect ambient sounds, speech, or acoustic vibrations other than said audio output by speaker 2418. In some examples, one or more of sensors 2452-2458 may comprise other types of sensors (e.g., chemical (e.g., CO₂, O₂, CO, and the like), temperature, motion, and the like), as described herein. In other examples, the quantity, type, function, structure, and configuration of the elements shown may be varied and are not limited to the examples provided.

FIG. 25 illustrates an exemplary system for manipulating a combination speaker and light source according to a physiological state determined using sensor data. Here, system 2500 includes wearable device 2502, mobile device 2504, speaker-light 2506 and controller 2508. Like-numbered and named elements may describe the same or substantially similar elements as those shown in other descriptions. In some examples, wearable device 2502 may include sensor array 2502a, physiological state determinator 2502b and communication facility 2502c. As used herein, “facility” refers to any, some, or all of the features and structures that are used to implement a given set of functions. In some examples, communication facility 2502c may be configured to communicate (i.e., exchange data) with other devices (e.g., mobile device 2504, controller 2508, or the like), for example, using short-range communication protocols (e.g., Bluetooth®, ultra wideband, NFC, or the like) or longer-range communication protocols (e.g., satellite, mobile broadband, GPS, WiFi, and the like). In some examples, physiological state determinator 2502b may be configured to output data (i.e., state data, as described herein) associated with a physiological state (e.g., states of sleep, wakefulness, a normative physiological state, a deviation from a normative physiological state, an affective state, or the like), which physiological state determinator 2502b may be configured to generate using sensor data captured using sensor array 2502a, as described herein. For example, physiological state determinator 2502b may be configured to generate state data 2520-2522. In some examples, wearable device 2502 may be configured to communicate state data 2520 to mobile device 2504 using communication facility 2502c. In some examples, wearable device 2502 may be configured to communicate state data 2522 to controller 2508 using communication facility 2502c.

In some examples, mobile device 2504 may be configured to run application 2510, which may be configured to receive and process state data 2520 to generate data 2516. In some examples, data 2516 may include light data (i.e., light characteristic data, as described herein) associated with light patterns congruent with state data provided by wearable device 2502 (e.g., state data 2520 and the like). For example, where state data 2520 indicates a predetermined or designated wake up time, application 2510 may generate light data associated with a gradual brightening of a light source implemented in speaker-light 2506. In another example, where state data 2520 indicates a sleep or resting state, application 2510 may generate light data associated with a dimming of a light source implemented in speaker-light 2506. In still other examples, light data generated by application 2510 may be associated with a light pattern, a level of light, or the like, for example, depending on an activity (e.g., dancing, meditating, exercising, walking, sleeping, or the like) indicated by state data 2520. In some examples, data 2516 may include audio data (i.e., audio characteristic data, as described herein) associated with audio output congruent with state data provided by wearable device 2502 (e.g., state data 2520 and the like). For example, application 2510 may be configured to generate audio data associated with playing audio content (e.g., a playlist, an audio file including animal noises, an audio file including a voice recording, or the like) associated with an activity (e.g., dancing, meditating, exercising, walking, sleeping, or the like) using a speaker implemented in speaker-light 2506 when state data 2520 indicates said activity is beginning or ongoing. In another example, application 2510 may be configured to generate audio data associated with adjusting white noise or other ambient noise (e.g., to improve sleep quality, to ease a waking up process, to match a mood or activity, or the like) output by a speaker implemented in speaker-light 2506 when state data 2520 indicates an analogous physiological state. In other examples, application 2510 may be implemented directly in controller 2508, for example, using state data 2522, which may include the same or similar kinds of data associated with physiological states as described herein in relation to state data 2520. In some examples, controller 2508 may be configured to generate one or more control signals, for example, using API 2512, and to send said one or more control signals to speaker-light 2506 to adjust a light source and/or speaker. For example, the one or more control signals may be configured to cause a light source to dim or brighten. In another example, the one or more control signals may be configured to cause the light source to display a light pattern. In still another example, the one or more control signals may be configured to cause a speaker to play audio content. In yet another example, the one or more control signals may be configured to cause a speaker to play ambient noise. In other examples, the quantity, type, function, structure, and configuration of the elements shown may be varied and are not limited to the examples provided.

FIG. 26 illustrates an exemplary architecture for a combination speaker and light source device. Here, combination speaker and light source device (i.e., speaker-light device) 2600 includes bus 2602, noise removal module 2604, speaker 2606, memory 2608, logic 2610, sensor array 2612, light control module 2614, light source 2616, communication facility 2618, motion analysis module 2620, and power module 2622. Like-numbered and named elements may describe the same or substantially similar elements as those shown in other descriptions. In some examples, sensor array 2612 may include one or more of a motion sensor (e.g., accelerometer, gyroscopic sensors, optical motion sensors (e.g., laser or LED motion detectors, such as used in optical mice), magnet-based motion sensors (e.g., detecting magnetic fields, or changes thereof, to detect motion), electromagnetic-based sensors, MEMS, and the like), a chemical sensor (e.g., carbon dioxide (CO2), oxygen (O₂), carbon monoxide (CO), airborne chemical, toxin, and the like), a temperature sensor (e.g., thermometer, temperature gauge, IR thermometer, resistance thermometer, heat flux sensor, and the like), humidity sensor, passive IR sensor, ultrasonic sensor, proximity sensor, pressure sensor, light sensors and acoustic sensors, as described herein, and the like. In some examples, noise removal module 2604 may be configured to remove audio output from speaker 2606 from sounds (i.e., acoustics) being captured using an acoustic sensor in sensor array 2612. For example, noise removal module 2604 may be configured to subtract the output from speaker 2606 from the acoustic input to sensor array 2612 to determine ambient sound in a room or other environment surrounding speaker-light device 2600. In other examples, noise removal module 2604 may be configured to remove a different set of known acoustic noise (e.g., permanent ambient noise, frequency-selected noise, ambient noise to isolate speech or a speech command, and the like). In some examples, motion analysis module 2620 may be configured to generate movement data using sensor data captured by sensor array 2612, the movement data indicating an identity (i.e., by a motion signature or motion fingerprint) of, or activity or gesture (e.g., fingerpoint, arm wave, hand wave, thumbs up, and the like) being performed by, a person in a room or other environment surrounding speaker-light device 2600. Techniques associated with determining an activity using sensor data are described in co-pending U.S. patent application Ser. No. 13/433,204 (Attorney Docket No. ALI-013CIP1), filed Mar. 28, 2012, and techniques associated with determining, and identifying a person with, a motion fingerprint or signature are described in co-pending U.S. patent application Ser. No. 13/181,498 (Attorney Docket No. ALI-018), filed Jul. 12, 2011, all of which are incorporated by reference herein in their entirety for all purposes. In some examples, motion analysis module 2620 also may be configured to determine a level, amount, or type of motion in a room or environment, and cross-reference such information with data generated by communication facility 2618 indicating a number of personal devices, and thus a number of people, in said room or environment, to determine a nature of a setting (e.g., social, private, a single person using a single media device, two or more people using separate media devices, a single person using multiple media devices, a set of people using a single media device, a single person resting or sleeping, an adult and a baby resting or sleeping, and the like). In some examples, said activity or gesture may cause speaker-light device 2600, for example, based on profile data 2608a stored in memory 2608, to change or modify a light characteristic (e.g., color, brightness, hue, pattern, amplitude, frequency, and the like) associated with light output by light source 2616 and/or an audio characteristic (e.g., volume, perceived loudness, amplitude, sound pressure, noise reduction, frequency selection, normalization, and the like) associated with audio output by speaker 2606. In some examples, light characteristics may be modified using light control module 2614, which may include a light controller and a driver. In other examples, profile data 2608a may associate a light characteristic with an audio characteristic, thus causing light control module 2614 to direct a control signal (i.e., light control signal) to light source 2616 to modify a light characteristic associated with light being output by light source 2616 in response to audio being output by speaker 2606, thus correlating a light output with an audio output (e.g., flashing lights or laser light patterns being output in coordination with loud, techno, or other fast tempo, music with hard beats; dim, warm, steady light being output in coordination with slow, soft, instrumental music; and the like). In some examples, speaker 2606 may be implemented as a speaker system, including one or more of a woofer, a tweeter, other drivers, a passive or hybrid radiation system, reflex port, and the like. In other examples, the quantity, type, function, structure, and configuration of the elements shown may be varied and are not limited to the examples provided.

In some examples, profile data 2608a may comprise activity-related profiles indicating optimal lighting and acoustic output (i.e., light and audio characteristics) for an activity (e.g., warm, yellow light and/or soft background music for an evening social setting; low, yellow light and/or white noise for resting or sleeping; bright, blue-white light with no music or sounds for working or studying during the day). In some examples, profile data 2608a also may comprise identity-related profiles for one or more users, the identity-related profiles including preference data indicating a user's preferences for light characteristics and audio characteristics in a room or other environment surrounding speaker-light device 2600. Such preference data may be uploaded or saved to speaker-light device 2600, for example, from a personal device (e.g., wearable device, mobile device, portable device, or other device attributable to a user or owner) using communication facility 2618, or it may be learned by speaker-light device 2600 over a period of time through manual manipulation by a user identified using motion analysis module 2620 (e.g., gesture command, motion fingerprint, or the like), communication facility 2618 (i.e., identity data received from a personal device), or the like. In other examples, profile data 2608a may include data correlating light and audio characteristics with other types of sensor data and derived data (e.g., a visual or audio alarm for toxic chemical levels or smoke, light and audio characteristics associated with one or more hand gestures or speech commands, and the like). In some examples, a personal device may be configured to implement an application configured to provide an interface for inputting, uploading, or otherwise indicating, a user's or owner's lighting and audio preferences.

In some examples, communication facility 2618 may include antenna 2618a and communication controller 2618b, and may be implemented as an intelligent communication facility, techniques associated with which are described in co-pending U.S. patent application Ser. No. 13/831,698 (Attorney Docket No. ALI-191CIP1), filed Mar. 15, 2013, which is incorporated by reference herein in its entirety for all purposes. As used herein, “facility” refers to any, some, or all of the features and structures that are used to implement a given set of functions. In some examples, communication controller 2618b may include one or both of a short-range communication controller (e.g., Bluetooth, NFC, ultra wideband, and the like) and longer-range communication controller (e.g., satellite, mobile broadband, GPS, WiFi, and the like). In some examples, communication facility 2618 may be configured to ping, or otherwise send a message or query to, a network or personal device detected using antenna 2618a, for example, to obtain preference data or other data associated with a light characteristic or audio characteristic, as described herein. In some examples, antenna 2618a may be implemented as a receiver, transmitter, or transceiver, configured to detect and generate radio waves, for example, to and from electrical signals. In some examples, antenna 2618a may be configured to detect radio signals across a broad spectrum, including licensed and unlicensed bands. In some examples, communication facility may include other integrated circuitry (not shown) for enabling advanced communication capabilities (e.g., Bluetooth® Low Energy system on chip (SoC), and the like).

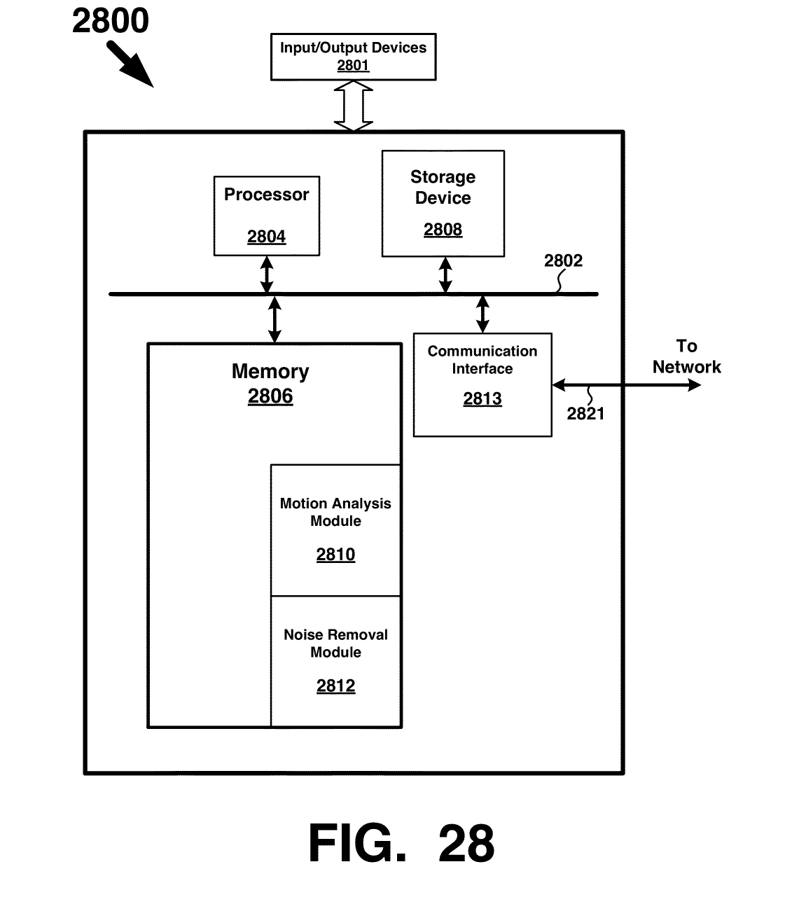

In some examples, logic 2610 may be implemented as firmware or application software that is installed in a memory (e.g., memory 2608, memory 2806 in FIG. 28, or the like) and executed by a processor (e.g., processor 2804 in FIG. 28). Included in logic 2610 may be program instructions or code (e.g., source, object, binary executables, or others) that, when initiated, called, or instantiated, perform various functions. In some examples, logic 2610 may provide control functions and signals to other components of speaker-light device 2600, including to speaker 2606, light control module 2614, communication facility 2618, sensor array 2612, or other components. In some examples, one or more of the components of speaker-light device 2600, as described herein, may be connected and implemented using a PCB (e.g, PCB 2416 from FIGS. 24A-24B, as described herein). In some examples, power module 2622 may include a power converter, a transformer, and other electrical components for supplying power to other elements of speaker-light device 2600. In some examples, power module 2622 may be coupled to a light socket connector (e.g., light socket connector 2408 in FIGS. 24A-24B, light socket connector 2722 in FIGS. 27A-27B, and the like) to retrieve electrical power from a power source. In other examples, the quantity, type, function, structure, and configuration of the elements shown may be varied and are not limited to the examples provided.

FIGS. 27A to 27B illustrate side-views of exemplary combination speaker and light source devices. Here, speaker-light device 2700 includes enclosure 2702 and plate 2704 forming a housing, speaker 2706, speaker enclosure 2708, platform 2710, light source 2714, electronics 2712a-2712b, light sensors 2716a-2716b, acoustic sensors 2718a-2718b, extension structure 2720 and light socket connector 2722. Like-numbered and named elements may describe the same or substantially similar elements as those shown in other descriptions. In some examples, platform 2710 may be configured to couple light source 2714 to plate 2704. In other examples, platform 2710 may be configured to couple light source 2714 to a different part of a housing (i.e., enclosure 2702). In some examples, platform 2710 may comprise a terminal configured to receive, or be coupled to, light source 2714, and to provide control signals to light source 2714 (i.e., light source 2714 may be plugged into said terminal). In some examples, a terminal also may be coupled to a light controller (e.g., light control module 2614 in FIG. 26, light controller/driver 2752 in FIG. 27C, or the like), the terminal configured to receive a control signal (i.e., a light control signal) configured to modify a light characteristic. In some examples, speaker enclosure 2708 may be disposed or located between speaker 2706 and light source 2714. In some examples, speaker enclosure 2708 may be formed using a clear material allowing light from light source 2714 to pass through. In some examples, speaker enclosure 2708 may be formed using an acoustically opaque material such that audio output from speaker 2706 does not travel through speaker enclosure 2708, thus shielding acoustic sensors 2718a-2718b from said audio output. In other examples, speaker enclosure 2708 may be formed using an acoustically transparent material, and the acoustics captured by acoustic sensors 2718a-2718b may be later processed by a noise removal system (e.g., noise removal module 2604 in FIG. 26, noise removal system 2762 in FIG. 27C, or the like), as described herein, to remove or subtract audio output from speaker 2706 to derive data attributable to ambient sounds not created by speaker-light device 2700. In still other examples, acoustic sensors 2718a-2718b may be configured to face away from speaker 2706, for example at an angle, in order to minimize the amount of audio output from speaker 2706 being captured by acoustic sensors 2718a-2718b. In some examples, light sensors 2716a-2716b may be located on platform 2710 underneath, or otherwise facing away from, light source 2714, to minimize the amount of light from light source 2714 being captured by light sensors 2716a-2716b. In other examples, light sensors and acoustic sensors may be implemented in speaker-light device 2700 differently, such as shown in FIG. 27B, and described below.

In some examples, enclosure 2702 may be hemispherical or substantially hemispherical in shape. In some examples, enclosure 2702 may be partially opaque, thus allowing light from light source 2714 to be directed out of enclosure 2702 through a portion that is not opaque (e.g., translucent or transparent). In other examples, enclosure 2702 may be partially or wholly translucent and/or transparent.

In some examples, platform 2710 and electronic components 2712a-2712b may be coupled to plate 2704. In some examples, platform 2710 also may be coupled to light source 2714, and may include a heatsink for light source 2714. In some examples, extension structure 320 may be included to couple plate 2704 to light socket connector 2722, where speaker-light device 2700 is configured to be plugged, inserted, or otherwise coupled to a recessed light or power connector socket. In some examples, electronics 2712a-2712b may include a motion analysis system, a power system, a speaker amplifier, a noise removal system, a PCB, and the like, as described herein in FIG. 27C.

In some examples, one or more passive radiators (not shown) may be implemented within enclosure 2702, either within an acoustically opaque speaker enclosure 2708 or to both sides of an acoustically transparent speaker enclosure 2708, to form a passive radiation system for speaker 2706. In other examples, the quantity, type, function, structure, and configuration of the elements shown may be varied and are not limited to the examples provided.

FIG. 27B illustrates a side-view of another exemplary speaker-light device. Here, speaker-light device 2730 includes light sensor 2716 and acoustic sensors 2718a-2718c, among other components described above. Like-numbered and named elements may describe the same or substantially similar elements as those shown in other descriptions. In some examples, light sensors 2716a-2716b (e.g., infrared, LED, or the like, as described herein) may be disposed or located on a side of speaker 2706 facing away from light source 2714, speaker 2706 thus shielding light sensor 2716 from detecting light output from light source 2714. In other examples, the quantity, type, function, structure, and configuration of the elements shown may be varied and are not limited to the examples provided.

FIG. 27C illustrates a top-view of an exemplary combination speaker and light source device. Here, speaker-light device 2750 includes housing 2704, speaker 2706, platform 2710 being hidden by speaker 2706, and electronics 2712, including light controller/driver 2752, sensor array 2754, power system 2756, speaker amplifier 2758, PCB 2760, noise removal system 2762, and motion analysis system 2764. Like-numbered and named elements may describe the same or substantially similar elements as those shown in other descriptions. In some examples, housing 2704 may include a hemispherical enclosure coupled to a plate as described herein. In other examples, housing 2704 may be formed in a different shape than shown and described herein (e.g., cube, rectangular box, pill-shape, ovoid, bulb-shaped, and the like). In some examples, light controller/driver 2752 may be configured to provide control signals to a light source (e.g., light source 2414 in FIGS. 24A-24B, light source 2616 in FIG. 26, light source 2714 in FIGS. 27A-27B, and the like) to modify a characteristic of light being output (e.g., dim, brighten, change color, change hue, turn on, turn off, start/stop or change a light pattern, and the like). In some examples, power system 2756 may include circuitry configured to operate a power module (e.g., power module 2622 in FIG. 26, and the like) for accessing power from a power source, for example, using a light connector socket, as described herein. In some examples, sensor array 2754 may include various sensors, as described herein, and may be configured to provide sensor data to motion analysis system 2764 and noise removal system 2762 for further processing, as described herein. In some examples, motion analysis system may include circuitry configured to operate a motion analysis module (e.g., motion analysis module 2620 in FIG. 26, or the like), as described herein. In some examples, noise removal system 2762 may include circuitry configured to operate a noise removal module (e.g., noise removal module 2604 in FIG. 26, or the like), as described herein. In other examples, the quantity, type, function, structure, and configuration of the elements shown may be varied and are not limited to the examples provided.

FIG. 28 illustrates an exemplary computing platform disposed in or associated with a combination speaker and light source device. Like-numbered and named elements may describe the same or substantially similar elements as those shown in other descriptions. In some examples, computing platform 2800 may be used to implement computer programs, applications, methods, processes, algorithms, or other software to perform the above-described techniques, and can include similar structures and/or functions as set forth in FIGS. 8 and 23. In the example shown, system memory 2806 can include various modules that include executable instructions to implement functionalities described herein. In the example shown, system memory 2806 includes a motion analysis module 2810 configured to analyze sensor data and generate movement data associated with detected movement, as described herein. Also shown is noise removal module 2812 configured to remove or subtract a known acoustic signal from acoustic sensor data captured by an acoustic sensor, as described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the structures and techniques described herein can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, speaker-light devices 2400, 2450, 2600, 2700, and 2750, including one or more components, can be implemented in one or more computing devices that include one or more circuits. Thus, at least one of the elements in FIGS. 24-27C can represent one or more components of hardware. Or, at least one of the elements can represent a portion of logic including a portion of circuit configured to provide constituent structures and/or functionalities. In other examples, the quantity, type, function, structure, and configuration of the elements shown may be varied and are not limited to the examples provided.

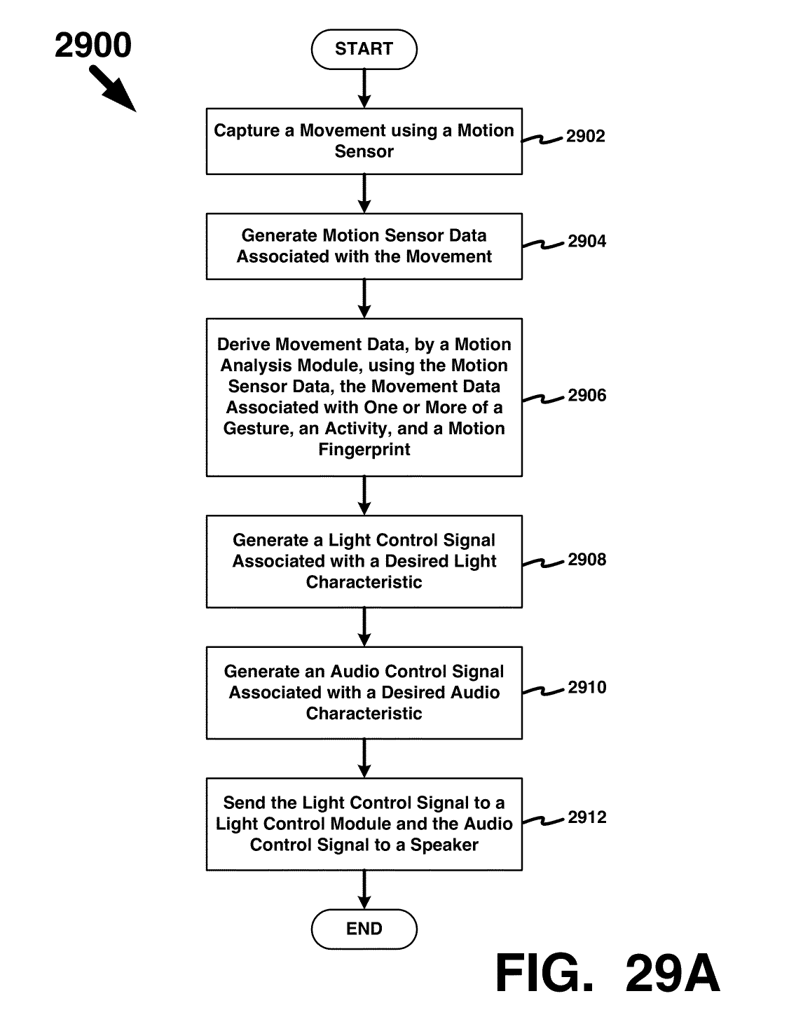

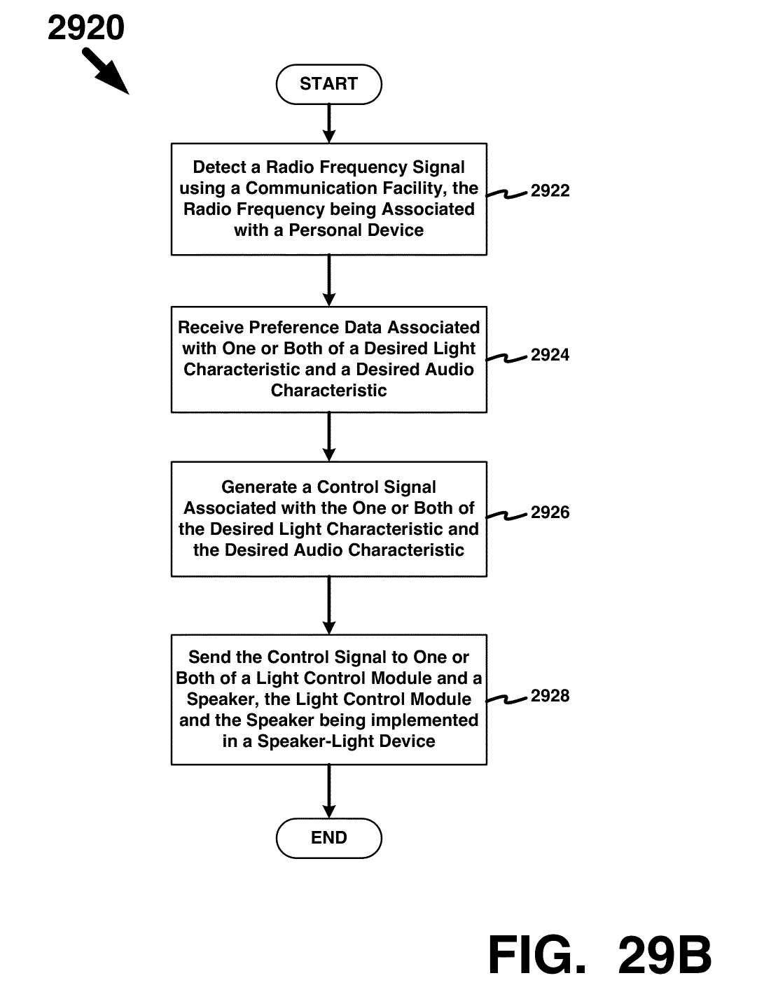

FIGS. 29A-29B illustrate exemplary flows for a combination speaker and light source device. Here, process 2900 begins with capturing a movement using a motion sensor (2902), for example, implemented in a speaker-light device, as described herein. In some examples, said movement may include an activity, a gesture (i.e., hand or arm gesture), or motion fingerprint (e.g., gait, arm swing, or the like). Said motion sensor may generate motion sensor data associated with the movement in response to said captured movement (2904). Then movement data may be derived by a motion analysis module using the motion sensor data, the movement data associated with one or more of a gesture, an activity, and a motion fingerprint (2906). In some examples, a motion analysis module may be implemented in said speaker-light device. In some examples, such movement data may be cross-referenced or correlated with preference data gathered from a personal device, for example, using process 2920 in FIG. 29B, as described herein, to determine one or more desired light characteristics and/or audio characteristics. In some examples, a motion analysis module may be configured to determine a desired light characteristic and/or audio characteristic. In some examples, a motion analysis module may be configured to perform the cross-reference of movement data with preference data. In other examples, a motion analysis module may provide movement data, or desired light and/or audio characteristic data associated with movement data, to another module to perform the cross-reference of movement data with preference data to determine or modify desired light and/or audio characteristic data. Once desired light characteristic data is determined (and in some cases, confirmed or modified according to preference data), a light control signal associated with said desired light characteristic may be generated (2908), the light control signal configured to modify a light output (e.g., brightness, color, hue, pattern, amplitude, frequency, on, off, or the like) by a light source, as described herein. In other examples, a determination may be made to keep light characteristics as they are (i.e., current light characteristics match determined desired light characteristics). Once desired audio characteristic data is determined (and in some cases, confirmed or modified according to preference data), an audio control signal associated with said desired audio characteristic may be generated (2910), the audio control signal configured to modify an audio output (e.g., volume, perceived loudness, amplitude, sound pressure, noise reduction, frequency selection, normalization, and the like) by a speaker, as described herein. The light control signal may be sent to a light control module, and the audio control signal sent to a speaker (2912), the light control module and the speaker being implemented in a speaker-light device. In other examples, the above-described process may be varied in steps, order, function, processes, or other aspects, and is not limited to those shown and described.

In FIG. 29B, process 2920 begins with detecting a radio frequency signal using a communication facility (i.e., implemented in a speaker-light device, as described herein), the radio frequency being associated with a personal device (2922). In some examples, a strength of a radio frequency signal may be used to determine a proximity of a personal device (i.e., wearable device, portable device, mobile device, or other device attributable to a user/owner). In some examples, a speaker-light device may be configured to ping, or otherwise send a query to, a personal device to obtain identity (i.e., identifying) data associated with a user or owner of said personal device, and said identity data may be associated with a profile stored in a memory implemented in a speaker-light device, as described herein. In some examples, a speaker-light device also may receive preference data associated with one or both of a desired light characteristic and a desired audio characteristic (2924). A control signal associated with the one or both of the desired light characteristic and the desired audio characteristic may be generated (2926), the control signal configured to modify a light output and/or audio output, as described herein. Once generated, the control signal may be sent to a light control module and/or a speaker, for example, being implemented in a speaker-light device (2928). In other examples, the above-described process may be varied in steps, order, function, processes, or other aspects, and is not limited to those shown and described.

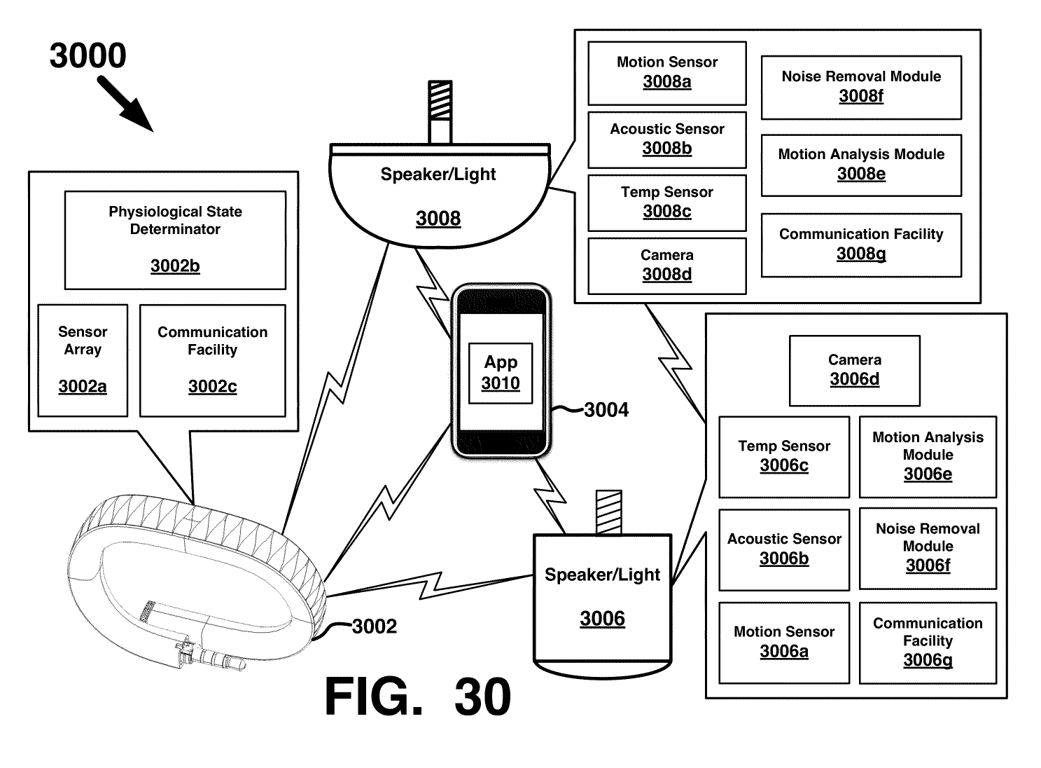

FIG. 30 illustrates an exemplary system for controlling a combination speaker and light source device according to a physiological state. Here, system 3000 includes wearable device 3002, mobile device 3004 and speaker-light devices 3006 and 3008. Like-numbered and named elements may describe the same or substantially similar elements as those shown in other descriptions. In some examples, wearable device 3002 may include sensor array 3002a comprised of one or more sensors, physiological state determinator 3002b configured to generate state data, and communication facility 3002c, as described herein. In some examples, mobile device 3010 may be configured to run application 3010 configured to generate, and send to speaker-light devices 3006 and 3008, a control signal (e.g., light control signal, audio control signal, and the like) configured to modify a light characteristic and/or audio characteristic. In some examples, various sensors (e.g., motion sensors 3006a and 3008a, acoustic sensors 3006b and 3008b, temperature sensors 3006c and 3008c, camera sensors 3006d and 3008d, and the like) implemented in speaker-light devices 3006 and 3008 also may provide raw sensor data to wearable device 3002 to inform physiological state determinator 3002b or to mobile device 3004 to inform application 3010, such raw sensor data being used to generate state data and control signal data, as described herein. In some examples, speaker-light devices 3006 and 3008 may send raw sensor data to wearable device 3002 or mobile device 3004 using communication facility 3006g and 3008g, respectively. In some examples, speaker-light devices 3006 and 3008 also may be configured to derive movement data, other motion-related data, or identity data, using motion analysis module 3006e and 3008e, and to provide movement data (i.e., using communication facilities 3006g and 3008g) to mobile device 3004 and/or wearable device 3002.

In some examples, speaker-light devices 3006 and 3008 also may be configured to derive acoustic or audio data using noise removal modules 3006f and 3008f, respectively. For example, noise removal module 3006f may derive audio data comprising ambient acoustic sound by subtracting or removing audio output (i.e., “noise”) from a speaker implemented by speaker-light 3006 from the total acoustic input captured by acoustic sensor 3006b. As used herein, “noise” refers to any sound or acoustic energy not desired to be included in audio data being derived for a purpose, which may include ambient noise in some examples, speaker output in other examples, and the like. In other examples, noise removal modules 3006f and 3008f may be configured to derive audio data comprising speech or a speech command by removing ambient acoustic sound and audio output from a speaker. In some examples, motion analysis modules 3006e and 3008e also may receive sensor data from acoustic sensors 3006b and 3008b, respectively, temperature data from temperature sensors 3006c and 3008c, respectively, image/video data from cameras 3006d and 3008d, respectively, and/or derived audio data from noise removal modules 3006f and 3008f, respectively. In some examples, motion analysis modules 3006e and 3008e also may cross-reference said sensor data with profiles (e.g., activity or preference profiles, or the like) stored in a memory (e.g., memory 2608 in FIG. 26, including profiles 2608a, and the like) to determine a desired light characteristic and/or a desired audio characteristic, and to generate one or more control signals associated with said desired light and/or audio characteristics. In other examples, speaker-light devices 3006 and 3008 may receive a control signals associated with a desired light and/or audio characteristic from wearable device 3002 and/or mobile device 3004 (i.e., as determined by application 3010).

In some examples, speaker-light devices 3006 and 3008 also may include a speaker and a light source (e.g., speaker 2606 and light source 2616 in FIG. 26, speaker 2418 and light source 2414 in FIGS. 24A-24B, and the like), along with other electrical components (e.g., light control module 2614 in FIG. 26, electronics 2712a-2712b in FIGS. 27A-27B, PCB 2760, light controller/driver 2752 and speaker amplifier 2758 in FIG. 27C, and the like) for controlling a speaker and a light source, as described herein. In some examples, speaker-light devices 3006 and 3008 may generate a light control signal for modifying a light characteristic, and an audio control signal for modifying an audio characteristic. In other examples, speaker-light devices 3006 and 3008 may receive one or more control signals from one or both of wearable device 3002 and mobile device 3004 for modifying a light characteristic and/or audio characteristic. In still other examples, the quantity, type, function, structure, and configuration of the elements shown may be varied and are not limited to the examples provided.

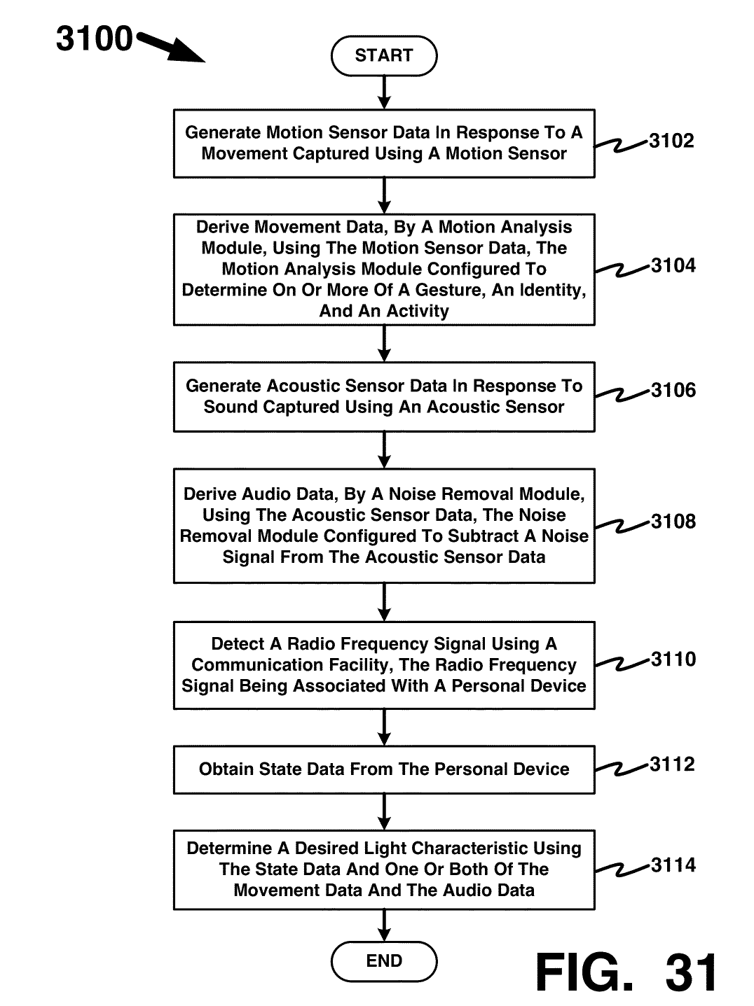

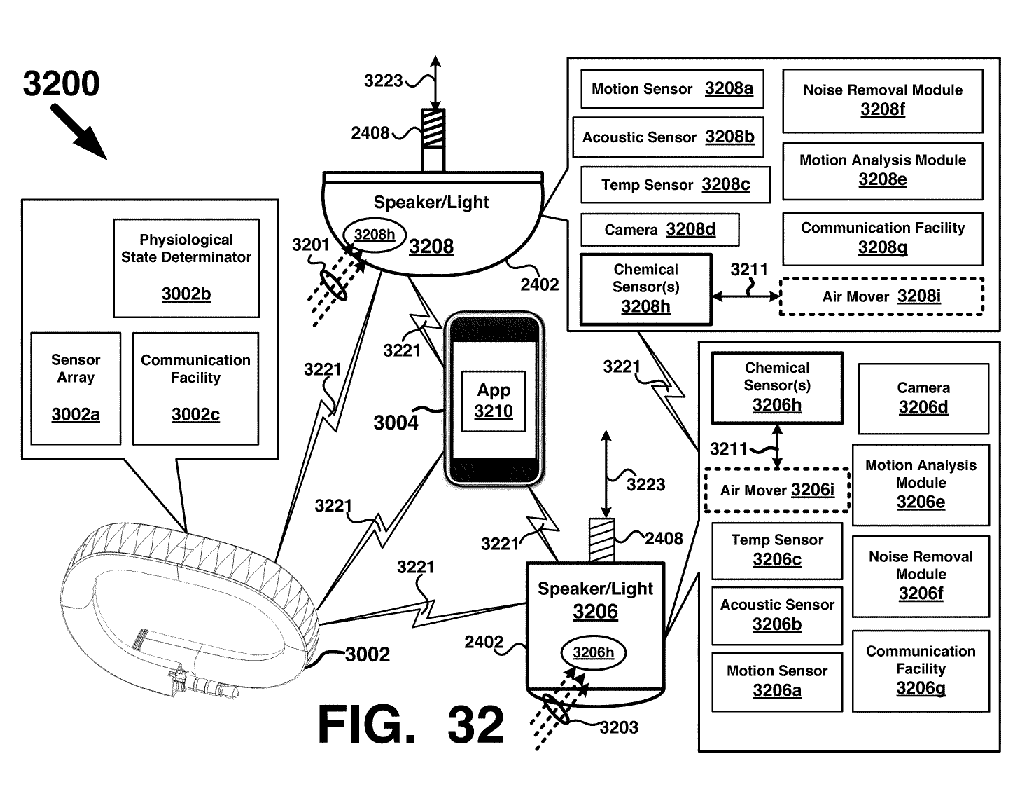

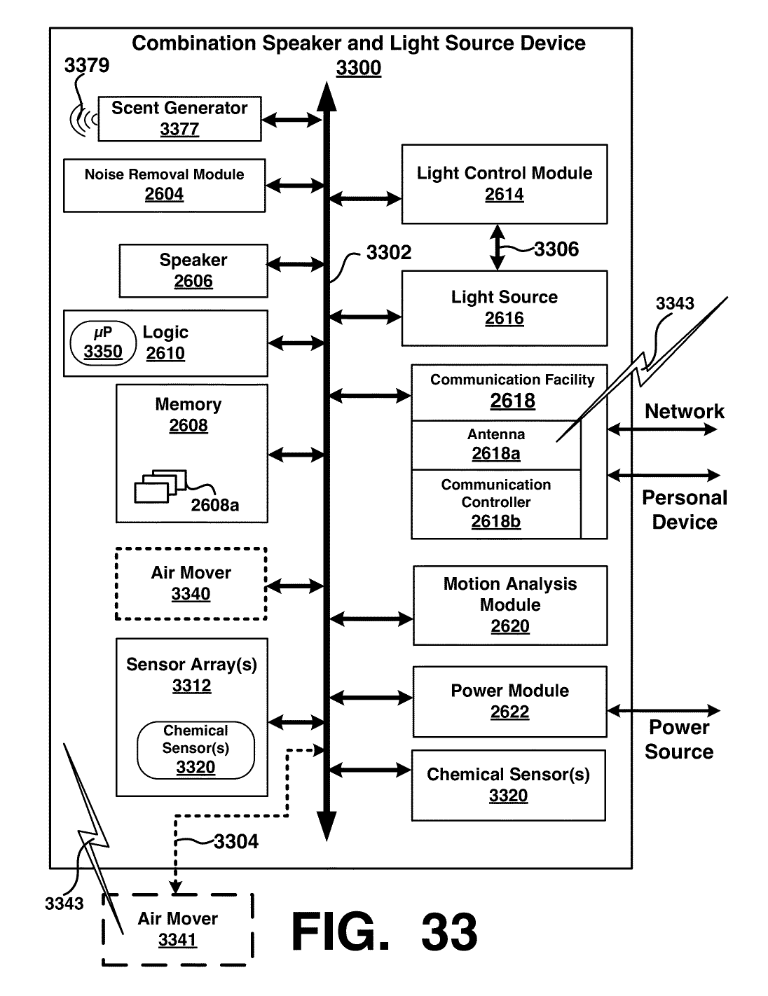

FIG. 31 illustrates an exemplary flow for controlling a combination speaker and light source device according to a physiological state. Here, process 3100 begins with generating motion sensor data in response to a movement captured using a motion sensor (3102). Using the motion sensor data, movement data may be derived by a motion analysis module configured to determine on or more of a gesture, an identity, and an activity (3104), as described herein. In some examples, acoustic sensor data may be generated in response to sound captured using an acoustic sensor (3106). Using the acoustic sensor data, audio data may be derived by a noise removal module configured to subtract a noise signal from the acoustic sensor data (3108), as described herein. In some examples, a radio frequency signal also may be detected using a communication facility, the radio frequency signal being associated with a personal device (3110). State data then may be obtained from the personal device (3112), and a desired light characteristic determined using the state data and one or both of the movement data and the audio data (3114). In some examples, a light control signal may be generated, the light control signal associated with the desired light characteristic, the light control signal configured to modify a light output by a light source, including a light color, hue, pattern, or the like. In some examples, a desired audio characteristic also may be determined using the state data and one or both of the movement data and the audio data. In some examples, an audio control signal also may be generated, the audio control signal configured to modify an audio output by a speaker. In some examples, one or more of the movement data, the audio data, and the state data may be sent to another device, such as a mobile device, as described herein, the mobile device configured to generate a light control signal and/or audio control signal. In other examples, the above-described process may be varied in steps, order, function, processes, or other aspects, and is not limited to those shown and described.

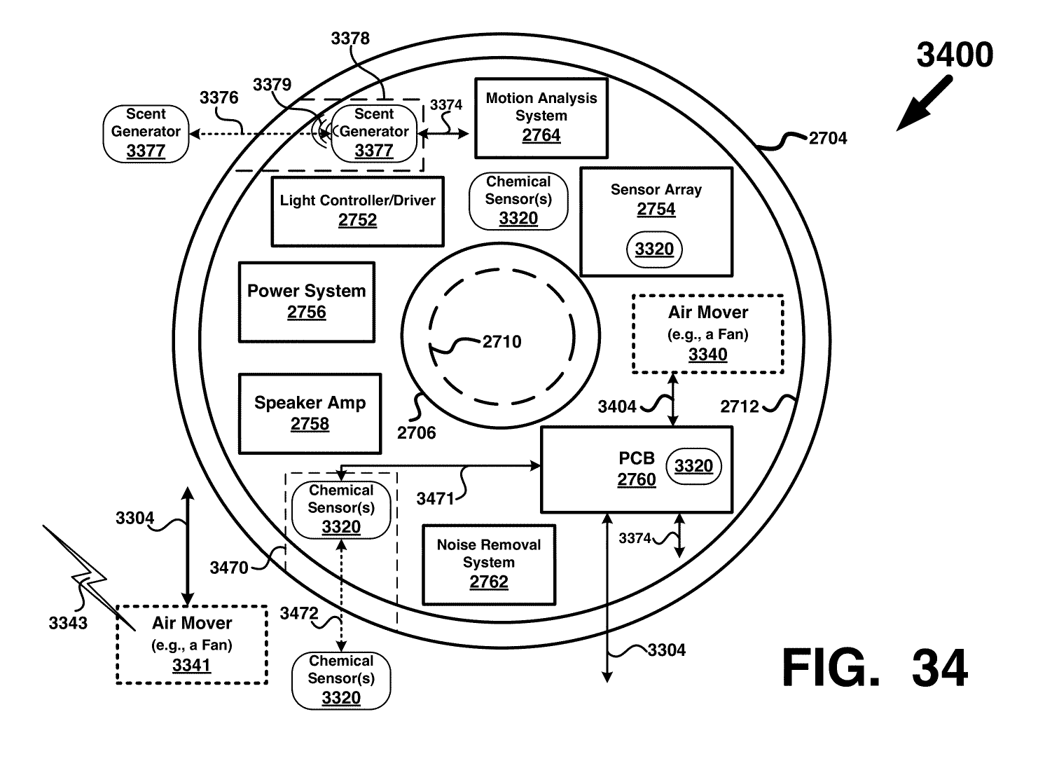

FIG. 32 depicts an exemplary system 3200 for controlling a combination speaker and light source device according to a physiological state and/or chemicals sensed by a chemical sensor (also referred to as an environmental sensor). Here, system 3200 may include the wearable device 3002, the mobile device 3004 and one or more speaker-light devices 3206 and 3208 as was described above in reference to FIG. 30. One or more of the devices may be in wired and/or wireless communication with one another as denoted by communications link 3221 (e.g., Bluetooth®, Bluetooth Low Energy, NFC, ultra wideband, Software Defined Radio, WiFi, WiMAX, Cellular, IEEE 802.x, Ethernet, USB, etc.). Like-numbered and named elements may describe the same or substantially similar elements as those shown in other descriptions (e.g., as in FIG. 30 or other figures). Here, speaker-light devices 3206 and 3208 may include one or more chemical sensors 3206h and 3208h. Optionally, the speaker-light devices 3206 and 3208 may also include an air mover 3206i and 3208i operative to couple an air flow 3211 with their associated chemical sensors (3206h, 3208h). Optionally, the speaker-light devices 3206 and 3208 may also include a scent generator (not shown) as will be described below. Air flow 3211 may include chemicals, gases, particulate matter, liquid droplets, or other airborne compounds that may be present in an ambient atmosphere (3201, 3203) where the speaker-light devices 3206 and 3208 are positioned. Although FIG. 32 depicts two speaker-light devices 3206 and 3208, there may be more or fewer speaker-light devices than depicted in the example of FIG. 32. In some examples, the air mover is not included in the speaker-light devices 3206 and 3208 and may be positioned externally to housing 2402, such as a ceiling fan or other device operative to create an air flow.

Chemicals that may be sensed by one or more chemical sensors in a speaker-light device (e.g., by chemical sensors 3206h and/or 3208h) may include but are not limited to carbon monoxide (CO), carbon dioxide (CO₂), oxides of nitrogen (e.g., nitrogen oxide—NO₂, NOx), sulfur dioxide (SO₂), sulfates (SO₄), volatile organic compounds (VOC), ozone (e.g., ground level ozone—O₃), lead (Pb), mercury (Hg), hydrogen fluoride (HF), hydrogen sulfide (H₂S), solid or liquid matter suspended in air (e.g., sub-millimeter matter and/or liquid, aerosols), air pollution (e.g., man-made or naturally occurring), asbestos, chlorofluorocarbons (CFCs), chlorine (CL, CL₂) gas, hydrochloric acid/hydrogen chloride (HCL), hydrochlorofluorocarbons (HCFCs), toxic air pollutants (e.g., pesticides, power plants, industrial chemicals, etc.), methane (CH₄) (e.g., from cattle, livestock, etc.), radon (Rn), second hand smoke from tobacco, off gassing from plastics and other materials, and greenhouse gasses, just to name a few. The one or more chemical sensors included in a speaker-light device may be selected to sense one or more types of atmosphere born compounds, such as gasses, particles, aerosols, for example. The one or more chemical sensors may be selected to sense atmospheric compounds that are of the most concern of a user and/or are most likely to be present at a location (e.g., a house, apartment, workplace) the speaker-light device is installed at. For example, a user who lives close to a cattle ranch may select for installation in his/her speaker-light device a chemical sensor operative to sense methane (CH₄) generated by manure. As another example, a user living in vicinity of a coal fired electrical power generation plant may select a chemical sensor operative to sense carbon dioxide (CO₂) and/or sulfur dioxide (SO₂). One or more of the chemical sensors may be operative to sense chemicals and/or compounds (e.g., VOC) in the atmosphere that may affect sleep in a user (e.g., REM sleep and Non-REM sleep).

In some examples, the chemical sensor(s) may be designed to be removeably interchangeable in the speaker-light device, such that the chemicals to be sensed may be captured by specific suites of chemical sensors that may be inserted into and removed from the speaker-light device. As one example, a speaker-light device may include locations for one or more chemical sensors that may be inserted and removed from the speaker-light device as the sensing needs of the user change. For example, the speaker-light device may include slots, ports, openings, docks, etc. for a plurality of chemical sensors, and a user may select two chemical sensors, one for sensing greenhouse gases carbon dioxide (CO₂) and sulfur dioxide (SO₂) and another for sensing radon (Rn) gas. Later, the user becomes concerned about ozone (O₃) and inserts an ozone chemical sensor into one of the available slots in the speaker-light device. Other examples of modules and/or suites of chemical sensors that may be installed and optionally later removed from the speaker-light device, include chemical sensors operative to detect smoke and/or other atmospheric particulates associated with fire, and chemical sensors operative to detect carbon monoxide (CO) which may be generated by a furnace, water heater, lawn tool, or automobile. In yet other examples, some or all of the chemical sensor(s) may be non-removable from the speaker-light device.

Attention is now directed to FIG. 33 where an exemplary architecture for a combination speaker and light source device 3300 that includes one or more chemical sensors 3320 is depicted. Here, combination speaker and light source device (i.e., speaker-light device) 3300 includes bus 3302, and may include some of the elements described above in reference to FIG. 26, such as, noise removal module 2604, speaker 2606, memory 2608, logic 2610, light control module 2614, light source 2616, communication facility 2618, motion analysis module 2620, and power module 2622. Like-numbered and named elements may describe the same or substantially similar elements as those shown in other descriptions. In some examples, a sensor array 3312 may include one or more of a motion sensor (e.g., accelerometer, gyroscopic sensors, optical motion sensors (e.g., laser or LED motion detectors, such as used in optical mice), magnet-based motion sensors (e.g., detecting magnetic fields, or changes thereof, to detect motion), electromagnetic-based sensors, MEMS, and the like), a chemical sensor (e.g., carbon dioxide (CO2), oxygen (O2), carbon monoxide (CO), airborne chemicals, toxins, and the like), a temperature sensor (e.g., thermometer, temperature gauge, IR thermometer, resistance thermometer, heat flux sensor, and the like), humidity sensor, passive IR sensor, ultrasonic sensor, proximity sensor, pressure sensor, light sensors and acoustic sensors, as described herein, and the like. Sensor array 3312 may include at least one chemical sensor 3320 along with its other sensors, the chemical sensor(s) 3320 may be external to the sensor array 3312 or both. The combination speaker and light source device 3300 may include an internal air mover 3340 coupled with bus 3302. An external air move 3341 may be coupled 3304 with bus 3302 and controlled by logic and/or software in the combination speaker and light source device 3300. Signals from chemical sensor(s) 3320 may be processed by logic 2610 and/or software executing on a compute engine such as a processor (e.g., μP 3350) or the like included with the logic 2610 or disposed elsewhere in the combination speaker and light source device 3300. Although the example of FIG. 33 depicts the elements of combination speaker and light source device 3300 primarily coupled with bus 3302, other configurations may be used and some of the elements depicted may be coupled with one another, such as the light control module 2614 coupled 3306 with the light source 2616, for example. External air move 3341 may be wirelessly coupled 3343 with communications facility 2618 and may have its operation wirelessly controlled via a wireless link with device 3300.

The combination speaker and light source device 3300 may include a scent generator 3377 operative to generate (e.g., disperse as an aerosol, gas, liquid, mist, droplets, or the like) one or more chemicals 3379 that affect a user, such as reducing stress, relaxing the user, increasing focus, concentration, attention, helping the user to fall asleep, or helping the user to awaken from sleep, for example. The aforementioned air mover (internal and/or external) may be used in conjunction with scent generator 3377 to disperse/circulate the one or more chemicals 3379. As will be described below, device 3300 may be in communication with an external scent generator that may be used for the same purposes as scent generator 3377.

Moving now to FIG. 34 where a top-view of an exemplary combination speaker and light source device 3400 that includes one or more chemical sensors 3320 is depicted. Here, speaker-light device 3400 includes housing 2704, speaker 2706, platform 2710 being hidden by speaker 2706, and electronics 2712, including light controller/driver 2752, sensor array 2754, power system 2756, speaker amplifier 2758, printed circuit board (PCB) 2760, noise removal system 2762, and motion analysis system 2764. Like-numbered and named elements may describe the same or substantially similar elements as those shown in other descriptions and may include more or fewer elements than shown in other descriptions (e.g., see FIG. 27C). One or more chemical sensors 3320 may be disposed in device 3400, and optionally, one or more air movers 3340 may be disposed in device 3400. PCB 2760 may include one or more of the other elements depicted in device 3400, such as motion analysis system 2764, light controller/driver 2752, or sensor array 2754, for example. PCB 2760 may include one or more environmental sensors, such as chemical sensors 3320. In some examples, one or more of the chemical sensors 3320 may be included in the sensor array 2754. PCB or other circuitry in device 3400 may be coupled (3404, 3304) with internal and/or external air movers (3340, 3341) to control operation of and/or determine status of the air movers.

In some examples, one or more of the chemical sensor(s) 3320 may be removable from the device 3400. As one example, a chemical sensor operative for sensing carbon monoxide (CO) gas may be removed and replaced with an upgraded version of the CO sensor or replaced with a different type of sensor, such as one operative to sense oxides of nitrogen (NOx) or carbon dioxide (CO₂). Removable sensors 3320 may also allow for servicing and/or replacing defective sensors or expired sensors (e.g., a smoke and/or fire sensor may need replacement approximately every 5 years). In FIG. 34, device 3400 may include a slot, docking port, or the like, denoted as 3470, and sensor 3320 may be inserted or removed in the slot as depicted by dashed line 3472. When inserted in 3470, sensor 3320 may electrically connect with circuitry in device 3400 via connection 3471 which may be coupled with circuitry on PCB 2760, for example. Removable/replaceable sensors 3320 may be in a form of a cartridge or other structure that may include electrical nodes that connect with a connection 3471 when the sensor 3320 is inserted into slot 3470. A connector including but not limited to Universal Serial Bus (USB), Lightning, RS-232, XLR, RCA, TRS, TRRS, DIN, 3.5 mm plug, or other may be used to establish an electrical connection between the removable/replaceable sensors 3320 and systems (e.g., PCB 2760) of the device 3400.

Similarly, device 3400 may include one or more scent generators 3377 operative to emit or generate a scent 3379, and the scent generator 3377 may be removable from a slot, docking port, or the like, denoted as 3378. Scent generator 3377 may be inserted or removed in the slot 3378 as depicted by dashed line 3376. Removable/replaceable scent generators 3377 may be in a form of a cartridge or other structure that may include electrical nodes that connect with a connection 3374 when the scent generator 3377 is inserted 3376 into slot 3378. A connector including but not limited to Universal Serial Bus (USB), Lightning, RS-232, XLR, RCA, TRS, TRRS, DIN, 3.5 mm plug, or other may be used to establish an electrical connection between the removable/replaceable scent generators 3377 and systems (e.g., PCB 2760) of the device 3400.

The air mover described above may comprise a fan or other device operative to generate an air flow. The chemical sensor may be positioned within housing (e.g., 2402, 2704) at a location operative to couple air flow 3211 with the chemical sensor (e.g., sensors 3206h, 3208h, 3320) positioned to be in fluid communication with air flow 3211 from ambient 3201, 3203). The air mover (e.g., 3206i, 3208i, 3340) may be operated continuously or periodically and operation may be controlled by logic 2610 (e.g., a processor, a controller, FPGA, μC, μP, etc.) or other circuitry and/or software in the speaker-light device. In other examples, the air mover may be positioned external to the speaker-light device and may be controlled by the speaker-light device or by some other device (e.g., a switch for a fan or ceiling fan). Examples of external air movers include a fan, a HVAC system, and a ceiling fan. Housing 2402 may include slots, opening, vents, etc. that allow for air flow over the chemical sensors either by an internal air mover, an external air mover or both. An external air mover may be controlled (e.g., turned on, turned off, have its speed/flow rate varied) by the speaker-light device. For example, the speaker-light device may be mounted to or in proximity of a ceiling fan and a signal(s) from the speaker-light device may be used to activate/deactivate the ceiling fan and may also control fan speed and/or direction of rotation of the ceiling fan (e.g., to move air up or down).

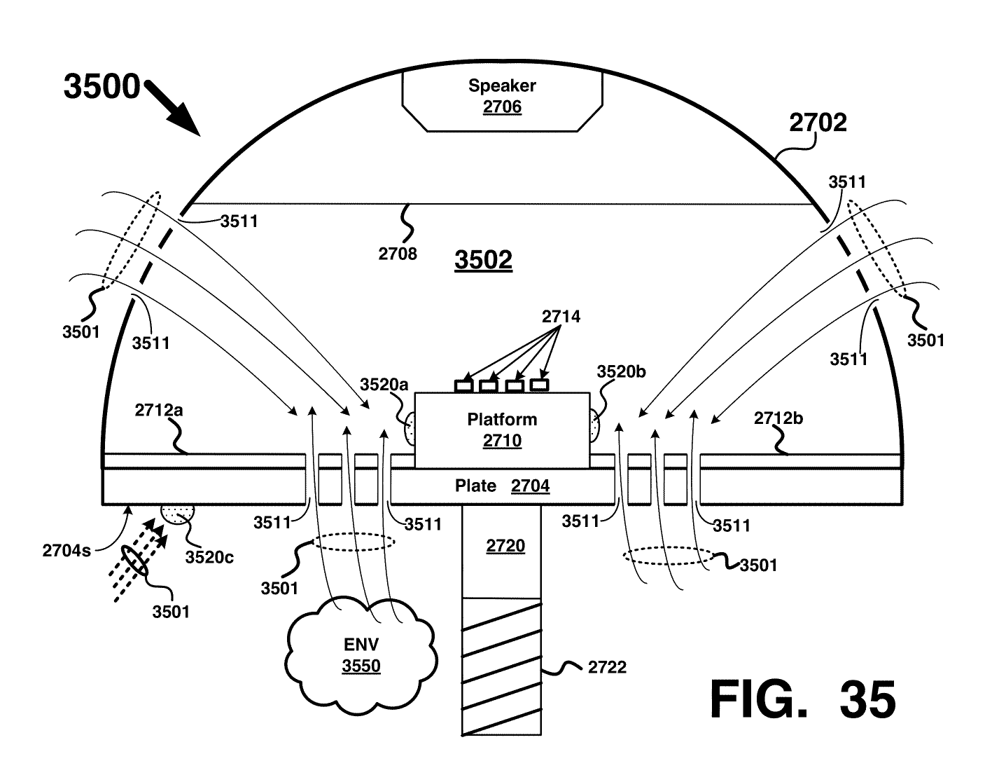

In FIG. 35, a cross-sectional view of an exemplary combination speaker and light source device 3500 includes one or more chemical sensors (3520a, 3520b) that may be positioned on or in the housing 2702 (e.g., disposed in an interior portion 3502 of the device 3500). Housing 2702 may include one or more apertures 3511 (e.g., vents, slots, through holes, grating, portals, etc.) formed in one or more portions of the housing 2702 or other structures (e.g., 2712a, 2712b, 2704) coupled with the housing 2702. Ambient atmosphere 3501 may enter and/or pass through the interior portion 3502 through the one or more apertures 3501 and couple 3211 with (e.g., come into contact with) the one or more chemical sensors (3520a, 3520b). The one or more chemical sensors (3520a, 3520b) may be positioned anywhere within or on device 3500 and the configuration depicted in FIG. 35 is just one non-limiting example and other configurations may be implemented. As one example, an exterior portion of housing 2702 (e.g., a surface 2704s of plate 2704) may include one or more chemical sensors 3520c connected with the exterior portion. The exterior portion may be selected to ensure an effective coupling 3211 between the ambient atmosphere (e.g., the air) and the chemical sensor (e.g., 3520c). In some examples, air flow in an environment (ENV) 3550 the combination speaker and light source device 3500 is disposed in is sufficient to provide an adequate flow of the ambient atmosphere 3501 over the one or more chemical sensors so that the ambient 3501 couples 3211 with (e.g., comes into contact with/passes over or flows over) the chemical sensor(s). Locations for the one or more apertures 3511 on housing 2702 may be application dependent and the examples depicted in FIG. 35 for device 3500 are non-limiting examples and other locations and/or configurations for the apertures 3511 may be implemented. Ambient atmosphere 3501 may flow into and/or flow out of the interior portion 3502 of device 3500 via the apertures 3511, and those flows may be due to natural air currents (e.g., currents generated by conditions in the ambient) or conditions in ENV 3550, may be due to a forced air flow from one or more air movers as will be described below, or both.

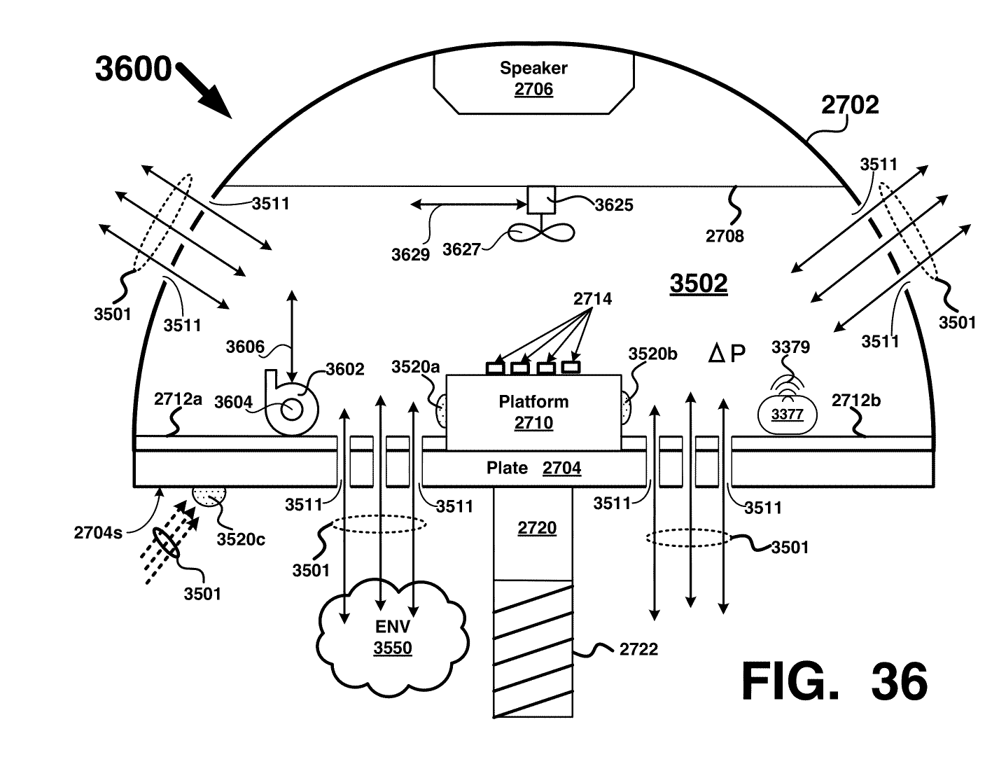

Turning now to FIG. 36 where a cross-sectional view of an exemplary combination speaker and light source device 3600 depicts one or more chemical sensors and one or more air movers included with the device 3600. Here, as one example, an air mover 3602 (e.g., a radial blower fan) may be disposed in the interior portion 3502 and may be coupled 3606 with circuitry operative to activate the blower 3604 to generate flow 3511 into or out of interior portion 3502 (e.g., by generating a differential pressure gradient AP in 3502) such that the ambient atmosphere 3501 couples with the one or more chemical sensors as described above. As another example, an air mover 3625 may be disposed in the interior portion 3502 and may be coupled 3629 with circuitry operative to activate the fan blades 3627 to generate flow 3511 into or out of interior portion 3502. Circuitry for controlling the air movers may be disposed on PCB 2760 of FIG. 34 or in logic 2610 of FIG. 33, for example. The types and positions of the air movers depicted in FIG. 36 are non-limiting examples and other types and positions (e.g., exterior mounted air movers) may be implemented. Flow 3551 may be used for alternative and/or additional purposes, including but not limited to removing waste heat generated by components of the combination speaker and light source device 3600 (e.g., for thermal cooling), purging or cleansing the chemical sensors of contamination, dust, particulates, etc., just to name a few, for example. The air movers may be activated continuously, intermittently, on demand, or on an as needed basis, for example.

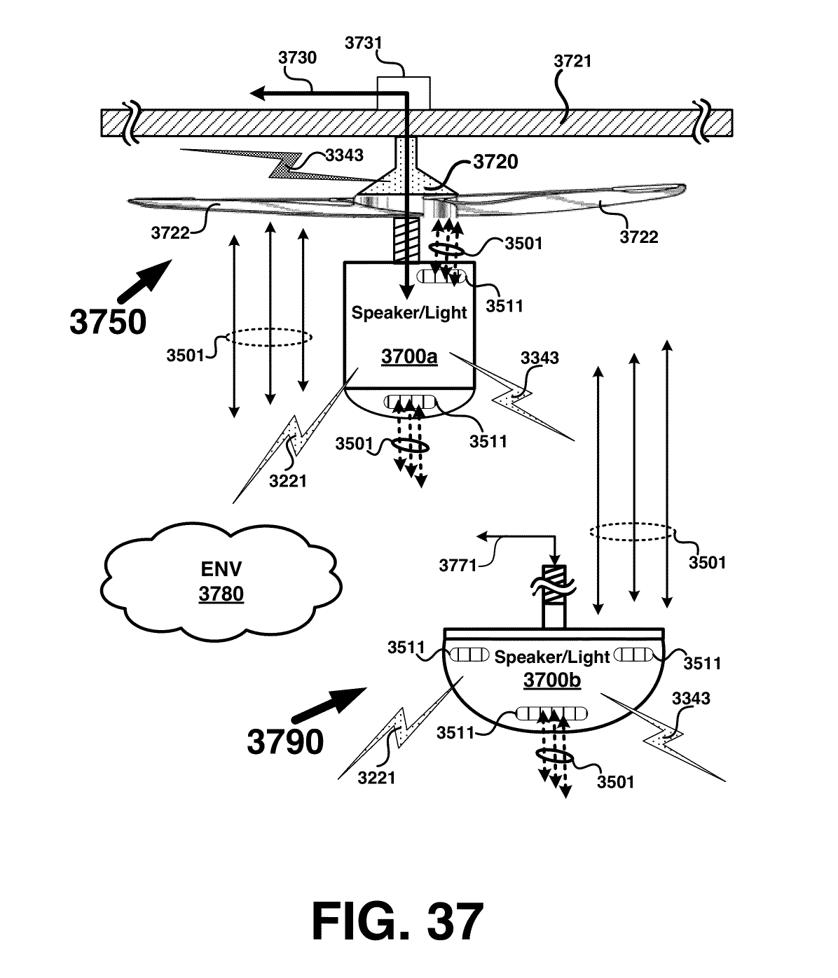

Referring now to FIG. 37 where a cross-sectional view an example 3750 of a combination speaker and light source device including a chemical sensor 3700a coupled with an external air mover 3720 and an example 3790 of a combination speaker and light source device including a chemical sensor 3700b positioned in proximity of the external air mover 3720 are depicted. Here, chemical sensors (3700a, 3700b) may sense environment 3780 for chemicals or other compounds as described above. In example 3750 the air mover 3720 may be a ceiling fan mounted to a structure 3721. An electrical connection 3730 may provide power and/or control signals for air mover 3720 and may also provide power and/or control signals for device 3700a. The electrical connection 3730 may be routed through an electrical junction box 3731 connected with structure 3721, for example. Wireless links 3321 and/or 3343 as described above may also be used to communicate commands, control, data, and other signals between air mover 3720 and/or device 3700a. Here at least a portion of flow 3501 generated by movement of blades 3722 of air mover 3720 may flow over device 3700a and/or flow through apertures 3511 in device 3700a and couple with chemical sensor disposed internally and/or externally in device 3700a.

Alternatively or in addition to device 3700a, device 3700b may be disposed in proximity of an air flow 3501 of an external air mover, such as air mover 3720. For example, device 3700b may be mounted on a ceiling or a wall that is in close enough proximity to air mover 3720 to receive at least a portion of flow 3501. Here flow 3501 generated by movement of blades 3722 of air mover 3720 may flow over device 3700b and/or flow through apertures 3511 in device 3700b and couple with chemical sensor disposed internally and/or externally in device 3700b. Commands, control, data, and other signals may be communicated between air mover 3720 and/or device 3700a using one or more of a wired link 3771, wireless link 3321, or wireless link 3343. Examples of commands, data, control and other signals for device (3700a, 3700b) include but are not limited to turning air mover on or off, controlling fan speed, controlling direction of rotation (e.g., CW or CCW to set direction of flow 3501) of blades 3722, controlling fan speed as a function of ambient temperature, controlling fan speed as a function of device (3700a, 3700b) temperature, activating (e.g., turning air mover “ON”) or deactivating (e.g., turning air mover “OFF”) the air mover 3720 for chemical sensing by specific chemical sensor(s) in device (3700a, 3700b), just to name a few.

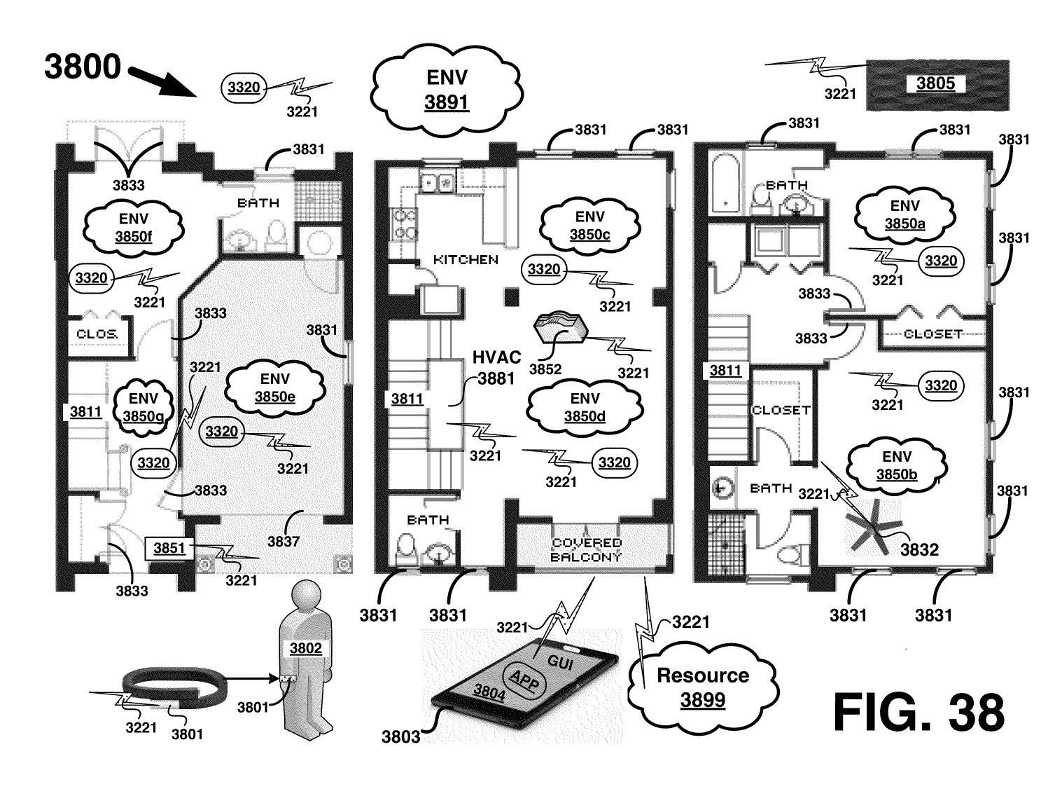

Turning attention now to FIG. 38 where top plan views of a structure 3800 including a plurality of combination speaker and light source devices positioned in a plurality of rooms on different floors of the structure 3800 are depicted. Here, instead of depicted all of the components of each combination speaker and light source device, each device is simply denoted by its respective chemical sensor(s) 3320. In FIG. 38, structure 3800 may be a residential home or other form of structure that is occupied by users and optionally their client devices, such as smartphones, tablets, pads, media devices, wireless media devices, wireless routers, etc. Structure 3800 is a non-limiting example presented for purposes of explanation and other structures having different configurations and different numbers of plurality of combination speaker and light source devices.

From a bottom to a top of the drawing sheet for FIG. 38, structure 3800 includes a ground floor, a middle floor, and a top floor linked by staircase 3811. Each floor includes a plurality of windows 3831 and a plurality of doors 3833. Each floor also includes one or more interior environments denoted as ENV 3850a-3850g that may be monitored by one or more chemical sensors 3320 positioned in proximity of those environments. One or more environments external to structure 3800 (e.g., patio, back yard, porch, front yard, parking lot, driveway, etc.) may be monitored by one or more chemical sensors 3320, such as ENV 3891, for example. Each chemical sensor 3320 may be in wireless communication 3321 with other wireless resources, including but not limited to resource 3899 (e.g., the Cloud, the Internet, web site, web page, NAS, data storage, server, PC, laptop, wireless client device, etc.), monitoring device 3851 (e.g., home automation system, building automation system, HVAC controller, climate control system, alarm system, etc.), HVAC system 3881 (e.g., furnace, AC, heating, cooling, air filtration, humidifier, etc.), and network router (wired and/or wireless) 3852.

Each combination speaker and light source device as represented by its respective chemical sensor 3320 may be aware of locations of other combination speaker and light source devices via information communicated to those devices over the wireless links 3321, information included in data storage in those devices, information communicated to those devices from another device, such as client device 3803, media device 3805, wearable device 3801, and resource 3899, for example. Each combination speaker and light source device as represented by its respective chemical sensor 3320 may be aware of locations of one or more users 3802 and/or devices associated with the one or more users, such as client device 3803 (e.g., a smartphone) and/or wearable device 3801 (e.g., a data capable strap band) donned by a user (e.g., on a wrist or other portion of the user's body). User data including but not limited to sleep activity, sleep behavior, quality of sleep, time of sleep, REM sleep, non-REM sleep, accelerometry (e.g., from motion sensor signals), biometric data, arousal of the sympathetic nervous system (SNS), number of steps (e.g., from walking/running), exercise, calorie intake, calorie expenditure, diet, hydration, almanac data, and other user specific data, may be captured and/or be otherwise accessible by the wireless devices depicted in FIG. 38 and may be used by one or more of the combination speaker and light source devices in conjunction with their respective chemical sensors 3320 to communicate data to a user, generate alarms, generate notifications, provide emergency instructions (e.g., in event of a fire, smoke, toxic gas/chemical levels), take or suggest an action beneficial to the health and/or wellbeing of the user, or make a recommendation to a user, for example.

A map of structure 3800 and locations of combination speaker and light source devices (e.g., 3320) may comprise data included in an application (APP), an application programming interface (API), data structure, algorithm or other form in a device or devices including but not limited to one or more of the combination speaker and light source devices (e.g., 3320), client device 3803, wearable device 3801, media device 3805, and resource 3899, for example. As one example, an APP executing on a processor of client device 3803 may include the map of structure 3800 and may also include locations of the chemical sensors 3320. APP may be embodied in a non-transitory computer readable medium residing in client device 3803 are accessible to client device (e.g., via wireless link 3321). One or more of the combination speaker and light source devices 3320 may also include (e.g., in non-volatile memory) or have access to (e.g., via client device 3803, resource 3899) the map of structure 3800 and may use data in the map to route the user to different rooms, spaces, or other environments internal to and/or external to structure 3800 based on signals sensed from chemical sensors 3320 and/or other systems in the combination speaker and light source devices.

The following are non-limiting examples of how the chemical sensors 3320 in one or more of the combination speaker and light source devices may operate to monitor the ambient (3201, 3501) in their respective environments and take one or more actions, if any, upon sensing chemicals of concern to a user or that may be harmful to the user. As a first example, if user 3802 is sleeping in a bedroom on the upper floor and ENV 3850a of the bedroom includes an above nominal concentration of carbon dioxide (CO₂) as detected by chemical sensor 3320, then the combination speaker and light source device associated with sensor 3320 may notify the user 3802 via sound, audio, light, colors of light, varying intensity and/or color of light, in an electronic message (e.g., email, text message, voice mail, etc.) to open one or more of the windows 3831 in the bedroom to increase air circulation in ENV 3850a. Other chemical sensors 3320 may be queried to determine if the proposed action (e.g., opening windows 3831) may be effective or may make matters worse. For example, chemical sensor 3320 positioned in external environment ENV 3891 may be queried to determine that a source of the CO₂ gas is not coming from a source outside of the structure 3800 such that opening the windows 3831 will make matters worse by potentially further increase concentration of the CO₂ gas in ENV 3850a. In some examples the notification may be designed to awaken the user 3802 so that the suggested action may be taken immediately; whereas, in other examples the notification may occur after the user 3802 has awaken and the user 3802 may take future actions based on the notification, such as opening or more of the windows 3831 prior to going to sleep or taking a nap. An icon (e.g., of a fan) may be presented to the user 3802 on a device, such as client device 3803, and the icon may be used to inform the user that the fan was turned ON to decrease a concentration of some chemical(s), such as the above described CO₂ gas.

As a second example, the user 3802 may be studying in another room in the upper floor where a chemical sensor 3320 in ENV 3850b detects chemicals in the ambient that are typically associated with outgassing (e.g., from newly laid carpet or plastics). Here, accelerometry from motion signals generated by motion sensors in wearable device 3801 may indicate the user 3802 is becoming sluggish and that sensor data may be synthesized along with other data, such as the detection of the outgas chemicals, to determine that a possible cause of the sluggishness may be due a physiological change in the user 3802 from breathing the outgas chemicals. The determining and/or data synthesis may be performed on a processor in one or more of the combination speaker and light source devices, the client device 3803, media device 3805, wearable device 3801, an external resource (e.g., resource 3899), or some combination of the foregoing. An action taken by one or more of the combination speaker and light source devices (e.g., associated with chemical sensor 3320 in ENV 3850b) may comprise sending a text message to user 3802's client device 3803 instructing the user 3802 to open one or more windows 3381, to leave the room for another room, to turn on ceiling fan 3832, to turn on fan 3832 and open windows 3381, for example. In another example, the action taken may comprise one or more of the combination speaker and light source devices notifying the user and/or automatically and without user 3802 intervention, causing the fan 3832 to turn “ON” to circulate air throughout ENV 3850b. In yet other examples, opening and closing of one or more of the windows 3831 may be controlled by a control system 3851 and one or more of the combination speaker and light source devices may communicate 3321a signal to control system 3851 that commands the control system 3851 to open windows 3831 in the room for ENV 3850b. Another signal may command the control system to turn ON/OFF the fan 3832. Opening the windows 3831 and turning ON fan 3832 may be actions taken without intervention on part of user 3802, and may be operative to increase air circulation in ENV 3850b and may allow for removal or reduction of the chemicals from the outgassing. Fan 3832 may comprise fan 3700a or 3700b described above in reference to FIG. 37.

As a third example, chemicals and gasses associated with smoke and/or fire that may be detected by chemical sensors 3320 positioned in ENV 3850c and/or ENV 3850d may trigger an alarm or other warning signal on one or more devices such as the combination speaker and light source devices, media device 3805, client device 3803, wearable device 3801, control system 3851 or others. One or more of the combination speaker and light source devices may also communicate to the user 3802 a safe escape route away from the smoke/fire and/or out of structure 3800. The combination speaker and light source devices may query one another and may operate to generate an escape route based on combination speaker and light source devices that are not detecting the smoke/fire.

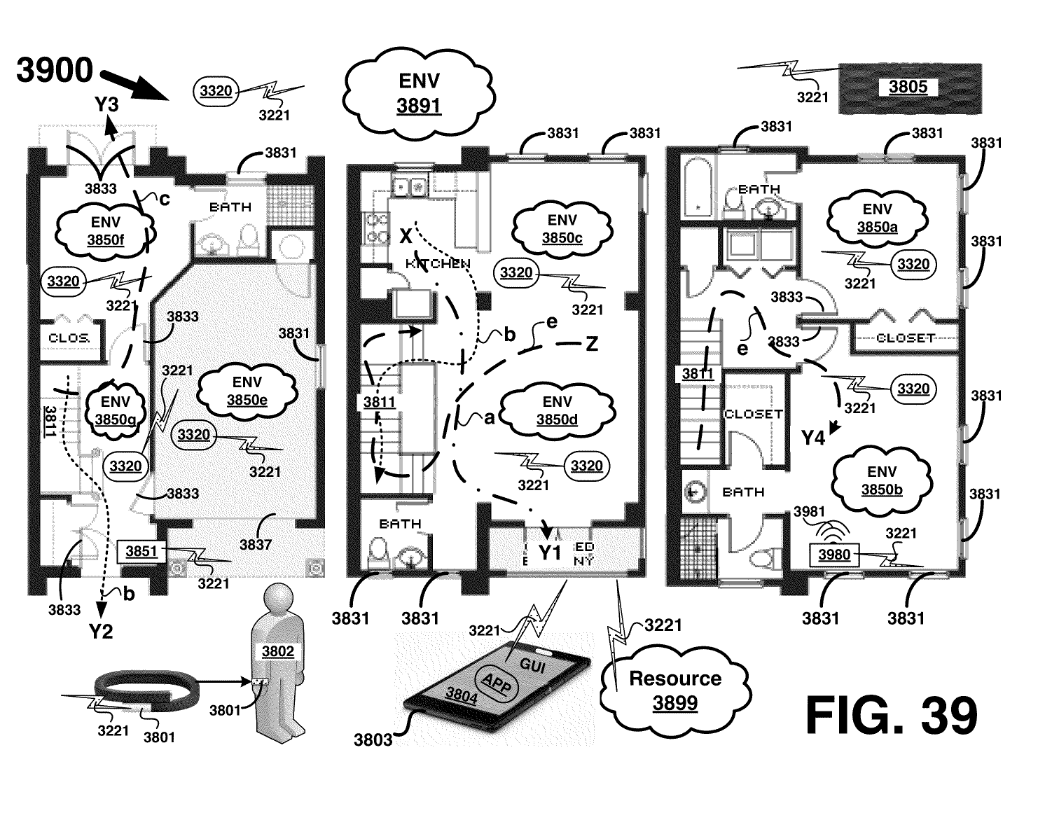

To further illustrate examples of possible routes that may be generated and communicated to the user 3802 and/or a device accessible by the user 3802, attention is now directed to FIG. 39 where examples of routes a-c generated by one or more combination speaker and light source devices are depicted. Here, starting from a point of X potential danger (e.g., fire/smoke in kitchen on middle floor) for the user 3802, assume for purposes of explanation that the user 3802 is at or near point X at a time the fire/smoke breaks out in the kitchen and chemicals from the fire/smoke are detected by chemical sensors 3320 in ENV 3850c and/or 3850d. Here, foreknowledge of a layout of structure 3800 may be used by one or more of the combination speaker and light source devices and/or another device (e.g., client device 3803 via the APP). Some or all of the combination speaker and light source devices may be queried to determine which chemical sensors 3320 in which ENV's are detecting chemicals harmful to user 3802. Locations of chemical sensors 3320 that detect harmful chemicals may be compared with locations of chemical sensors 3320 that are detecting harmful chemicals and a route(s) may be calculated through structure 3800 for the user 3802 to follow away from areas of danger (e.g., X in the kitchen) and to areas of safety (e.g., points Y1, Y2, Y3).

As one example, path a may be calculated for user 3802 to follow from X to a point Y1 on a balcony of the middle floor where rescue may be possible by first responders. As another example, path b may be calculated for user to follow from X to a point Y2 on the first level using stair well 3811 as an escape route to a front door 3831 of structure 3800. As yet another example, path c may be calculated for user to follow from X to a point Y3 on the first level using stair well 3811 as an escape route to a patio door 3831 of structure 3800.

Routing may occur for other than emergency situations, for example, consider route e from a point Z on the middle floor, up stairwell 3811 to a point Y4 on the upper floor. Here, route e may be calculated based on chemical sensor 3320 in ENV 3850d detecting chemicals/gasses associated with tobacco smoke in ENV 3850d. Route e may be selected based on chemical sensor 3320 in ENV 3850b detecting an ambient that is free of the tobacco related chemicals/gasses.

The routes described above may be presented to user 3802 visually on a display of a device, such as a display 3804 of client device 3803, for example. The routes described above may be presented to user 3802 in one or more other forms including but not limited to verbal instructions, sound, light, and vibration. Another device, such as a mobile device may be used to map locations of one or more of the combination speaker and light source devices at the positions they are disposed at in the ENV's that they monitor and serve. For example, client device 3803 may execute the APP and a GUI on display 3804 may guide user 3802 to position the client device 3802 next to or into contact with each combination speaker and light source device on each floor of structure 3800. GPS or other location based systems on client device 3803 or accessible (e.g., via link 3321) may be used to determine locations of each combination speaker and light source device in structure 3800. Client device 3803 may also be moved along a perimeter of major walls or other structures within structure 3800 to map locations of walls, doors, windows, stairwells and other relevant areas of structure 3800.

Detection of other chemicals such as carbon monoxide (CO) may also trigger an emergency alarm and may result in one or more possible safe routes being calculated and presented to the user 3802. For example, detection of carbon monoxide (CO) in ENV 3850e in a garage on the lower floor by a chemical sensor 3320 may result in automatic raising of a garage door to reduce a concentration of the CO and may also result in calculation of safe routes away from CO contaminated areas of structure 3800 to other safe areas as described above. In that some chemicals, such as CO for example, may be denser than the ambient air, a fan such as described above may be activated to draw and/or mix up (e.g., circulate) ambient air in order to determine if that air includes a lower layer of CO or other denser chemical(s) that may not rise up to a level of the chemical sensor 3320. Chemicals other than CO may be present and the above description using CO is a non-limiting example of one type of chemical that may be detected using one or more chemical sensors 3320 in one or more combination speaker and light source devices. Not all of the chemicals that may be detected by the using one or more chemical sensors 3320 need be harmful or toxic to the user 3802. Chemicals that may be detected by the one or more chemical sensors 3320 may include but are not limited to chemicals that may affect physical health, mental health, one or more parameters related to sleep, ability to stay away, awareness, attention span, stress, relaxation, mood, or one or more biometric parameters of the user's 3802 body (e.g., respiration, heart rate, blood pressure, arousal of the SNS, etc.), for example.

Although the foregoing example described a scenario were an emergency condition that may affect the user's 3802 health, safety, or welfare may result in one or more combination speaker and light source devices taking an action(s), such as presenting a escape route from an area of danger to an area of safety. However, a non-emergency situation in the environment of the user 3802 may also result in one or more combination speaker and light source devices analyzing using their various systems, such as chemical sensors and others, the environment and detecting a chemical(s) (e.g., CO₂) know to make the user 3802 drowsy and less productive. Drowsiness may be detected using other sensors in one or more other devices, such as wearable device 3801 (e.g., using accelerometry from motion sensors) and/or media device 3805 (e.g., using proximity sensors and/or passive motion sensors). Historical data (e.g., from an almanac of user data about user 3802) may be used to correlate the drowsiness with the presence of the chemical(s) (e.g., CO₂) and analysis by the one or more combination speaker and light source devices and/or an external device (e.g., a server or Cloud resource) may result in an action to be taken by the one or more combination speaker and light source devices, such as presenting a suggestion to the user 3802 to move from the environment (e.g., ENV 3850a) where the chemical(s) causing the drowsiness are present to another environment (e.g., ENV 3850d) where no such chemical(s) are detected as being present. The information presented may also include a route (e.g., via stairwell 3811) from the unfavorable environment (e.g., ENV 3850a) to the more favorable environment (e.g., ENV 3850d). In other examples, detection of one or more chemicals may result in the one or more combination speaker and light source devices accessing (e.g., using wired and/or wireless communications) one or more other systems such as a fan (e.g., ceiling fan 3832), HVAC system 3881, opening/closing a window 3831, opening/closing a door (e.g., garage door 3837), activating a security system (e.g., an alarm), transmitting a message to a security service and/or first responder (e.g., ADT®, Fire, Police, Paramedics, 911, etc.), transmitting a message to a client device (e.g., device 3803), just to name a few.

One or more combination speaker and light source devices may learn over time, using any number of data inputs and one or more of their respective systems, how various chemicals detected by their chemical sensors 3320 affect a user. Initially, a correlation between a detected chemical and its effect on the user may not be immediately determinable; however, over time the correlation may be determined by analyzing user almanac data (e.g., sleep data) for a pattern or other form of data signature that manifests at or around the time the chemical is detected. For example, if chemicals from tobacco smoke are detected and over time the user's sleep patterns indicate the user is restless during sleep (e.g., from captured motion data), does not sleep for as long (e.g., temporal data, motion data, biometric data), then a correlation between the tobacco smoke and the user data may be correlated to determine that if tobacco smoke is present the user will not sleep well and a suggested course of action may be presented to the user (e.g., via a smartphone, a media device, a speaker in the combination speaker and light source device, a vibration in a wearable device, etc.).

Almanac data about a user may include relevant medical history, medical data, and other health related data that may be used to determine what impact (positive or negative) that a presence of one or more detected chemicals may have on the user. A data store such as the Cloud, NAS, the Internet, memory internal to combination speaker and light source device, and As one example, if the almanac data indicates the user has a history of respiratory illness or asthma, then detection of chemicals known to be detrimental to the user may result in an appropriate action, such as suggesting the user keeps his/her inhaler in close proximity in case an asthma attack may be caused by the detected chemicals, or activating an air filtration system to scrub or remove the harmful chemicals from the environment the user is in. In some examples, a chemical detected by chemical sensor(s) 3320 may be perceived as a foul or irritating odor to a user, such as in the case of industrial pollution from farming or raising livestock/cattle. The almanac data may include information on the user's sensitivity to those odors and an action taken may include the combination speaker and light source device(s) activating an air filtration unit, a fan, or a scent generator (3377, 3980) (e.g., an air freshener) to emit chemicals (3379, 3981) that may counter to odor caused by the chemicals (see scent generator 3990 in ENV 3850b in FIG. 39). In some examples the scent generator 3980 may be external to the combination speaker and light source device(s) as depicted in FIG. 39, may be internal to the combination speaker and light source device(s) (e.g., scent generator 3377 as depicted in FIG. 33), or both. Scent generation may be used to affect a mood of the user and/or induce a desired behavior in the user, such as emitting chemicals 3981 designed to relax the user when sensor data indicates the user is stressed, emitting chemicals 3981 designed to cause the user to sleep, emitting chemicals 3981 designed to awaken the user from sleep, or emitting chemicals 3981 designed to increase concentration, focus, or attention in the user, for example. Scent generation may be used in concert with other stimulus from the combination speaker and light source device(s) and/or other devices in communication with the combination speaker and light source device(s), such as sound, music, light, color of light, color temperature of light (e.g., from about 2700K to about 6000K), vibration, presentation of information on a visual display (e.g., screen 3804 of device 3803), or some combination of the foregoing.

In other examples a chemical that is detected by chemical sensor(s) 3320 may be of known danger to the user (e.g., fire, smoke, industrial chemicals, toxic chemicals) and immediate action to notify the user and/or others may be taken by one or more combination speaker and light source devices and/or other devices in communication with the one or more combination speaker and light source devices. A structure that includes the one or more combination speaker and light source devices and their associated chemical sensors 3320 need not be a building or other types of terrestrial structures, but may also include without limitation, vehicles, open spaces such as parking lots, parks, fields, stadiums, plazas, just to name a few.

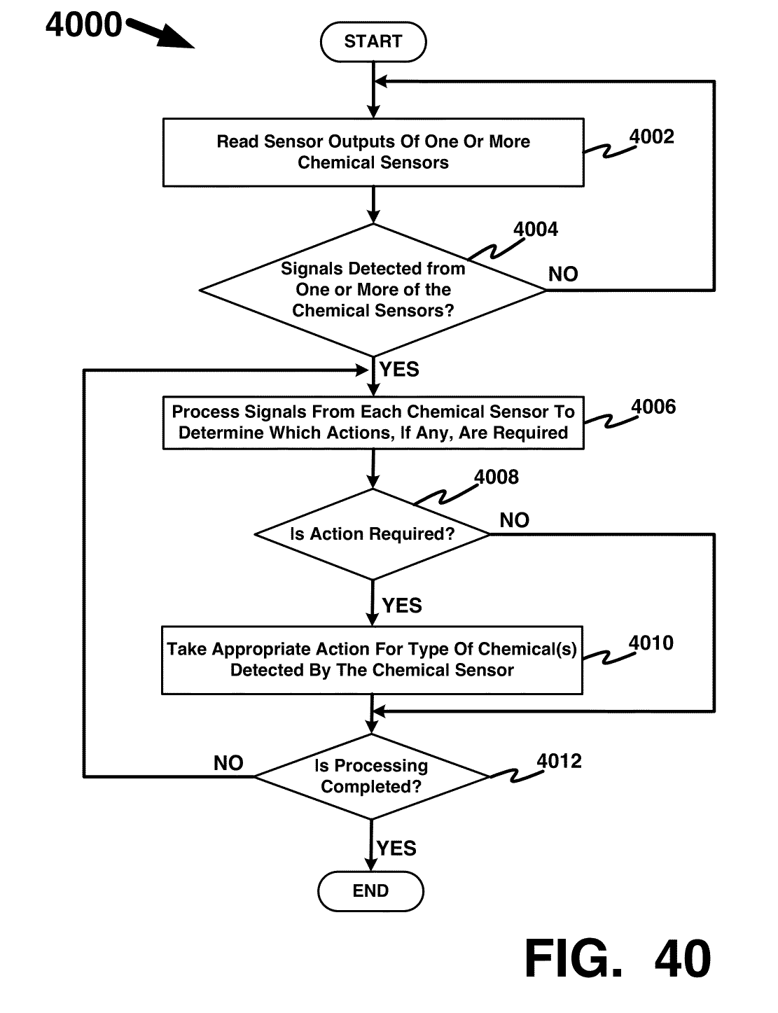

Attention is now directed to FIG. 40 where an exemplary flow 4000 for controlling a combination speaker and light source device according to detection of one or more chemicals by one or more chemical sensors as described above is depicted. At a stage 4002 outputs of one or more chemical sensors (e.g., a signal on 3471 of chemical sensor 3320 in FIG. 34) may be read (e.g., by circuitry and/or processors). Here, reading of output signals from the one or more chemical sensors may comprise reading all of the outputs of all of the chemical sensors at the same time or nearly simultaneously, may comprise reading the outputs of one or more of the chemical sensors in some sequence, for example.

At a stage 4004 a determination may be made as to whether or not signals have been detected on one or more of the chemical sensors (e.g., a signal detected on an output of a chemical sensor(s)). If no signals have been detected, then a NO branch may be taken to another stage of flow 4000, such as the stage 4002 where outputs from the one or more chemical sensors may continue to be read. If signals have been detected on an output(s) of one or more of the chemical sensors, then a YES branch may be taken to a stage 4006.

At the stage 4006, chemical sensors for which an output signal has been detected have their respective signals processed to determine which action(s), if any, are required to be taken by one or more combination speaker and light source devices and/or by other devices in communication with the one or more combination speaker and light source devices. Processing of the signals may occur internally to the one or more combination speaker and light source devices, externally to the one or more combination speaker and light source devices, or both. Processing may comprise hardware, software or both. Processing may occur internally (e.g., logic 2610 and/or μP 3360) and/or externally (e.g., resource 3899) to the combination speaker and light source devices. Data used for the processing and/or the determining whether or not an action is to be taken may include data from internal and/or external sources including but not limited to resource 3899, memory 2608, the Cloud, the Internet, user almanac data, a data store, database, NAS, RAID, a sever, a client device, etc. Algorithms and/or data embodied in a non-transitory computer readable may be applied to the processing and/or the determining and different algorithms and/or data may be applied to the signals from different types of chemical sensors (e.g., CO sensor vs. NO sensor).

At a stage 4008 if action is required, then a YES branch may be taken to a stage 4010. If no action is required, then a NO branch may be taken to another stage in flow 4000, such as a stage 4012. At the stage 4010, an appropriate action for the type(s) of chemicals detected by each chemical sensor may be taken by one or more combination speaker and light source devices and/or by other devices in communication with the one or more combination speaker and light source devices. For example, if a combination speaker and light source device include four (4) chemical sensors and two (2) of those sensors had signals on their outputs that were processed and determined to require action, then at the stage 4010 the appropriate action by the appropriate devices may be taken. In this example, different appropriate actions may be taken for the two sensors and those actions may be taken by the same or different devices. Further to the example, if a first of the two sensors detected chemicals associated with paint fumes, then the appropriate action may comprise communicating a message to a user to leave the environment (e.g., a room) where the fumes are present, may comprise the combination speaker and light source devices opening windows in a room where the fumes are present and/or turning on a fan in that room to increase air circulation. If a second of the two sensors detects high concentrations of CO gas in a room the user is in, the appropriate action may comprise presenting an emergency exit route to the user (e.g., on display 3804 of client device 3803) and visually and/or audibly guiding the user along the route to a point of safety (e.g., outdoors), opening windows in the room, or sounding an alarm using one of its speakers, by vibrating a wearable device (e.g., wear bale device 3801), or using a sound system in a media device (e.g., media device 3805).

At a stage 4012 if processing of signals is completed, then a YES branch may be taken and flow 4000 may terminate or may transition to a stage in another flow as described above. If processing of signals is not completed, then a NO branch may be taken and flow 4000 may transition to another stage, such as the stage 4006 where processing may continue.

Flow 4000 may occur in parallel or in series with other flows describe above and may use data and signals from other systems internal to and/or external to the combination speaker and light source devices. For example, one or more of data, signals, and processing related to sleep of a user as described above in reference to FIGS. 19-20B may be used to decide whether or not chemicals detected from chemical sensors may affect sleep of a user. An air mover as described above may be activated (e.g., turned ON) or deactivated (e.g., turned OFF) during one or more stages of flow 4000, such as the stage 4002 during the reading or during the stage 4006 during the processing, for example. The appropriate action taken at the stage 4010 may comprise activating (e.g., turning ON) or deactivating (e.g., turning OFF) a scent generator 3377, for example, in response to a chemical sensor 3320 detecting chemicals associated with sulfur (S) (e.g., from industrial pollution) in the environment of the user. While the chemical sensor 3320 detects the sulfur (S) the appropriate action may be to activate the scent generator 3377. When the chemical sensor 3320 no longer detects the sulfur (S) or the concentration of the sulfur is below a threshold value, the appropriate action may be to deactivate the scent generator 3377.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed is:

1. A method, comprising:
reading a sensor output of a chemical sensor included in a combination speaker and light source device;
detecting a signal on the sensor output;
processing, after the detecting, the signal to determine which action or actions, if any, are required to be taken combination speaker and light source device or one or more other devices in wireless communication with the combination speaker and light source device; and
taking an appropriate action for the type of chemical detected by the chemical sensor.

2. The method of claim 1, wherein the appropriate action includes activating an air mover to generate an air flow over the chemical sensor.

3. The method of claim 2, wherein the air mover is included in the combination speaker and light source device.

4. The method of claim 1, wherein the appropriate action includes presenting an exit route on a display of a client device in wireless communication with the combination speaker and light source device.

5. The method of claim 1 and further comprising:
a scent generator in communication with the combination speaker and light source device.

6. The method of claim 5, wherein the appropriate action comprises activating the scent generator to generate a scent.

7. The method of claim 5, wherein the scent generated is operative to counter an odor created by the chemical sensed by the chemical sensor.

8. The method of claim 5, wherein the scent generator is included in the combination speaker and light source device.

9. The method of claim 5, wherein the scent generator is external to the combination speaker and light source device and the communication comprises a wireless communication link.

10. The method of claim 1, wherein the appropriate action further comprises:
accessing almanac data for a user;
processing data relevant to sleep patterns of the user from the almanac data;
determining, based on the processing, what effect, if any, the chemical detected by the chemical sensor has on sleep of the user; and
presenting to the user any effects the chemical detected by the chemical sensor have on the user's sleep.

11. The method of claim 1, wherein the appropriate action includes activating an air mover to generate an air flow in an environment the chemical sensor is disposed in, wherein the air mover is external to the combination speaker and light source device.

12. The method of claim 1, wherein the appropriate action further comprises:
accessing almanac data for a user;
processing data relevant to sleep patterns of the user from the almanac data;
determining, based on the processing, what effect, if any, the chemical detected by the chemical sensor has on sleep of the user; and
taking the appropriate action comprises generating a stimulus operative to cause the user to sleep or to cause the user to awaken from sleep.

13. The method of claim 12, wherein the stimulus comprises generating sound.

14. The method of claim 12, wherein the sound is generated by a device external to the combination speaker and light source device and in wireless communication with the combination speaker and light source device.

15. The method of claim 12, wherein the stimulus comprises generating light from a light source included in the combination speaker and light source device.

16. The method of claim 15 and further comprising:
varying one or more of an intensity of the light, a color of the light, or a color temperature of the light.

17. The method of claim 1, wherein the appropriate action further comprises:
accessing almanac data for a user;
processing data relevant to sleep patterns of the user from the almanac data;
determining, based on the processing, what effect, if any, the chemical detected by the chemical sensor has on sleep of the user; and
taking the appropriate action comprises activating a scent generator operative to generate a scent, the scent operative to cause the user to sleep or operative to cause the user to awaken from sleep.

18. The method of claim 1, wherein the appropriate action further comprises:
causing a window in a structure the combination speaker and light source is disposed in to open or to close.

19. The method of claim 1, wherein the appropriate action further comprises:
activating a heating, ventilation, and air conditioning (HVAC) system in a structure the combination speaker and light source is disposed in.

20. The method of claim 1, wherein the appropriate action further comprises:
activating a security system in a structure the combination speaker and light source is disposed in.
